(19) United States
(12) Patent Application Publication
(10) Pub. No.: US 2018/0369296 A1
(43) Pub. Date: Dec. 27, 2018

(54) Products and Methods for Therapeutic Administration of Microorganisms to Non-Human Animals

(71) Applicant: Animal Microbiome Analytics, Inc., Oakland, CA (US)

(72) Inventors: Holly H. GANZ, Oakland, CA (US); Kari R. GOODMAN, Oakland, CA (US); Alexandra MARTIN, Berkeley, CA (US)

(21) Appl. No.: 15/990,256

(22) Filed: May 25, 2018

Related U.S. Application Data
(60) Provisional application No. 62/511,860, filed on May 26, 2017.

(51) Int. Cl. A61K 35/742; A61K 35/747; A61K 9/20; A61K 9/00; A61P 1/00

(52) U.S. Cl. CPC A61K 35/742; A61K 35/747; A61K 2035/11; A61K 9/0053; A61P 1/00; A61K 9/2004

(57) ABSTRACT

Embodiments of the invention encompass identification and characterization of microbiota having potential or demonstrated therapeutic effects for certain conditions in certain non-human animals. In some embodiments, the identification and characterization of microbiota may include identifying and characterizing the microbiota of potential donors, characterizing the potential donors, and identifying potentially therapeutic microbiota based on analysis of such data from multiple individuals. In some embodiments, the identification and characterization of microbiota may include testing, that is, administering microbiota of potential donors to animals suffering from a disease or condition, documenting any therapeutic response, and optionally identifying and characterizing the microbiota of recipients before and after treatment, and optionally identifying demonstrated therapeutic microbiota. Embodiments of the invention encompass compositions comprising the microbiotia, including solid oral compositions, methods of making these compositions, and methods of treatment of non-human animals suffering from various diseases and conditions with these compositions.

### CROSS-REFERENCE TO RELATED APPLICATION

This application claims priority to and the benefit of U.S. provisional patent application No. 62/511,860, filed on May 26, 2017, which is incorporated by reference herein in its entirety, and expressly including any drawings, for all purposes.

### BACKGROUND

### Field of the Invention

This invention is directed to therapeutic administration of microorganisms to non-human animals, including compositions of microorganisms, such as microbiota, for therapeutic treatment of a disease or condition and products and methods for administration for such therapeutic treatment.

### Description of the State of the Art

The digestive tract of many animals, including humans and other mammals, includes microorganisms such as bacteria that may assist in digestion and support the general health of the animal. Variations in the community of microorganisms—the microbiota—in the digestive tract, including variations in the types and abundance of types of microorganisms, can affect health and wellness. For example, antibiotics may kill off many species of bacteria in the digestive tract, allowing one or more resistant species to dominate the microbiota. In some cases, treatment with antibiotics leads to an explosive growth of Clostridium difficile (C. difficile or C. diff.), which can cause diarrhea, cramping, dehydration, bleeding and even kidney failure. Because these organisms are resistant to antibiotics, other methods of treatment are needed to reduce their growth and re-establish a normal microbiota.

Recently, fecal transplants have been used in humans to treat C. diff infections. The concept is to repopulate the digestive tract of a person suffering from an overpopulation of C. diff with all the microorganisms of the digestive tract of a healthy person. The treatment often involves preparing a saline-based suspension or slurry of a donor's fecal matter and administering the slurry/suspension via an enema or a colonscope/endoscope. The “dose” is often on the order of 45 g to 75 g of human fecal matter. Such methods of treatment are problematic because sourcing, storage, transport, and delivery of the transplant material is logistically difficult, and the experience is invasive and often unpleasant (e.g. because of the odor) for both medical professionals and patients.

Other delivery means, such as delivering a fecal matter solution through a nasogastric tube and/or formulation of the fecal matter into a suppository or gel for rectal administration, have been suggested. Oral compositions such as capsules, tablets, gels, suspensions, gel-tabs and others have been suggested for frozen, freeze-dried, or lyophilized fecal matter compositions. In some instances, capsules filled with an aqueous suspension, gel, semi-solid and/or solid have been suggested. Clinical administration of at least one of these capsule formulations has been reported (see WO 2016/178775 A1).

Relatively little is known about what is in the reported or potential fecal transplants. There has been identification of the families of microbiota present and some characterization of their taxonomic diversity. There has been some evaluation by anaerobic culturing of previously frozen fecal matter samples to assess viability of their microbiota. However, fecal matter for potential transplant is generally identified by health screening of the donor via questionnaire and blood/plasma sampling, and potentially by testing of the fecal matter for known pathogens. There is very limited knowledge of the characteristics of the microbiota transferred or how those characteristics correlate with therapeutic efficacy.

Development and testing of treatments with bacteria that are normally present in the gastro-intestinal system is challenging because many of the bacteria are not known or available as cultures. Probiotics, which may be administered orally as tablets, capsules, etc., and/or which may be added to food, are examples of some of the microorganisms that can be cultured and processed. Probiotics are administered not only to treat gastro-intestinal upset but also to promote and/or maintain digestive health. However, many of the beneficial bacteria present in fecal matter, for example, Oscillospira, have not yet been cultured.

Non-human animals, especially domestic animals, are known to suffer from many diseases and conditions found in humans, including digestive disorders. Although there have been suggestions that methods of treatment of humans with fecal transplantation are broadly applicable to all animals, non-human animals may have different microbiota and/or a different response to treatment of a disease or condition as compared to humans. Methods of administering treatment used in humans may not be possible or efficient with non-human animals. In addition, there are limited data on the potential therapeutic use of particular compositions including fecal matter, or particular microbiota, in non-human animals.

### SUMMARY OF THE INVENTION

Embodiments of the invention encompass identification and characterization of microbiota having potential or demonstrated therapeutic effects for certain conditions in certain non-human animals. In some embodiments, the identification and characterization of microbiota may include identifying and characterizing the microbiota of potential donors, characterizing the potential donors, and identifying potentially therapeutic microbiota based on analysis of such data from multiple individuals. In some embodiments, the identification and characterization of microbiota may include testing, that is, administering microbiota of potential donors to animals suffering from a disease or condition, documenting any therapeutic response, and optionally identifying and characterizing the microbiota of recipients before and after treatment, and optionally identifying demonstrated therapeutic microbiota.

In some embodiments, therapeutic microbiota are identified based upon the identification and characterization of microbiota of potential donors, the characteristics of the potential donors, and the documented therapeutic responses of recipients, including patients, to administration of a microbiota.

Embodiments of the invention encompass identification and characterization of microbiota having therapeutic effects for certain diseases or conditions in certain species, groups or taxa of non-human animals. Conditions include, but are not limited to including, digestive, inflammatory, and other conditions, including both acute and chronic conditions. Non-limiting examples include colitis, constipation, acute or chronic diarrhea, gastritis, gastroenteritis, hemorrhagic gastroenteritis, inflammatory bowel disease, irritable bowel disease, irritable or inflammatory bowel syndrome, pancreatitis, small intestinal malabsorption, vomiting and regurgitation, atopic dermatitis, obesity, and diabetes. Conditions may be caused by a variety of factors, including parasites, such as Tritrichomonas foetus, infectious bacteria, such as Campylobacter and Clostridium perfringens, and viruses, such as Parvovirus. Species, groups or taxa of non-human animals include, but are not limited to including, pets such as cats of various breeds, dogs of various breeds, rabbits, and ferrets, and livestock animals such as pigs, cow, horses, sheep, and goats. Further, these therapies may be of benefit to captive wildlife, such as, but not limited to, cheetahs.

Embodiments of the invention encompass compositions, methods of producing the compositions and administration of the compositions where the compositions include, but are not limited to including, microorganisms of at least one of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, Megamonas, at least one member of the family Ruminococcaceae, Clostridium, Prevotella, at least another member of the family Clostridiaceae, at least another member of the family Lachnospiraceae that is not Blautia and that is not Dorea, and Faecalibacterium.

Embodiments of the invention encompass products for administration of such microorganisms, including compositions for oral administration. Embodiments of the invention encompass treatment regimes, including dosing and time course of treatment. Embodiments of the invention encompass compositions, methods of producing the compositions and administration of the compositions where the compositions include the following taxa: Blautia, a member of Clostridiales, a member of Ruminococcaceae, Eubacterium, Faecalibacterium, Oscillospira, Phascolarctobacterium, a member of Mogibacteriaceae, a member of Succinivibrionaceae, Coprococcus, Roseburia, and Catenibacterium. In some embodiments, for the compositions, the above-listed twelve taxa are the twelve most abundant.

Embodiments of the invention encompass compositions, methods of producing the compositions and administration of the compositions where the compositions are intended for administration to feline patients (or subjects), and the compositions include, but are not limited to including, microorganisms of at least one of the following taxa: Dorea, Ruminoccoccus, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella, Prevotella, Eubacterium, Oscillospira, at least another member of the family Clostridiaceae, at least another member of the family Lachnospiraceae that is not Blautia and that is not Dorea, Ruminococcus, Coprococcus, Parabacteroides, Fusobacterium, Faecalibacterium, Megamonas.

Embodiments of the invention encompass compositions, methods of producing the compositions and administration of the compositions where the compositions are intended for administration to feline patients (or subjects), and the compositions include, but are not limited to including, microorganisms of at least one of the following taxa: Dorea, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella.

Embodiments of the invention encompass compositions, methods of producing the compositions and administration of the compositions where the compositions are intended for administration to feline patients (or subjects), and the compositions include, but are not limited to including, microorganisms of at least one of the following taxa: at least one member of the order Clostridiales, at least one member of the family Ruminococcaceae, Clostridium, at least one member of the family Clostridiaceae, Sutterella.

Embodiments of the invention encompass compositions, methods of producing the compositions and administration of the compositions where the compositions are intended for administration to feline patients (or subjects), and the compositions include, but are not limited to including, microorganisms of at least one of the following taxa: at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, and Megamonas.

Embodiments of the invention encompass compositions, methods of producing the compositions and administration of the compositions where the compositions are intended for administration to feline patients (or subjects), and the compositions include, but are not limited to including, microorganisms, and the administration results in a significant change in at least one of the following microorganisms of the microbiota of the patient: Blautia, Oscillospira, Ruminococcus, Lachnospiraceae g1, Clostridiales f1.

Embodiments of the invention encompass compositions, methods of producing the compositions and administration of the compositions where the compositions are intended for administration to canine patients (or subjects), and the compositions include, but are not limited to including, microorganisms of at least one of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, at least one member of the family Erysipelotrichaceae, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium. Embodiments of the invention encompass compositions, methods of producing the compositions and administration of the compositions where the compositions are intended for administration to canine patients (or subjects), and the compositions include, but are not limited to including, microorganisms of at least one of the following taxa: Ruminoccoccus, at least one member of the family Clostridiaceae, at least one member of the family Lachnospiraceae that is not Blautia and that is not Dorea, Blautia, Dorea, at least one member of the order Clostridiales, Sutterella, Eubacterium, Collinsella, at least one member of the family Erysipelotrichaceae.

Embodiments of the invention encompass compositions, methods of producing the compositions and administration of the compositions where the compositions are intended for administration to canine patients (or subjects), and the compositions include, but are not limited to including, microorganisms of at least one of the following taxa: Dorea, at least one member of the order Clostridiales, Sutterella, Eubacterium, Collinsella, at least one member of the family Erysipelotrichaceae.

Embodiments of the invention encompass compositions, methods of producing the compositions and administration of the compositions where the compositions are intended for administration to canine patients (or subjects), and the compositions include, but are not limited to including, microorganisms of at least one of the following taxa: Dorea, at least one member of the order Clostridiales, Sutterella, Eubacterium, Collinsella, at least one member of the family Erysipelotrichaceae, Megamonas, at least one member of the family Ruminococcaceae, Clostridium, at least another member of the family Clostridiaceae, at least another member of the family Lachnospiraceae that is not Blautia and that is not Dorea, Faecalibacterium.

Embodiments of the invention encompass compositions, methods of producing the compositions and administration of the compositions where the compositions are intended for administration to canine patients (or subjects), and the compositions include, but are not limited to including, microorganisms, and the administration results in a significant change in at least one of the following microorganisms of the microbiota of the patient: Bacteroides, Enterococcus, Streptococcus, Ruminococcus, Blautia, Dorea, Prevotella, Clostridiaceae g1, Lachnospiraceae g1, Coprococcus, Oscillospira, Eubacterium, Clostridiales f1.

### BRIEF DESCRIPTION OF THE DRAWINGS

FIG. 1 is a flow chart for an exemplary embodiment of the invention.

FIG. 2 is a flow chart for another exemplary embodiment of the invention.

FIG. 3 is a flow chart for another exemplary embodiment of the invention.

FIG. 4 illustrates the proportion of microbes in healthy animals vs. treatment animals, both before and after treatment, in a limited evaluation of an embodiment of the present invention.

FIG. 5 illustrates an identification of most abundant taxa in an embodiment of the invention.

FIG. 6 is a bar chart comparing semi-successful or successful cases to the unsuccessful cases as a function of the feline study participant's age in an embodiment of the present invention.

FIG. 7 is a bar chart comparing semi-successful or successful cases to the unsuccessful cases as a function of the feline study participant's body condition in an embodiment of the present invention.

FIG. 8 illustrates microbial taxa that differed significantly (P<0.05) in relative abundance before and after treatment in domestic dogs receiving a full course of oral FMT capsules, an embodiment of the present invention.

FIG. 9 illustrates microbial taxa that differed significantly (P<0.05) in relative abundance before and after treatment in domestic cats receiving a full course of the oral FMT capsules, an embodiment of the present invention.

### DETAILED DESCRIPTION OF THE INVENTION

This patent describes compositions including microorganisms (such as but not limited to those occurring in a particular microbiota), and products and methods for therapeutic administration of microorganisms (such as but not limited to those occurring in a particular microbiota) to non-human animals, for example, to treat a disease or condition and/or to maintain health.

Embodiments of the invention encompass identification and characterization of microbiota having potential or demonstrated therapeutic effects for certain conditions in certain non-human animals. In some embodiments, the identification and characterization of microbiota may include identifying and characterizing the microbiota of potential donors, characterizing the potential donors, and identifying potentially therapeutic microbiota based on analysis of such data from multiple individuals. In some embodiments, the identification and characterization of microbiota may include testing, that is, administering microbiota of potential donors to animals suffering from a disease or condition, documenting any therapeutic response, and optionally identifying and characterizing the microbiota of recipients before and after treatment, and optionally identifying demonstrated therapeutic microbiota.

In some embodiments, therapeutic microbiota are identified based upon the identification and characterization of microbiota of potential donors, the characteristics of the potential donors, and the documented therapeutic responses of recipients including patients to administration of a microbiota.

Embodiments of the invention encompass identification and characterization of microbiota having therapeutic effects for certain diseases or conditions in certain species, groups or taxa of non-human animals. Conditions include, but are not limited to including, digestive, inflammatory, and other conditions, including both acute and chronic conditions. Non-limiting examples include colitis, constipation, acute or chronic diarrhea, gastritis, gastroenteritis, inflammatory bowel disease, irritable bowel disease, irritable bowel syndrome, pancreatitis, small intestinal malabsorption, vomiting and regurgitation, atopic dermatitis, obesity, and diabetes. Species, groups or taxa of non-human animals include, but are not limited to including, pets such as cats of various breeds, dogs of various breeds, rabbits, and ferrets, and livestock animals such as pigs, cow, horses, sheep, and goats. Further, these therapies may be of benefit to captive wildlife, such as, but not limited to, cheetahs.

Embodiments of the invention encompass products for administration of such microorganisms, including compositions for oral administration. Embodiments of the invention encompass treatment regimes, including dosing and time course of treatment.

Embodiments of the invention encompass compositions, methods of producing the compositions and administration of the compositions where the compositions include, but are not limited to including, microorganisms of at least one of the following taxa: Blautia, a member of Clostridiales, a member of Ruminococcaceae, Eubacterium, Faecalibacterium, Oscillospira, Phascolarctobacterium, a member of Mogibacteriaceae, a member of Succinivibrionaceae, Coprococcus, Roseburia, and Catenibacterium.

Embodiments of the invention encompass products for administration of such microorganisms, including compositions for oral administration. Embodiments of the invention encompass treatment regimes, including dosing and time course of treatment. Embodiments of the invention encompass compositions, methods of producing the compositions and administration of the compositions where the compositions include microorganisms of the following taxa: Blautia, a member of Clostridiales, a member of Ruminococcaceae, Eubacterium, Faecalibacterium, Oscillospira, Phascolarctobacterium, a member of Mogibacteriaceae, a member of Succinivibrionaceae, Coprococcus, Roseburia, and Catenibacterium. In some embodiments, for the compositions, the above-listed twelve taxa are the twelve most abundant.

Embodiments of the invention encompass compositions, methods of producing the compositions and administration of the compositions where the compositions are intended for administration to feline patients (or subjects), and the compositions include, but are not limited to including, microorganisms of at least one of the following taxa: Dorea, Ruminoccoccus, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella, Prevotella, Eubacterium, Oscillospira, at least another member of the family Clostridiaceae, at least another member of the family Lachnospiraceae that is not Blautia and that is not Dorea, Ruminococcus, Coprococcus, Parabacteroides, Fusobacterium, Faecalibacterium, Megamonas.

Embodiments of the invention encompass compositions, methods of producing the compositions and administration of the compositions where the compositions are intended for administration to feline patients (or subjects), and the compositions include, but are not limited to including, microorganisms of at least one of the following taxa: Dorea, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella.

Embodiments of the invention encompass compositions, methods of producing the compositions and administration of the compositions where the compositions are intended for administration to feline patients (or subjects), and the compositions include, but are not limited to including, microorganisms of at least one of the following taxa: at least one member of the order Clostridiales, at least one member of the family Ruminococcaceae, Clostridium, at least one member of the family Clostridiaceae, Sutterella.

Embodiments of the invention encompass compositions, methods of producing the compositions and administration of the compositions where the compositions are intended for administration to feline patients (or subjects), and the compositions include, but are not limited to including, microorganisms of at least one of the following taxa: at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, and Megamonas. Embodiments of the invention encompass compositions, methods of producing the compositions and administration of the compositions where the compositions are intended for administration to feline patients (or subjects), and the compositions include, but are not limited to including, microorganisms, and the administration results in a significant change in at least one of the following microorganisms of the microbiota of the patient: Blautia, Oscillospira, Ruminococcus, Lachnospiraceae g1, Clostridiales f1. Embodiments of the invention encompass compositions, methods of producing the compositions and administration of the compositions where the compositions are intended for administration to canine patients (or subjects), and the compositions include, but are not limited to including, microorganisms of at least one of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, at least one member of the family Erysipelotrichaceae, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium.

Embodiments of the invention encompass compositions, methods of producing the compositions and administration of the compositions where the compositions are intended for administration to canine patients (or subjects), and the compositions include, but are not limited to including, microorganisms of at least one of the following taxa: Ruminoccoccus, at least one member of the family Clostridiaceae, at least one member of the family Lachnospiraceae that is not Blautia and that is not Dorea, Blautia, Dorea, at least one member of the order Clostridiales, Sutterella, Eubacterium, Collinsella, at least one member of the family Erysipelotrichaceae.

Embodiments of the invention encompass compositions, methods of producing the compositions and administration of the compositions where the compositions are intended for administration to canine patients (or subjects), and the compositions include, but are not limited to including, microorganisms of at least one of the following taxa: Dorea, at least one member of the order Clostridiales, Sutterella, Eubacterium, Collinsella, at least one member of the family Erysipelotrichaceae.

Embodiments of the invention encompass compositions, methods of producing the compositions and administration of the compositions where the compositions are intended for administration to canine patients (or subjects), and the compositions include, but are not limited to including, microorganisms of at least one of the following taxa: Dorea, at least one member of the order Clostridiales, Sutterella, Eubacterium, Collinsella, at least one member of the family Erysipelotrichaceae, Megamonas, at least one member of the family Ruminococcaceae, Clostridium, at least another member of the family Clostridiaceae, at least another member of the family Lachnospiraceae that is not Blautia and that is not Dorea, Faecalibacterium.

Embodiments of the invention encompass compositions, methods of producing the compositions and administration of the compositions where the compositions are intended for administration to canine patients (or subjects), and the compositions include, but are not limited to including, microorganisms, and the administration results in a significant change in at least one of the following microorganisms of the microbiota of the patient: Bacteroides, Enterococcus, Streptococcus, Ruminococcus, Blautia, Dorea, Prevotella, Clostridiaceae g1, Lachnospiraceae g1, Coprococcus, Oscillospira, Eubacterium, Clostridiales f1.

### Definitions

The phrase “as used herein” encompasses all of the specification, the abstract, the drawings (figures), and the claims.

As used herein, the use of the singular herein includes the plural and vice versa unless expressly stated to be otherwise, or obvious from the context that such is not intended. That is, “a” and “the” refer to one or more of whatever the word modifies. For example, “a donor” may refer to one donor, two donors, etc. Likewise, “the product” may refer to one, two or more products. By the same token, words such as, without limitation, “products” would refer to one product as well as to a plurality of products unless it is expressly stated or obvious from the context that such is not intended.

As used herein, unless specifically defined otherwise, any words of approximation such as without limitation, “about,” “essentially,” “substantially,” and the like mean that the element so modified need not be exactly what is described but can vary from the description. The extent to which the description may vary will depend on how great a change can be instituted and have one of ordinary skill in the art recognize the modified version as still having the properties, characteristics and capabilities of the unmodified word or phrase. With the preceding discussion in mind, a numerical value herein that is modified by a word of approximation may vary from the stated value by ±15% in some embodiments, by ±10% in some embodiments, by ±5% in some embodiments, or in some embodiments, may be within the 95% confidence interval.

As used herein, all numbers that represent physical values or measurements are subject the standard deviation in the measurement of the value.

As used herein, any ranges presented are inclusive of the end-points. For example, “a temperature between 10° C. and 30° C.” or “a temperature from 10° C. to 30° C.” includes 10° C. and 30° C., as well as any temperature in between. In addition, throughout this disclosure, various aspects of this invention may be presented in a range format. The description in range format is merely for convenience and brevity and should not be construed as an inflexible limitation on the scope of the invention. Accordingly, the description of a range should be considered to have specifically disclosed all the possible subranges as well as individual numerical values within that range. As an example, a description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. Unless expressly indicated, or from the context clearly limited to integers, a description of a range such as from 1 to 6 should be considered to have specifically disclosed subranges 1.5 to 5.5, etc., and individual values such as 3.25, etc. that is non-integer individual values and ranges beginning with, ending with or both beginning with and ending with non-integer value(s). This applies regardless of the breadth of the range.

As used herein, a range may be expressed as from “about” one particular value and/or to “about” another particular value. When such a range is expressed, another embodiment is included, the embodiment being from one particular value and/or to the other particular value. Similarly when values are expressed as approximations by use of the antecedent “about,” it will be understood that the particular value forms another embodiment. As a non-limiting example, if “from about 1 to about 4” is disclosed, another embodiment is “from 1 to 4,” even if not expressly disclosed. Likewise, if one embodiment disclosed is a temperature of “about 30° C.,” then another embodiment is “30° C.,” even if not expressly disclosed. Similarly, numbers or ranges presented as a specific value or specific range also encompass another embodiment in which the number or the end of the range is preceded by “about.” As a non-limiting example, if “a temperature of 30° C.” is expressly disclosed, then another embodiment is “a temperature of about 30° C.,” even if not expressly disclosed. In a similar manner, if “from 1 to 4” is disclosed, another embodiment is “from about 1 to about 4,” even if not expressly disclosed.

As used herein, the use of “preferred,” “preferably,” or “more preferred,” and the like to modify an aspect of the invention refers to preferences as they existed at the time of filing of the patent application.

As used herein, “optional” means that the element modified by the term may or may not be present.

As used herein, the phrase “combination of” preceded by “a” or “any” and followed by a list joined by the conjunction “and” (and obvious variants of these), means any combination of two or more members of the group where the group members are the members of the list joined by the conjunction “and.” As a non-limiting example, “any combination of A, B, C, and D” encompasses the following combinations: A and B; A and C; A and D; B and C; B and D; C and D; A, B, and C; A, B, and D; A, C, and D; B, C, and D; A, B, C, and D. Similarly, for a list ending with or including “combinations thereof” (or obvious variants such as “all combinations thereof”), the above definition also applies. As a non-limiting example, the phrase “X is selected from the group consisting of A, B, C, D, and combinations thereof” means X is A, X is B, X is C, X is D, or X is “any combination of A, B, C, and D” where the above definition of “any combination thereof” applies. Likewise, a phrase such as “X is A, B, C, D, or a combination thereof” means X is A, X is B, X is C, X is D, or X is “a combination of A, B, C, and D” where the above definition of “a combination thereof” applies.

As used herein, the phrase “and/or” means a combination or an individual member. As a non-limiting example, “X is A, B, and/or C” encompasses the following possibilities: X is A; X is B; X is C; X is any combination of A, B, and C (A and B; A and C; B and C; A, B, and C).

As used herein, use of phrases “ . . . at least one of the following: A, B, C,” “ . . . at least one of the following: A, B, and C,” “ . . . at least one of the following: A, B, or C,” “ . . . at least one of the following [group, list, strain, etc.]: A, B, C,” “ . . . at least one of the following [group, list, strain, etc.]: A, B, and C,” “ . . . at least one of the following [group, list, strain, etc.]: A, B, or C,” mean that A alone, B alone, C alone, or any combination of A, B, and C satisfies the condition.

It does not mean “at least one A, at least one B, and at least one C” is required to satisfy the condition. Similar phrases with “at least two” would be interpreted similarly. If A, B, or C is a group or genus, “at least two,” “at least three,” and the like could be satisfied by two different members of group A. As a non-limiting example, “at least two of the following: halogen, OH, NH, CH₃, H,” is satisfied by the Cl and H, or Cl and I, or OH and H. On the other hand, “at least two of the following: Cl, halogen, OH, NH, CH₃, H,” is not met by Cl alone even though Cl is also a halogen, and thus falls into two groups, Cl and halogen, but would be met by Cl and I or F and I.

As used herein, the phrase “wt %” refers to percent by weight. As used herein, percent by weight will be used interchangeably with percent by mass.

As used herein, a “particle” may be a piece of matter of any shape held together by physical bonding of molecules, held together by chemical bonds, such as a cross-linked polymer network, held together by ionic interactions, an agglomeration of particles held together by colloidal forces and/or surface forces, or a piece of matter held together by any combination of agglomeration, surface forces, colloidal forces, ionic interactions, and chemical bonds. For the purposes of this disclosure, a particle may be defined as ranging in size from less than one tenth of a nanometer up to several centimeters in size. In addition, a particle may include one or more types of constituent molecules.

As used herein, “treatment of a disease and/or condition” includes, but is not limited to including, administration of a substance in an effective amount to a patient (animal including a human) suffering from a disease and/or condition, to have a beneficial effect on the health and well-being of the patient including at least one of the following (but not limited to including the following): (1) curing the disease or condition; (2) slowing the progress of the disease or condition; (3) causing the disease or condition to retrogress; and (4) alleviating one or more symptoms of the disease or condition.

As used herein, “prophylactic administration” includes, but is not limited to including, administration of a substance to a patient (animal, including a human), known or suspected of being particularly susceptible to a disease and/or condition, in a prophylactically effective amount to have a prophylactic beneficial effect on the health and well-being of the patient, which includes at least one of the following (but not limited to including the following): (1) preventing or delaying on-set of the disease or condition in the first place; (2) maintaining a disease or condition at a retrogressed level once such level has been achieved by administration of an effective amount of a substance for treatment, which may be the same as or different from the substance used in a prophylactically effective amount; and (3) preventing or delaying recurrence of the disease or condition after a course of treatment with an effective amount of a substance, which may be the same as or different from the substance used in a prophylactically effective amount, has concluded.

As used herein, “therapeutic administration,” with respect to administration of the microorganisms in the embodiments of the invention described herein, encompasses administration to maintain health, administration to treat a disease and/or condition, and prophylactic administration.

As used herein, “microbial strain” refers to a group of organisms characterized by a particular genetic variant or set of genetic variants, or a genetic subtype, of a microorganism For example, a “flu strain” is a certain genetic variant of the influenza or “flu” virus. A microbial strain may be cultured or may be naturally occurring.

### Methodologies

As noted above, embodiments of the invention encompass identification and characterization of microbiota associated with good health and/or having potentially or demonstrated therapeutic effects for certain diseases or conditions in certain non-human animals.

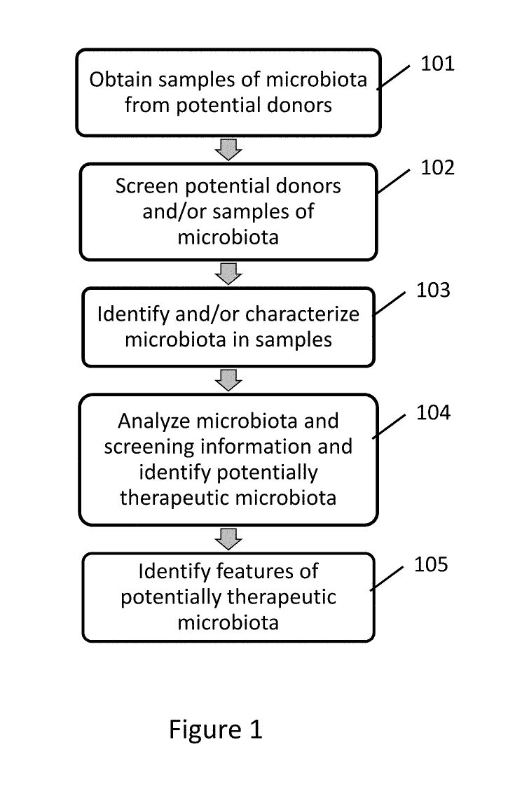

Such identification and characterization are accomplished in one embodiment by executing the following operations, as shown in FIG. 1: (i) Source microbiota samples from one or more potential donor animals 101; (ii) screen potential donors and/or microbiota samples from the potential donors for risk factors, such as from the animal's history or by testing for transmittable disease and/or pathogens 102; (iii) identify or otherwise characterize the microorganisms present in each of the microbiota samples 103; (iv) analyze the microbiota data and screening information, for example to exclude risky or atypical donors and/or samples, and identify potentially therapeutic donors and/or samples 104; and (v) optionally, analyze the data for potentially therapeutic donors and/or samples to identify common features of the potentially therapeutic microbiota 105.

In some embodiments, identification (iii) involves using sequencing methods known in the art to identify or otherwise characterize the microorganisms present in each of the microbiota sample. In some embodiments, the analysis (iv) involves identifying which taxa are present, and then determining whether a donor's profile correlates with the profiles of other healthy individuals in order to exclude “apparently healthy” individuals that may exhibit imbalances in the composition of the gut microbiome as potential donors. In some embodiments, the profiles of other healthy individuals are obtained from a database. In some embodiments, the analysis (v) involves testing for correlations of potential donors with those donors previously found to be therapeutic in order to identify common features of the potentially therapeutic microbiota.

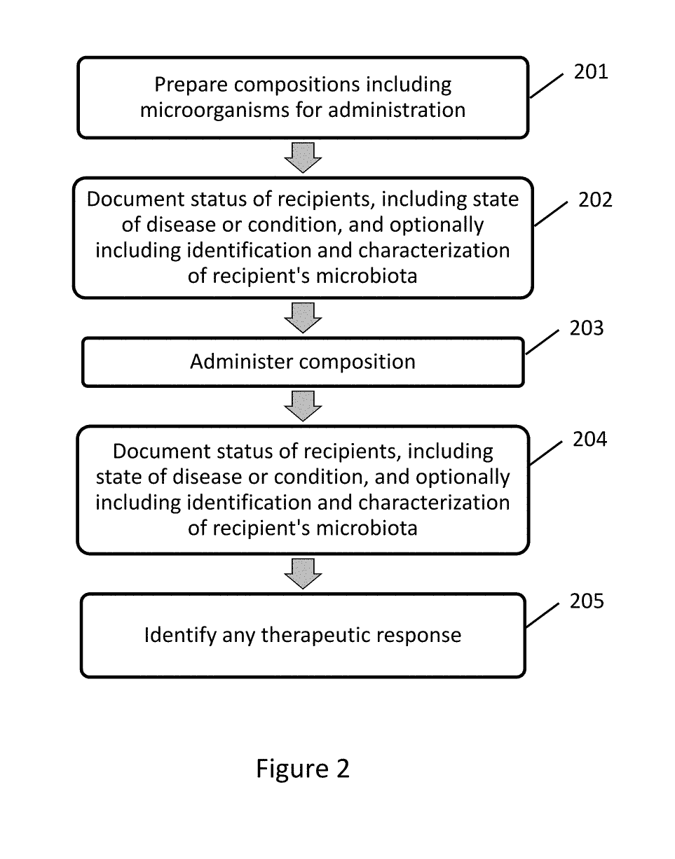

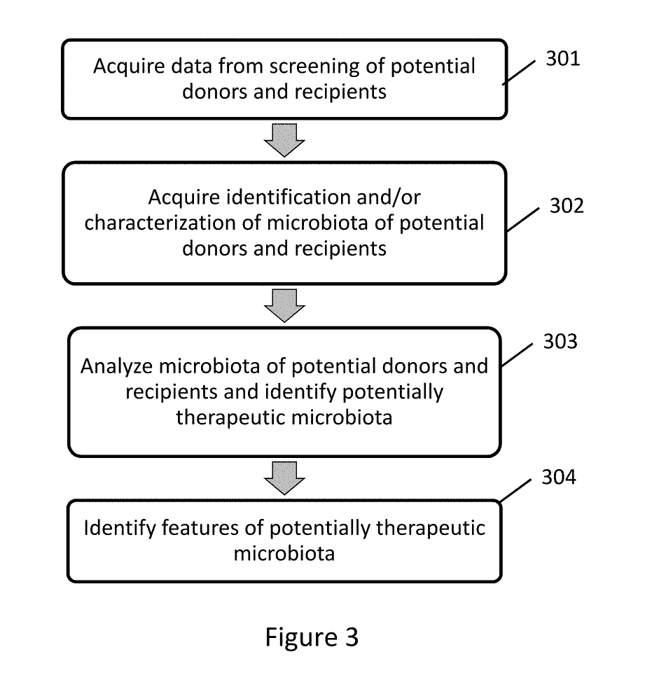

As noted above, embodiments of the invention also encompass identification and characterization of microbiota having demonstrated therapeutic effects for certain organisms and/or conditions in non-human animals. Such identification and characterization are accomplished in one embodiment by executing the following operations, as shown in FIG. 2: (i) Prepare or obtain compositions including potentially therapeutic microbiota for administration to animals, including animals having a particular disease or condition (“patients”) 201; (ii) document the state of potential recipients, including the presence or state of any disease or condition in recipients 202, and optionally including identifying and characterizing microbiota of the potential recipient; (iii) administer the prepared composition including microbiota to recipients, including one or more patients 203; (iv) document the state of the recipients after, and optionally during, receipt of treatment, including the state of the disease or condition, and optionally including identifying and characterizing microbiota of the recipients during and/or after treatment 204; and (v) identify any potentially therapeutic response based, at least in part, upon information about the state of the recipients before and after treatment 205. In some embodiments, the identification of any potentially therapeutic response is based on information about the state of the recipients before and after treatment such as, but not limited to, the frequency of diarrhea or vomiting, changes in body condition, body weight, and behavior. In another embodiment of the invention, information about therapeutic effects 205 identified from administration of a composition 201, as described for example with respect to FIG. 2, may be used in combination with information about microbiota that are potentially therapeutic 102, 103 to identify microbiota that are potentially therapeutic 104 and/or the features of potentially therapeutic microbiota 105. As a non-limiting example, identification and characterization are accomplished in one embodiment by executing the following operations, as shown in FIG. 3: (i) Acquire information about the screening of donors and recipients, including for example risk factors and/or state of disease or condition 301; (ii) acquire information characterizing the microorganisms present in microbiota of donors and recipients 302; (iii) analyze the microbiota data and screening information to consider risky or atypical donors or samples and the therapeutic response to receipt of compositions including known microbiota, and identify potentially therapeutic donors and/or samples 303; (iv) optionally, analyze the data for potentially therapeutic donors and/or samples to identify common features of the potentially therapeutic microbiota 304.

### Potential Donors and Recipients of Microorganisms

In the embodiments of the present invention, the donors and recipients of microorganisms, including microbiota, are non-human animals. In preferred embodiments, the donors and recipients are non-human mammals and the microorganisms are microbiota. In some embodiments, the donors and recipients are domestic mammals including both farm animals and companion animals. Examples of farm animals (livestock) include, but are not limited to including, cows, horses, donkeys, mules, pigs, sheep, and goats. Examples of companion mammals include, but are not limited to, cats, dogs, ferrets, rabbits, mice, gerbils, rats, hamsters, and guinea pigs. In some embodiments, the donors and recipients are only companion mammals.

In some embodiments, the donors and recipients are only cats, dogs, and ferrets. In some embodiments of the present invention, potential donors include “apparently healthy” donors. “Apparently healthy” donors are animals that do not exhibit health problems. In some embodiments of the present invention, qualification of an “apparently healthy” animal as a potential microbiota source (potential donor) includes determining that the animal does not exhibit any outwardly observable health problems based, for example, on examination of the animal and, optionally, on a review of the health history of the animal. In some embodiments of the present invention, qualification of an “apparently healthy” animal as a potential microbiota source includes assessing risk factors including, but not limited to, presence or absence of pathogens, living conditions, body condition, fecal consistency, frequency of vomiting and diarrhea, presence of skin and/or eye infections. Other information about the potential donor, such as breed, sex, and age, may also be used in assessing potential donors.

### Sourcing Microbiota

As used herein, “microbiota” refers to a community of microorganisms of a particular region. For example, the microbiota may be the community of microorganisms that exist in the gastrointestinal tract of an individual. Other non-limiting examples of regions include the ear, nose, throat, vaginal region, and skin of an individual. The microorganisms include at least bacteria, and may also include, but are not limited to including, viruses, fungi, yeast, or a combination thereof.

In some embodiments of the present invention, the microbiota is the microbiota of a sample obtained from a donor, for example, a sample of feces. “Feces” usually refers to matter discharged through an evacuation orifice such as the anus or cloaca. As used herein, the terms “feces,” “fecal matter,” “fecal material,” and “fecal sample” encompass solid, semisolid, or liquid metabolic waste excreted from an animal's digestive tract, and also including, for example, samples removed by a medical professional from an animal's digestive tract. It is understood that the microbiota samples obtained from donors may include other material in addition to the microbiota. For example, fecal samples may include undigested plant material such as cellulose, cholesterol, mucous secreted by the animal, minerals such as calcium phosphate and iron phosphate, protein, and bile pigments, etc.

### Screening

After sourcing microbiota samples from donors, there can be screening for pathogens and/or other risk factors. The risk factors include aspects of the donor and/or the microbiota sample that could make either or both an unsuitable source of microorganisms for a particular recipient and/or type of recipient. The risk factors may increase or decrease the likelihood that a recipient will benefit from receiving a microbiota sample from the donor and/or may present a risk of further health complications.

The risk factors include aspects of the donor and/or sample indicating, for example, disease and/or infection of the donor, such as the presence of a pathogen that could be passed to the recipient with treatment including the microbiota sample. The risk factors may include data indicating the donor is significantly different than the recipient, for example, because it is tolerant of a disease and/or condition of the recipient, or is not typical of the donor or recipient's species, breed, or taxon, for example, with respect to tolerance of a particular disease and/or condition. For example, a donor may have a microbiota that is suited to its particular tolerances or environment, such as its tolerance of a particular pathogen, diet, and/or stress level, but not suited to the tolerances or environment of the recipient.

In some embodiments, the microbiota samples, such as fecal samples, are screened for pathogens. In some embodiments, if the donors are cats, the pathogen screen includes at least screening for one or more of the following: Clostridium difficile toxins A and B, Cryptosporidium spp, Salmonella spp, feline coronavirus, feline parvovirus (Panleukopenia), Giardia spp, canine parvovirus 2, and Tritrichomonas foetus. In some embodiments, if the donors are dogs, the pathogen screen includes at least screening for one or more of the following: Clostridium difficile toxins A and B, Cryptosporidium spp, Salmonella spp, Giardia spp, canine parvovirus 2, Clostridium perfringens antigen, alpha toxin, and beta toxin. In some embodiments, if the donors are ferrets, the pathogen screen includes at least screening for one or more of the following: Cryptosporidium, Giardia spp., Canine distemper virus, Lawsonia, Campylobacter jejuni, Ferret coronavirus, Helicobacter spp, and Salmonella spp.

In some embodiments, the donors themselves are screened for transmittable disease and/or the presence of pathogens. The screening of the donors may include testing whole blood and/or plasma from the donor and/or execution of other medical evaluations. In some embodiments, the donors are screened for potential exposure to pathogens or harmful elements, for example, due to environmental exposure, treatment and/or care, and/or history such as living in an animal shelter or on a particular farm. In some embodiments, the donors are screened for factors that may provide tolerance to a disease or condition, such as a particular genetic profile and/or allele, presence of antibodies, evidence of past infection, and/or physiological condition. In some embodiments, screening criteria may also include some of the information collected on the potential donors such as breed, sex, and age.

In preferred embodiments, the microbiota sample is a fecal sample, the donor of the microbiota sample is screened at least for history of condition or disease, and the microbiota samples are screened at least for common pathogens.

In some embodiments, if the microbiota sample is a fecal material sample, the sample is also screened for consistency and those samples that are too firm or too soft are eliminated (these are samples with “poor fecal consistency”). A fecal sample that is too firm or too soft may result from intestinal inflammation. In some embodiments, the Bristol stool chart can be used to define fecal consistency. Based on the Bristol stool chart, feces of type 1 or 2 is hard and may be indicative of constipation, and feces of types 6 and 7 are very soft and indicative of intestinal inflammation. However, fecal form and consistency varies by species and consequently different stool charts are used for different species. Such charts for each species are known in the art.

### Identification and Characterization of Microbiota

After sourcing microbiota samples, the microorganisms present in each sample are identified. Identification includes identification of a particular microorganism by association to any taxonomic unit, such as family, genus, and/or species, or other recognized or meaningful group, as well as identification by other means, such as by analysis of genomic material for particular markers, sequences and/or other elements. In some embodiments, the microorganism identification describes the abundance and taxonomy of the microorganisms at the family level, and in some embodiments, at a level more refined than the family, such as genus, species and/or strain. In some embodiments, the microorganism identification describes the abundance and taxonomy of the microorganisms at different levels, with microorganisms identified at one or more of the family, genus, species, subspecies and/or strain levels.

In some embodiments, the identification of the microorganisms in the microbiota samples is accomplished by shotgun sequencing of extracted DNA and/or by targeting sequencing of bacterial diversity, for example (and without limitation), based on the V4 hypervariable region of 16S rRNA. After performing polymerase chain reaction (PCR) for such a marker gene, libraries may be sequenced, for example (and without limitation) using an Illumina MiSeq system, to generate 250 bp (base pair) paired-end amplicon reads, and the amplicon data may be multiplexed using dual barcode combinations for each sample. After sequencing, the samples may be demultiplexed, filtered based on quality scores (FASTQ), and chimeras removed. Binned sequence reads may then be characterized for taxonomic composition (number and abundance), for example (and without limitation), by using various software tools such as BLAST that compare sequence reads with reference libraries containing current taxonomic classifications and make de novo assignments for unidentified taxa. The embodiments of the present invention are not limited to the specific sequencing equipment and the specific software and databases described above.

In some embodiments, the microbiota may be further characterized, for example, based upon the abundance and/or similarity or relatedness of the identified taxonomic units. In some embodiments, the microbiota sample characterization includes alpha diversity measures such as species richness (number of taxa), species evenness (how close in number members of the community are), and the Shannon Diversity Index, which incorporates aspects of both richness and evenness.

In some embodiments, the microbiota may be characterized by the presence of one or more taxa having a particular abundance in subjects or the subjects in which it occurs. For example, the microbiota may be characterized by the presence of a taxon or taxa that is relatively abundant in the subjects in which it occurs, for example, constituting more than 10, 15, 20, 25 or 30% of the microbes in such subjects. Also for example, the microbiota may be characterized by the presence of a taxon or taxa that is moderately rare in the subjects in which it occurs, for example, constituting less than 6, 5, 4, 3, or 2% of the microbes in such subjects. Also for example, the microbiota may be characterized by the presence of a taxon or taxa that is relatively rare in the subjects in which it occurs, for example, constituting less than 2, 1, 0.5, 0.1, 0.05, 0.01% of the microbes in such subjects.

In some embodiments, the microbiota may be characterized by the presence of one or more taxa having a particular occurrence among subjects. For example, the microbiota may be characterized by the presence of a taxon or taxa that occur commonly among the microbiota or subjects, for example in more than 50, 60, 70, 75, 80, 90, 95, 97, 98, or 99% of subjects. Also for example, the microbiota may be characterized by the presence of a taxon or taxa that occur rarely among the microbiota or subjects, for example, in fewer than 25, 20, 10, 5, 4, 3, 2, or 1% of subjects.

In some embodiments, the microbiota may be characterized by both the abundance of microorganisms in a taxon or taxa and the occurrence of the taxon or taxa among subjects. For example, the microbiota may be characterized by the presence of a taxon or taxa having an abundance lower or higher than expected based upon the occurrence of the taxon or taxa among subjects, and the relationship between abundance and occurrence for other taxa occurring among the subjects.

In some embodiments, the microbiota may be characterized based upon comparison to the microbiota of one or more other donors. In some embodiments, the microbiota may be characterized based upon comparison to a database including the microbiota of two or more other donors. In some embodiments, the microbiota may be characterized based upon comparison to a compilation or database of at least 10 different donors. In some embodiments, the microbiota may be characterized based upon comparison to a compilation or database of at least 50 different donors.

The microorganism identification and/or characterization may be prior to, after, or concurrent with the screening of potential donors and/or samples. In some embodiments, the screening of the potential donors occurs at a different time than screening of the microbiota samples from the potential donors. In preferred embodiments, the microorganism identification is executed after or concurrently with screening of the microbiota sample and/or potential donors. Once the screening is complete, samples positive for one or more risk factors, as well as samples from donors where the donor is positive for one or more risk factors and/or otherwise not typical or representative of the recipient and/or group, may be excluded from further analysis or consideration. Alternatively, such samples and individuals may be included in further analysis but identified as not suitable for donation.

In one embodiment, a screen of microbiota samples for pathogens is completed prior to the initiation of the microorganism identification, and those samples positive for one or more pathogens, if any, are eliminated, and not analyzed for microorganism identification. In another embodiment, screening of microbiota for pathogens and/or screening of the potential donors for pathogens and transmittable disease is not complete prior to the initiation of the microorganism identification, and samples including one or more pathogens and/or samples from potential donors testing positive for pathogens and/or transmittable disease, are eliminated from analysis after the microorganism identification and characterization.

### Identification of Potentially Therapeutic Microbiota

In some embodiments, after the microorganism identification and any characterization of the samples are completed, an assessment as to whether the microbiota is a potentially therapeutic one is made.

In some embodiments, an analysis that may be accomplished using a computer program is done to group the taxonomic screening results.

In some embodiments, the assessment includes, but is not limited to including, an initial comparison of the microbiota data from the potential donors, and removal of those potential donors whose composition of microbiota reflects a potentially unhealthy state.

In some embodiments, the assessment includes, but is not limited to including, comparing the microbiota to a database including the microbiota of two or more other donors. In some embodiments, the assessment includes, but is not limited to including, comparing the microbiota to a compilation or database of at least 10 different donors. In some embodiments, the assessment includes, but is not limited to including, comparing the microbiota to donor microbiota that have been successfully used to treat a specific disease or condition in an animal (including human, but preferably a non-human animal).

### Identification of Demonstrated Therapeutic Microbiota

In some embodiments, the identification of therapeutic microbiota includes, but is not limited to including, preparing a composition of a potentially therapeutic microbiota or a portion of the microbiota for administration, administering a composition including potentially therapeutic microbiota to animals (recipients, e.g. study subjects), at least some of whom have symptoms of a health condition, such as diarrhea, and documenting and assessing its therapeutic effect, if any. FIG. 2 illustrates one embodiment for demonstration of the therapeutic effects of a particular microbiota or set of microorganisms in the treatment of a disease or condition in a non-human animal.

Assessing the therapeutic effect may include documenting symptoms of health and identifying microorganisms present in the subject both before and after treatment and optionally during treatment, and characterizing the response, if any. Typically, the identification of therapeutic microbiota involves evaluating efficacy of the administration of the composition in treating a disease or condition in a number of subjects suffering from the disease or condition. In some embodiments, the presence or absence of known pathogens before and after treatment may be used to identify therapeutic microbiota.

The assessment may be performed for a number of diseases in a number of different animal species. The inventors have found that some apparently healthy donors have microbiota that appears unhealthy. The inventors have found that some apparently healthy donors have a composition of microbiota that appears unhealthy or out of balance.

In some embodiments, the assessment includes, but is not limited to including, compiling data including information about the microbiota successfully used to treat a specific disease or condition in an animal (including human, but preferably a non-human animal), and identifying commonalities in the microbiota of those donors. In some embodiments, the assessment includes, but is not limited to including, analyzing information about the microbiota successfully used to treat a specific disease or condition in an animal (including human, but preferably a non-human animal), and identifying commonalities in the microbiota of those donors.

In some embodiments, the identification of commonalities includes but is not limited to including, identifying one or more microbial taxa each of which, when present, is relatively abundant in the microbiota successfully used to treat the specific disease or condition, for example, constituting more than 10, 15, 20, 25 or 30% of the microbes in such subjects. In some embodiments, the identification includes but is not limited to including, identifying one or more microbial taxa each of which, when present, is moderately rare in the subjects in which it occurs, for example, constituting less than 6, 5, 4, 3, or 2% of the microbes in such subjects. In some embodiments, the identification includes but is not limited to including, identifying one or more microbial taxa each of which, when present, is relatively rare in abundance in the microbiota successfully used to treat the specific disease or condition, for example, constituting less than 2, 1, 0.5, 0.1, 0.05, or 0.01% of the microbes in such subjects.

In some embodiments, the identification includes but it not limited to including, identifying one or more microbial taxa that occur commonly among the microbiota successfully used to treat the specific disease or condition, for example, in more than 50, 60, 70, 75, 80, 90, 95, 97, 98, or 99% of the microbiota or subjects. In some embodiments, the identification includes but it not limited to including, identifying one or more microbial taxa that occur rarely among the microbiota successfully used to treat the specific disease or condition, for example, in fewer than 25, 20, 10, 5, 4, 3, 2, or 1% of subjects.

In some embodiments, the identification may be based upon both the abundance of microorganisms in a taxon or taxa and the occurrence of the taxon or taxa among microbiota or subjects. For example, in some embodiments, the identification includes, but is not limited to including, identifying one or more microbial taxa each of which, when present, is relatively abundant in the microbiota successfully used to treat the specific disease or condition and also occurs commonly among the microbiota successfully used to treat the specific disease or condition. In some embodiments, the identification includes but is not limited to including, identifying one or more microbial taxa each of which, when present, is relatively rare in abundance in the microbiota successfully used to treat the specific disease or condition and also occurs commonly among the microbiota successfully used to treat the specific disease or condition.

Also for example, the microbiota may be characterized by the presence of a taxon or taxa having an abundance lower or higher than expected based upon the occurrence of the taxon or taxa among subjects, considering, for example, the relationship between abundance and occurrence for other taxa occurring in the microbiotas of the subjects.

### Microorganisms for Compositions

A composition including microorganisms is made for administration of microorganisms to a recipient for therapy as well as for testing and evaluation.

In one embodiment, the composition is the fecal matter itself, which may be cleaned of outside contamination, such as, but not limited to, cat litter, and then processed, and thus the microorganisms are the ones that survive processing. In another embodiment, the composition includes, and may be limited to, microorganisms derived from cultures. In another embodiment, the composition includes, and may be limited to, microorganisms obtained from fermentation. In another embodiment, the composition includes, and may be limited to, microorganisms obtained from fermentation of microorganisms from freeze-dried fecal material. In some embodiments, the composition includes, but is not limited to, the microorganisms of a fecal material sample.

For example, the composition may include microorganisms derived from a fecal matter sample and microorganisms derived from cultures. In some embodiments, the composition includes, but is not limited to, the fecal microbiota of a donor, or substantially the fecal microbiota of a donor.

As used herein, “substantially the fecal microbiota of a donor” and “substantially a fecal microbiota” mean at least one of the following criteria are met: a) a substantial portion of the identifiable taxonomic units; b) presence of the most abundant microbial taxa in a sample, for example, presence of the three most common taxon or the most common taxa that account for 70% of the microorganisms in the sample; c) the portion of the microbiota that is still viable after processing for administration, where processing includes, but is not limited to, freezing and thawing, freeze-drying, and/or spray-drying. It is understood that processing is not carried out aseptically and incidental contamination with microorganisms in the environment occurs. In some embodiments, the microbiota samples, for example, a fecal sample, may be separated or purified by process such as centrifugation, celltrifugation, filtration, plasmapheresis, as well as other processes.

In some embodiments, the microorganisms of the composition are one or more cultured microbial taxa and/or strains. In some embodiments, the microorganisms of the composition are one or more isolated microbial taxa and/or strains. An isolated microbial taxon or strain, or isolated microbial taxa and/or strains, is/are a microorganism(s) isolated from the microbiota in which it/they normally occurs/occur. In other words, one or more microbial taxa or strains are separated from a microbiota sample. In some embodiments, the microorganisms of the composition are obtained from a microbiota of a donor and one or more of the microbial strains and/or taxa that have been enhanced. A microbial taxon and/or strain in a microbiota of a donor may be enhanced by adding a food/substrate (or adding more of a food and/or substrate) particularly suited to the microbial taxon or strain such that the microbes of the taxon and/or strain grow at a greater rate than the remaining microorganisms.

In another embodiment, the composition includes, and may be limited to, microorganisms obtained from fermentation. In another embodiment, the composition includes, and may be limited to, microorganisms obtained from fermentation of microorganisms from freeze-dried fecal material. In some embodiments, the microorganisms are obtained from fermentation of microorganisms from freeze-dried fecal material where one or more microbial taxa and/or strains have been isolated from the fecal material prior to freeze-drying and/or prior to fermentation. In some embodiments, the microorganisms are obtained from fermentation of microorganisms from freeze-dried fecal material where one or more microbial taxa and/or strains have been removed from the fecal material prior to freeze-drying and/or prior to fermentation and the remaining microbial taxa and/or strains are the ones used for fermentation.

In some embodiments, the microorganisms of the composition are a combination of the above. As non-limiting examples, the microorganisms of the composition may be a microbiota of a donor, substantially the microbiota of a donor, or a microbiota of a donor with one or more enhanced microbial strains, with one or more cultured microbial strains added, with one or more isolated microbial strains added, or with both one or more cultured microbial strains added and one or more isolated microbial strains added. Any of the above compositions may also include one or more taxa and/or strains obtained from fermentation. In some embodiments, the compositions including microorganisms expressly exclude a mixture of the microbiota of two or more donors.

Fermentation involves growing either undefined or defined microbial communities of bacteria, fungi and other organisms in culture fluid inside a bioreactor under controlled growth conditions, such as temperature, nutrient concentrations, pH, and dissolved gases.

In some embodiments, the microorganisms of the composition include, but are not limited to including, a combination of bacterial taxa and/or strains including members of the following taxa: Blautia, Catenibacterium, Coprococcus, Dorea, Eubacterium, Faecalibacterium, Oscillospira, Phascolarctobacterium, Mogibacteriaceae, Roseburia, and Succinivibrionaceae. In some embodiments, the microorganisms of the composition intended for administration to a cat, include, but are not limited to including, a combination of bacterial taxa and/or strains including members of the following taxa: Blautia, Catenibacterium, Coprococcus, Dorea, Eubacterium, Faecalibacterium, Oscillospira, Phascolarctobacterium, Mogibacteriaceae, Roseburia, and Succinivibrionaceae. In some embodiments, for the compositions, the above-listed twelve taxa are the twelve most abundant for the microorganisms of the composition. In some embodiments, for the compositions, the above-listed twelve taxa are the twelve most abundant of the microorganisms used for preparation of the composition.

In some embodiments, the microorganisms of the composition include, but are not limited to including, a combination of bacterial taxa and/or strains including members of the following taxa: a significantly higher proportion of Lachnospiraceae Blautia. In some embodiments, the microorganisms of the composition, intended for administration to a cat, include, but are not limited to including, a combination of bacterial strains including members of the following taxa: a significantly higher proportion of Lachnospiraceae Blautia. In some embodiments, higher means more, or at least one standard deviation more, than average of healthy subjects. In some embodiments, higher means higher than the level in the subject or patient.

In some embodiments, the microorganisms of the composition include, but are not limited to including, a combination of microorganisms from the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, Megamonas, at least one member of the family Ruminococcaceae, Clostridium, Prevotella, at least another member of the family Clostridiaceae, at least another member of the family Lachnospiraceae that is not Blautia and that is not Dorea, and Faecalibacterium. In some embodiments, the microorganisms of the composition include, but are not limited to including, microorganisms of at least one of the following taxa: Dorea, Ruminoccoccus, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella, Prevotella, Eubacterium, Oscillospira, at least another member of the family Clostridiaceae, at least another member of the family Lachnospiraceae that is not Blautia and that is not Dorea, Ruminococcus, Coprococcus, Parabacteroides, Fusobacterium, Faecalibacterium, Megamonas. In some embodiments, the microorganisms of the composition include, but are not limited to including, microorganisms of at least two, at least three, at least four, at least five, at least six, at least seven, at least eight, and/or at least ten of the following taxa: Dorea, Ruminoccoccus, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella, Prevotella, Eubacterium, Oscillospira, at least another member of the family Clostridiaceae, at least another member of the family Lachnospiraceae that is not Blautia and that is not Dorea, Ruminococcus, Coprococcus, Parabacteroides, Fusobacterium, Faecalibacterium, Megamonas.

In some embodiments, the microorganisms of the composition include, but are not limited to including, microorganisms of at least one of the following taxa: Dorea, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella. In some embodiments, the microorganisms of the composition include, but are not limited to including, at least two, at least three, at least four, at least five, at least six, at least seven, at least eight, at least nine, at least ten, and/or at least twelve of the of the following taxa: Dorea, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella.

In some embodiments, the microorganisms of the composition include, but are not limited to including, microorganisms of at least one of the following taxa: at least one member of the order Clostridiales, at least one member of the family Ruminococcaceae, Clostridium, at least one member of the family Clostridiaceae, Sutterella. In some embodiments, the microorganisms of the composition include, but are not limited to including, at least two, at least three, at least four, at least five, at least six, at least seven, at least eight, at least nine, at least ten, and/or at least twelve of the of the following taxa: at least one member of the order Clostridiales, at least one member of the family Ruminococcaceae, Clostridium, at least one member of the family Clostridiaceae, Sutterella.

In some embodiments, the microorganisms of the composition include, but are not limited to including, microorganisms of at least one of the following taxa: at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, and Megamonas. In some embodiments, the microorganisms of the composition include, but are not limited to including, microorganisms of at least two, at least three, at least four, at least five, at least six, at least seven, and/or at least eight of the following taxa: at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, and Megamonas.

In some embodiments, the microorganisms of the composition include, but are not limited to including, microorganisms of at least one of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, at least one member of the family Erysipelotrichaceae, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium. In some embodiments, the microorganisms of the composition include, but are not limited to including, microorganisms of at least two, at least three, at least four, at least five, at least six, at least seven, at least eight, at least nine, and/or at least ten of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, at least one member of the family Erysipelotrichaceae, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium.

In some embodiments, the microorganisms of the composition include, but are not limited to including, microorganisms of at least one of the following taxa: Ruminoccoccus, at least one member of the family Clostridiaceae, at least one member of the family Lachnospiraceae that is not Blautia and that is not Dorea, Blautia, Dorea, at least one member of the order Clostridiales, Sutterella, Eubacterium, Collinsella, at least one member of the family Erysipelotrichaceae. In some embodiments, the microorganisms of the composition include, but are not limited to including, microorganisms of at least two, at least three, at least four, at least five, at least six, and/or at least seven of the of the following taxa: Ruminoccoccus, at least one member of the family Clostridiaceae, at least one member of the family Lachnospiraceae that is not Blautia and that is not Dorea, Blautia, Dorea, at least one member of the order Clostridiales, Sutterella, Eubacterium, Collinsella, at least one member of the family Erysipelotrichaceae.

In some embodiments, the microorganisms of the composition include, but are not limited to including, microorganisms of at least one of the following taxa: Dorea, at least one member of the order Clostridiales, Sutterella, Eubacterium, Collinsella, at least one member of the family Erysipelotrichaceae. In some embodiments, the microorganisms of the composition include, but are not limited to including, microorganisms of at least two, at least three, at least four, at least five, at least six, and/or at least seven of the of the following taxa: Dorea, at least one member of the order Clostridiales, Sutterella, Eubacterium, Collinsella, at least one member of the family Erysipelotrichaceae.

In some embodiments, the microorganisms of the composition include, but are not limited to including, microorganisms of at least one of the following taxa: Dorea, at least one member of the order Clostridiales, Sutterella, Eubacterium, Collinsella, at least one member of the family Erysipelotrichaceae, Megamonas, at least one member of the family Ruminococcaceae, Clostridium, at least another member of the family Clostridiaceae, at least another member of the family Lachnospiraceae that is not Blautia and that is not Dorea, Faecalibacterium. In some embodiments, the microorganisms of the composition include, but are not limited to including, microorganisms of at least two, at least three, at least four, at least five, at least six, and/or at least seven of the of the following taxa: Dorea, at least one member of the order Clostridiales, Sutterella, Eubacterium, Collinsella, at least one member of the family Erysipelotrichaceae, Megamonas, at least one member of the family Ruminococcaceae, Clostridium, at least another member of the family Clostridiaceae, at least another member of the family Lachnospiraceae that is not Blautia and that is not Dorea, Faecalibacterium.

### Methods for and Preparations of Compositions

The compositions including microorganisms for therapeutic administration (and for evaluation of potential donors), also referred to as dosage forms, include any pharmaceutical dosage form suitable for enteral administration. Enteral administration is administration within or by way of the gastrointestinal tract, also known as the alimentary canal. Enteral administration includes, without limitation, oral, buccal, sublingual, and rectal administration.

Oral administration is administration into the mouth or administration into the mouth with swallowing. Oral administration includes, without limitation, the administration of solid oral dosage forms, liquid dosage forms, gels, pastes, sprays, or any combination thereof. Solid oral dosage forms include, without limitation, capsules, both hard shell and soft shell, tablets, pills, powders, and granules. Liquid dosage forms for oral administration include, without limitation, emulsions, solutions, suspensions, syrups and elixirs. Granules or powders may be reconstituted as an oral suspension or solution for administration.

Buccal administration is administration by absorption into the gum, into the check, or both. Sublingual administration is by placement of the dosage form under the tongue. Buccal and sublingual administration are typically accomplished using a solid oral dosage form, or gel. As a non-limiting example, buccal and/or sublingual administration may be used for administration of microorganisms from the mouth of a donor.

Rectal administration may be by administration of a solid oral dosage form, by administration of a semi-solid form such as a suppository, gel, or ointment, by administration of a liquid, or by administration of both a semi-solid and a liquid. Administration of liquids such as solutions, emulsions, dispersions, or combinations thereof may be accomplished with an enema.

In preferred embodiments, the compositions including microorganisms are solid compositions for oral administration (also known as solid oral dosage forms). In some embodiments, the solid oral dosage forms are stable at room temperature (about 20° C. to about 25° C.). In some embodiments, the solid oral dosage forms are stable at room temperature (about 20° C. to about 25° C.) for a period of at three months. In some embodiments, the solid oral dosage forms are stable at room temperature (about 20° C. to about 25° C., at a relative humidity in the range of 30% to 65%) for a period of at least three months, or a period of at least six months. In order to form a solid from the microorganisms, the microorganisms are frozen, freeze-dried, lyophilized, spray-dried, or a combination thereof, and/or otherwise formed into a solid and/or fixed onto a solid. The processes of freezing, freeze-drying, lyophilizing, and spray-drying are known to those of skill in the art.

Prior to processing the microorganisms or the sample including the microorganisms—such as freezing, freeze-drying, lyophilizing, spray-drying, or a combination thereof—one or more excipients may be added to the microorganisms or the sample including the microorganisms to form an intermediate composition. The excipients disclosed herein may be used individually, or in combination with one or more other excipients, including, but not limited to, those described herein. In some embodiments, the excipients used are food grade substances as determined by the United States Food and Drug Administration (USFDA), pharmaceutical grade substances, or substances complying with a standard in the Foods Chemicals Codex (FCC), United States Pharmacopeia and/or National Formulary (USP/NF), the British Pharmacopeia, European Pharmacopeia, Japanese Pharmacopeia, or a combination thereof.

As used herein, an “excipient” may be a substance that is combined with the microorganisms to form a final dosage form. Excipients are non-toxic, and are typically inert. In other words, an excipient itself is typically not a drug. Excipients typically perform a function such as acting as a binder for the microorganisms, a carrier or a diluent for the microorganisms, a permeation enhancer, and/or an antioxidant and/or stabilizer for the microorganisms. In some cases vitamins and/or minerals and/or drugs, which may be used for therapeutic administration themselves, may also be an excipient. Unlike a solvent, which may be removed from the final dosage form, an excipient is not removed, but remains part of the final dosage form.

As used herein, a “drug” refers to a substance, other than the microorganisms of the compositions described herein, which when used in an effective amount may be used in treatment of disease and/or condition, and/or when used in a prophylactically effective amount may be used for prophylactic administration. In addition, a “drug” also refers to pharmaceutically acceptable, pharmacologically active derivatives of those drugs specifically mentioned herein, including, but not limited to, salts, esters, amides, and the like. In some embodiments, a “drug” also refers to a substance useful for diagnostics. In some embodiments, a “drug” does not include a substance useful only for diagnostics.

As used herein, a “solvent” may be a substance capable of dissolving, partially dissolving, dispersing, or suspending one or more substances to form a uniform dispersion and/or solution, with or without agitation, at a selected temperature and pressure. The substance may be a liquid, a gas, or a supercritical fluid. Also, a “solvent” may be a substance capable of partially dissolving, and dispersing and/or suspending one or more substances to form a uniform dispersion and/or solution, with or without agitation, at a selected temperature and pressure. The substance may be a liquid, a gas, or a supercritical fluid. A solvent herein may be a blend of two or more such substances. In some embodiments, a solvent may be used as a processing aid in forming a dosage form, but is removed, or substantially removed, during processing and does not form part of the final dosage form (except for incidental residual solvent). Some substances may be a solvent in some cases, and an excipient in other cases. As an example, water may be a carrier (an excipient) in a liquid dosage form, such as a suspension, but may be a solvent when used to prepare freeze-dried powders. In some embodiments, a substance used as an excipient in a dosage form is not a solvent even if it is capable of dissolving, partially dissolving, dispersing, or suspending one or more substances to form a uniform dispersion and/or solution.

If the microorganisms, or an intermediate composition including the microorganisms (such as and without limitation a fecal sample) is to be freeze-dried or lyophilized, an excipient may be added, and the excipient may be a cryoprotectant. A cryoprotectant is a substance that can help preserve viability of biological cells during freezing, storage, and thawing. Examples of cryoprotectants include, but are not limited to, glycol, glycerol, vegetable glycerol, dimethylsulfoxide, 1,3-propanediol, ethanol, polyvinyl alcohol, propylene glycol, trimethylene glycol, diethylene glycol, polyethylene glycol, polypropylene glycol, polyethylene oxide, mannitol, D-Mannitol, D-Sorbitol, sorbitol, dulcitol, xylitol, glucose, D-Glucose, xylose, sucrose, lactose, maltose, trehalose, raffinose, dextran, mannan, dextrin, FICOLL® (a high molecular weight polysaccharide that dissolves in water), gum arabic (acacia), acetamide, methylacetamide, dimethylformamide, dimethyl-acetamide, succinimide, methylpyrrolidone, polyvinylpyrrolidone, proline, glycine, glutamic acid, aminobutyric acid, glutaric acid, ammonium acetate, cysteines, EDTA (ethylenediamine tetra-acetic acid), blood serum, fetal calf serum, albumins, bovine serum albumin (BSA), gelatin, casein hydrolysate, starch hydolysate, hydroxypropyl cellulose, methylcellulose, ethylcellulose, hydroxypropyl methylcellulose, peptones, shell extract, glycoproteins, mucin, valinomycin, gramicidin, yeast extract, malt extract, skim milk, dairy milk, soy milk, fish oil, honey, polysorbate 80, polysorbate 20, polysorbate 60, polyethylene glycol p-(1,1,3,3-tetramethylbutyl)-phenyl ether (Triton® X-100), macrocyclon (C17H2803), glycine betaine, and combinations thereof. If the microorganisms, or an intermediate composition including the microorganisms (such as and without limitation a fecal sample), is to be spray dried, an excipient may also be included in the solution that is spray dried. Non-limiting examples of the type of excipients that may be added include starch, biodegradable polymers, and natural polymers.

In some embodiments in which an excipient is added, the weight to weight ratio of the excipient to the microorganisms, and/or the weight to weight ratio of the excipient to an intermediate composition including the microorganisms may range from about 1:100 to about 100:1, preferably from about 2:1 to about 20:1, and even more preferably from about 1:1 to about 1:4. In some embodiments in which an excipient is added, the weight to weight ratio of the excipient to the microorganisms, and/or the weight to weight ratio of the excipient to an intermediate composition including the microorganisms, before freeze-drying, spray-drying, or the like, may range may range from about 1:100 to about 100:1, preferably from about 2:1 to about 20:1, and even more preferably from about 1:1 to about 1:4. In some embodiments, the weight to weight ratio is a ratio of the excipient to the fecal matter and the ratio may range from about 1:100 to about 100:1, preferably from about 2:1 to about 20:1, and even more preferably from about 1:1 to about 1:4. In some embodiments, the weight to weight ratio is a ratio of the excipient to the fecal matter before freeze-drying, spray-drying, or the like, and the ratio may range from about 1:100 to about 100:1, preferably from about 2:1 to about 20:1, and even more preferably from about 1:1 to about 1:4.

It is also understood that the weight percent (wt %), for the compositions, is determined by the quantity of the materials added to the composition. Thus, the calculated % ingredient can also include impurities, moisture, residual solvents (from manufacture), or a combination thereof included with the ingredient as added to the composition. As a not-limiting example, if 20 grams of glycerol is added to 80 grams of a fecal material, the glycerol wt % is 100*(20/(20+80))=20 wt % of the total composition (or the weight of glycerol added is 25% of the weight of the fecal material (100*(20/80)=25%), even if the glycerol has some moisture, impurities, etc.

If the frozen, freeze-dried, lyophilized, and/or spray-dried composition including the microorganisms is of a particle size that is too large, it may be subject to one or more operations to reduce the particle size. The reduction of the particle size may be accomplished by crushing, grinding, cutting, and/or milling. Other techniques may be used instead of or in addition to those listed. The final particle size is a size sufficient for the intended use such as and without limitation filling a hard gelatin capsule. In some embodiments, the particles are of a size such that 95 wt % of the particles pass through a size 16 mesh U.S. sieve and are retained on a size 200 mesh U.S. sieve.

In some embodiments, the dosage form may be in the form of beads or micro-particles that include the microorganisms. For example, the microorganisms, or the microorganisms in combination with one or more excipients, may be encapsulated in a polymer to form micro-particles. Some non-limiting examples of the manufacture of nano-particles and micro-particles are found in United States Patent Application No. US 2004/0009229 A1, published on Jan. 15, 2004, and United States Patent Application No. US 2006/0002971 A1, published on Jan. 5, 2006. Non-limiting examples of bead dosage forms are found in U.S. Pat. Nos. 4,524,060, 5,026,560, and 6,224,910.

In preferred embodiments, the composition including the microorganisms is processed into a solid powder. The powder may be filled into a capsule or compressed into a tablet, or the powder may be used “as is” or some combination thereof. For capsules, the powder may be used alone to fill the capsule, or the powder may be combined with one or more excipients before being filled into the capsule. Excipients typically used in capsules are known to those of skill in the art. If the powder is compressed into a tablet, it may be compressed by itself, or combined with one or more excipients, such as, and without limitation, disintegrants, lubricants, fillers, stabilizers, diluents, and binders. The size of capsule (or size of tablet) used will depend upon the species of non-human animal donor and recipient. As non-limiting examples, a size 4 capsule may be used for cats and very small dogs, and a size 0 capsule for medium to large dogs.

In some embodiments, nutrients and/or other growth factors are added to the compositions to encourage growth of the microorganisms.

In preferred embodiments, the tablets, capsules, and/or other solid dosage forms are coated with an enteric coating that does not dissolve in the pH of the stomach but will dissolve in the intestine. There are different types of enteric coatings, which dissolve in different pH ranges. If the microorganisms are included in beads, micro-particles, and/or nano-particles, the beads, micro-particles, and/or nano-particles may be enteric coated. The enteric coated beads, micro-particles, and/or nano-particles may be filled into capsules, with or without one or more excipients, or may be later reconstituted as a suspension in water. Non-limiting examples of enteric coatings include, but are not limited to, edible shellac, shellac, methacrylic acid copolymers, cellulose acetate phthalate, hydroxypropylmethyl cellulose phthalate, polyvinyl acetate phthalate, carboxymethylethylcellulose, EUDRAGIT®-type polymer (poly(methacrylic acid, methylmethacrylate), hydroxypropyl methylcellulose acetate succinate, cellulose acetate trimellitate, and other suitable enteric coating polymers. The EUDRAGIT®-type polymers include, for example, EUDRAGIT® FS 30D, L 30 D-55, L 100-55, L 100, L 12.5, L 12.5 P, RL 30 D, RL PO, RL 100, RL 12.5, RS 30 D, RS PO, RS 100, RS 12.5, NE 30 D, NE 40 D, NM 30 D, S 100, S 12.5, and S 12.5 P. The enteric coating materials disclosed herein may be used individually, or in combination with one or more other enteric coatings, including, but not limited to, those enteric coating materials described herein. Enteric coating materials may include other excipients. A base coat or primer coat may be applied prior to the application of the enteric coating. Tablet and capsule coatings both enteric and non-enteric are known to those of skill in the art. A color coat or another type of coating may be applied on top of the enteric coating.

In some embodiments, the compositions including microorganisms also include, but are not limited to including, fiber. Fiber may be in the form of one or more oligosaccharides, which includes but is not limited to, fructans (fructooligosaccharides and inulins) and galactans (galactooligosaccharides). Fiber may also be in the form of resistant starch, pectin, one or more beta-glucans and/or one or more xylooligosaccharides. Resistant starch is a starch that is not digested or not completely digested in the intestinal tract of an animal (including human). Sources of fiber may be used individually or in combination with other sources of fiber, including, but not limited to including, those specifically described herein. Commercial sources of fiber suitable for oral consumption are known to those of skill in the art. Some excipients typically used in tablets and capsules may also be “fiber.” One of skill in the art will be able to determine if a particular compound is being added as “fiber” or performs some other function, or both. In some embodiments, a compound or excipient may be “fiber,” and also perform an additional function, such as acting as a filler. In some embodiments, the fiber may be a carrier of the microorganisms. In some embodiments, the fiber used may be food grade substances as determined by the United States Food and Drug Administration (USFDA), pharmaceutical grade substances, or substances complying with a standard in the Foods Chemicals Codex (FCC), United States Pharmacopeia and/or National Formulary (USP/NF), the British Pharmacopeia, European Pharmacopeia, Japanese Pharmacopeia, or a combination thereof.

The fiber may be added to the composition including microorganisms prior to processing by freeze-drying, spray-drying, etc. As a non-limiting example, the weight to weight ratio of the fiber to the microorganisms, and/or the weight to weight ratio of the fiber to an intermediate composition including the microorganisms (such as but not limited to, fecal matter) may range from about 1:100 to about 100:1, preferably from about 20:1 to about 20:1, and even more preferably from about 1:1 to about 1:4. The ranges above may be in addition to one or more excipients. In some embodiments, the above ranges apply to a combination of the fiber and one or more excipients with the weight ratio of fiber to excipient(s) being from about 1:50 to about 50:1, preferably about 1:20 to about 20:1.

In some embodiments, fiber is combined with processed microorganisms, such as but not limited to a powder, micro-particles, nano-particles, and/or beads, and filled into a capsule, compressed into a tablet, and/or filled into a bottle, package and/or sachet. In some embodiments, the microorganisms are included in beads, micro-particles, and/or nano-particles, which may be enteric coated. In some embodiments, a blend including, but not limited to including, fiber and microorganisms (whether in the form a powder, micro-particles, nano-particles, and/or beads) are later reconsistuted as a suspension in water or an aqueous fluid and/or directly added to food. In some embodiments, a blend including, but not limited to including, fiber and microorganisms (whether in the form of a powder, micro-particles, nano-particles, and/or beads) may be packaged in a bottle with multiple doses, or a small package, bottle, or sachet for one-time use (or individual use, or unit dosage).

In preferred embodiments, the microorganisms are processed, optionally with one or more excipients, to form a solid powder, which is blended or mixed with fiber, and then filled into hard capsules, and preferably, the filled capsules are subsequently enteric coated. In some embodiments, the blend of solid powder including microorganisms with fiber may be compressed into a tablet, and in some embodiments, the tablet is enteric coated.

The weight ratio (or mass ratio) of powder, micro-particles, nano-particles, and/or beads including microorganisms, to fiber used to fill the capsules (or compress into a tablet or used as a blend described above) may be about 1:100 to about 100:1, preferably from about 20:1 to about 20:1, and even more preferably from about 1:1 to about 1:5. In addition to the fiber, the blend used to fill the capsules and/or which is compressed into a tablet may include one or more other excipients. In preferred embodiments, the one or more excipients is 0.1% to 30 wt % of the final blend. As a non-limiting example, if excipient(s) comprise 20 wt % of the blend, and the weight ratio of powder including microorganisms to fiber is 1:4, the final composition is 20 wt % excipient(s), 16% powder including microorganisms, and 64% fiber.

In some embodiments, the number of capsules or tablets (or quantity of the composition including the microorganisms) may be higher or greater with the fiber added to the formulation than the number of capsules or tablets (or quantity of the composition including the microorganisms) than without the addition of fiber. As a non-limiting example, if no fiber is added and about 20-25 wt % of the capsule filler is excipient(s), one size 4 capsule may be sufficient for a dose for a cat, but with fiber added, two or three size 4 capsules may be needed per each administration.

In some embodiments, a solid powder, beads, and/or micro-particles including, but not limited to including the microorganisms are added to food, water, and/or another liquid suitable for consumption by an animal and oral administration is by the animal consuming the food, water, and/or other liquid suitable for consumption by an animal with the added composition including the microorganisms. In some embodiments, a solid powder, beads, and/or micro-particles including, but not limited to including the microorganisms, are combined with fiber and/or one or more excipients before being added to food, water, and/or another liquid suitable for consumption by an animal.

In some embodiments, the composition including, but not limited to including the microorganisms, is administered rectally. In some embodiments, tablets and/or capsules including the microorganisms, as described above, are administered rectally. In some embodiments, a solid powder, beads, and/or micro-particles including, but not limited to including the microorganisms, are administered rectally. In some embodiments, a solid powder, beads, and/or micro-particles including, but not limited to including the microorganisms, are added to, and/or combined with, a liquid or semi-solid suitable for rectal administration prior to the rectal administration.

As a non-limiting example of a composition and the processing thereof, a sample of fecal material from a donor may be cleaned of outside contamination. After cleaning, the sample or a portion of the sample is weighed, and a cryoprotectant, such as glycerol (vegetable glycerol), may be added, for example, at a minimum of 20% by weight (5 parts by weight fecal material to 1 part or more by weight cryoprotectant). The fecal material may be mixed with the cryoprotectant, and then flattened on parchment paper before freeze drying. After the freeze drying, the freeze-dried material may be subjected to size reduction by grinding with a mortar and pestle, using a coffee grinder, or a combination thereof. The resulting powder may be filled into capsules, and then the capsules may be enteric coated with a coating such as food grade shellac. The size of capsule used will depend upon the species of non-human animal donor and recipient. As non-limiting examples, a size 4 capsule may be used for cats and very small dogs and a size 0 capsule for dogs.

### Methods of Therapeutic Administration

Embodiments of the present invention encompass methods of administration of compositions including microorganisms to a non-human animal suffering from a disease or condition. Embodiments of the invention encompass administration of any of the above-described compositions, but are not limited to the identified compositions. Embodiments of the present invention encompass methods of administration of solid oral dosage forms (solid composition for oral administration) including microorganisms to a non-human animal suffering from a disease or condition.

In some embodiments, the disease or condition is a gastro-intestinal disease, such as, but not limited to, one or more of the following: colitis, constipation, acute or chronic diarrhea, gastritis, gastroenteritis, including hemorrhagic gastroenteritis, irritable or inflammatory bowel disease, irritable bowel syndrome, pancreatitis, small intestinal malabsorption, vomiting and regurgitation. Conditions may be caused by a variety of factors, including parasites, such as Tritrichomonas foetus; infectious bacteria, such as Campylobacter. Clostridium perfringens, and C. difficile; and viruses, such as Parvovirus.

In some embodiments, the disease or condition treated includes, but is not limited to including, one or more of the following: dermatitis, including atopic dermatitis—other skin conditions, diabetes, and kidney disease. Embodiments of the present invention encompass methods of administration of a composition, such as but not limited to a solid oral dosage forms (solid composition for oral administration), including microorganisms to a non-human animal suffering from an intolerance to food, including for example a food allergy, food sensitivity or a reaction to food. Non-limiting examples of food allergies include lactose intolerance. In some embodiments, the non-human animal is suffering from more than one disease or condition, and/or intolerance to food (a specific condition).

In some embodiments, the treatment regimen is a low dose approach designed to minimize stress on the patient.

In some embodiments, the treatment regimen for administration of a solid oral dosage is one to three capsules per day with food for a time period ranging from 1 to 60 days. In some embodiments, the period of administration is from 7 to 30 days. In preferred embodiments, the time period of administration is 25 days±5 days. The size of the capsule used (or size of tablet used for tablet dosage forms) will depend upon the size of the recipient and/or the typical size of animals in the same treatment group as the recipient. As non-limiting examples, a size 4 capsule (0.2 ml volume) may be used for cats and very small dogs, and a size 0 (0.68 ml volume) capsule for medium to large sized dogs. In some embodiments, a size 4 capsule (0.2 ml volume) includes 0.16 (±0.02) grams of powder, the powder being a freeze-dried, lyophilized, and/or spray-dried powder of a mixture comprising fecal material and an excipient with the fecal material being 20 wt % to 80 wt % of the powder. In some embodiments, a size 0 capsule (0.68 ml volume) includes 0.4 (±0.04) grams of powder, the powder being a freeze-dried, lyophilized, and/or spray-dried powder of a mixture comprising fecal material and an excipient with the fecal material being 20 wt % to 80 wt % of the powder. In some embodiments, each capsule contains from about 10³ CFU/ml to about 10¹¹ CFU/ml. In some embodiments, each capsule contains from about 10³ CFU/ml to about 10⁶ CFU/ml. In some embodiments, each capsule contains from about 10⁵ CFU/ml to about 10¹¹ CFU/ml. In some embodiments, each capsule contains from about 10⁸ CFU/ml to about 10¹¹ CFU/ml. In some embodiments, each capsule contains from about 10⁶ CFU/ml to about 10⁸ CFU/ml. In some embodiments, each dose contains from about 10³ CFU/ml to about 10¹¹ CFU/ml. In some embodiments, each dose contains from about 10³ CFU/ml to about 10⁶ CFU/ml. In some embodiments, each dose contains from about 10⁵ CFU/ml to about 10¹¹ CFU/ml. In some embodiments, each dose contains from about 10⁸ CFU/ml to about 10¹¹ CFU/ml. In some embodiments, each dose contains from about 10⁶ CFU/ml to about 10⁸ CFU/ml.

In some embodiments, the administration is of a dose of 1.7×10 CFU/Kg to 8.9×10⁹ CFU/Kg for a cat. In some embodiments, the administration is of a dose of 1.2×10 CFU/Kg to 1.5×10¹⁰ CFR/kg for a dog.

Administration with food (administration concurrent with consumption of food) may be administration of the composition within 15 minutes of consuming a meal, for example, up to 10 minutes before or after consuming a meal.

In some embodiments, methods of administration of compositions including microorganisms are concurrent with administration of fiber to a non-human animal suffering from a disease or condition. In some embodiments, concurrent is within 10 minutes. The fiber administration may be by administration of a composition including microorganisms and fiber, co-administration of fiber, or administration with food high in fiber, or a combination thereof. In some embodiments, the fiber is administered in an amount of 0.01 to 10 g/kg, preferably 0.01 to 0.5 g/kg, more preferably from 0.01 to 10 mg/kg. In some embodiments, the fiber administered is in the amount of 0.05 to 5 mg/kg. mg/kg. In some embodiments, fiber is co-administered by administration of one or more capsules, either enteric coated or not enteric coated, at the same time (within 5 minutes) of administration of a dosage form, such as but not limited to, a capsule including a composition including microorganisms as described herein. In some embodiments, administration of a composition including microorganisms is with food, the food being high in fiber. For a dog, a “high fiber” food is more than 5% by weight fiber, preferably, more than 6% by weight fiber, and more preferably, more than 7% by weight fiber. For a cat, a “high fiber” food is more than 3.5% by weight fiber, preferably, more than 4.0% by weight fiber, and more preferably, more than 4.5% by weight fiber.

The recipients, who may be patients, receiving a microbial therapy are generally taxonomically similar to the donor if any part of the microorganisms in the composition is obtained from a donor, for example from the same genus, the same species, or the same breed. In some embodiments, the recipient may be taxonomically different. As an example, a horse may be the recipient and the donor may be a donkey or a mule. As noted above, in preferred embodiments, the recipients, who may be patients, are non-human mammals, including both farm animals and companion animals. Examples of farm animals include, but are not limited to including, cows, horses, donkeys, mules, pigs, sheep, and goats. Examples of companion mammals include, but are not limited to including, cats, dogs, ferrets, mice, gerbils, rats, hamsters, and guinea pigs. In some embodiments, the patients are only companion mammals. In some embodiments, the patients are cats, dogs, and ferrets.

Embodiments of the invention encompass administration of the compositions where the compositions are intended for administration to feline patients (or subjects), and the compositions include, but are not limited to including, microorganisms, and the administration results in a significant change in at least one of the following microorganisms of the microbiota of the patient: Blautia, Oscillospira, Ruminococcus, Lachnospiraceae g1, Clostridiales f1. Embodiments of the invention encompass administration of the compositions where the compositions are intended for administration to canine patients (or subjects), and the compositions include, but are not limited to including, microorganisms, and the administration results in a significant change in at least one of the following microorganisms of the microbiota of the patient: Bacteroides, Enterococcus, Streptococcus, Ruminococcus, Blautia, Dorea, Prevotella, Clostridiaceae g1, Lachnospiraceae g1, Coprococcus, Oscillospira, Eubacterium, Clostridiales f1.

Non-limiting embodiments of the invention are described in the following number paragraphs:

Paragraph [0001]: Embodiments of the present invention encompass solid compositions for oral administration comprising microorganisms, the microorganisms comprising a combination of bacterial strains comprising members of the following groups: Blautia, a member of Clostridiales, a member of Ruminococcaceae, Eubacterium, Faecalibacterium, Oscillospira, Phascolarctobacterium, a member of Mogibacteriaceae, a member of Succinivibrionaceae, Coprococcus, Roseburia, and Catenibacterium.
Paragraph [0002] Embodiments of the present invention encompass solid compositions for oral administration comprising microorganisms, the microorganisms comprising a combination of bacterial taxa comprising members of the following groups: Blautia, a member of Clostridiales, a member of Ruminococcaceae, Eubacterium, Faecalibacterium, Oscillospira, Phascolarctobacterium, a member of Mogibacteriaceae, a member of Succinivibrionaceae, Coprococcus, Roseburia, and Catenibacterium.
Paragraph [0003]: In some embodiments of the present invention, such as, but not limited to, those described in paragraphs [0001] and [0002], the composition comprises a fecal material sample from a non-human mammal.
Paragraph [0004]: In some embodiments of the present invention, such as, but not limited to, those described in paragraphs [0001] and [0002], the composition comprises at least a portion of a fecal material sample from a non-human mammal.
Paragraph [0005]: In some embodiments of the present invention, such as, but not limited to, those described in in paragraphs [0001] and [0002], the microorganisms of the composition comprises a fecal microbiota of a non-human mammal or substantially a fecal microbiota of a non-human mammal.
Paragraph [0006]: In some embodiments of the present invention, such as, but not limited to, those described in paragraphs [0001] and [0002], the microorganisms of the composition are a fecal microbiota of a non-human mammal or substantially a fecal microbiota of a non-human mammal.
Paragraph [0007]: In some embodiments of the present invention, such as, but not limited to, those described in paragraphs [0003]-[0006], the fecal sample is a feline fecal sample.
Paragraph [0008]: In some embodiments of the present invention, such as, but not limited to, those described in paragraphs [0003]-[0006], the fecal sample is a canine fecal sample.
Paragraph [0009]: In some embodiments of the present invention, such as, but not limited to, those described in paragraphs [0003]-[0006], the fecal sample is a fecal sample from a ferret.
Paragraph [0010]: In some embodiments of the present invention, such as, but not limited to, those described in paragraphs [0003]-[0006], the fecal sample is a fecal sample from a rabbit.
Paragraph [0011] In some embodiments of the present invention, such as, but not limited to, those described in paragraphs [0003]-[0006], the fecal sample is a fecal sample from a horse.
Paragraph [0012] In some embodiments of the present invention, such as, but not limited to, those described in paragraphs [0001] and [0002], the composition comprises one or more cultured microbial taxa and/orstrains.
Paragraph [0013] In some embodiments of the present invention, such as, but not limited to, those described in paragraphs [0001] and [0002], the composition comprises one or more isolated microbial strains.
Paragraph [0014] In some embodiments of the present invention, such as, but not limited to, those described in paragraphs [0001] and [0002], the composition comprises one or more isolated microbial taxa.
Paragraph [0015] In some embodiments of the present invention, such as, but not limited to, those described in paragraphs [0001] and [0002], the composition comprises one or more enhanced microbial taxa and/or strains.
Paragraph [0016] In some embodiments of the present invention, such as, but not limited to, those described in paragraphs [0001]-[0011], the microorganisms of the composition are a combination of a fecal microbiota of a non-human mammal or substantially a fecal microbiota of a non-human mammal, and one or more microbial strains, the microbial strains being cultured, isolated, enhanced, or a combination thereof.
Paragraph [0017] In some embodiments of the present invention, such as, but not limited to, those described in paragraphs [0001]-[0016], each capsule contains from about 10³ CFU/ml to about 10¹¹ CFU/ml.
Paragraph [0018] In some embodiments of the present invention, such as, but not limited to, those described in paragraphs [0001]-[0017], the composition comprises an excipient.
Paragraph [0019] In some embodiments of the present invention, such as, but not limited to, those described in paragraph [0018], the excipient is selected from the group consisting of glycol, glycerol, vegetable glycerol, dimethylsulfoxide, 1,3-propanediol, ethanol, polyvinyl alcohol, propylene glycol, trimethylene glycol, diethylene glycol, polyethylene glycol, polypropylene glycol, polyethylene oxide, mannitol, D-Mannitol, D-Sorbitol, sorbitol, dulcitol, xylitol, glucose, D-Glucose, xylose, sucrose, lactose, maltose, trehalose, raffinose, dextran, mannan, dextrin, gum arabic (acacia), acetamide, methylacetamide, dimethylformamide, dimethyl-acetamide, succinimide, methylpyrrolidone, polyvinylpyrrolidone, proline, glycine, glutamic acid, aminobutyric acid, glutaric acid, ammonium acetate, cysteines, EDTA, blood serum, fetal calf serum, albumins, bovine serum albumin (BSA), gelatin, casein hydrolysate, starch hydolysate, hydroxypropyl cellulose, methylcellulose, ethylcellulose, hydroxypropyl methylcellulose, peptones, shell extract, glycoproteins, mucin, valinomycin, gramicidin, yeast extract, malt extract, skim milk, dairy milk, soy milk, fish oil, honey, polysorbate 80, polysorbate 20, polysorbate 60, polyethylene glycol p-(1,1,3,3-tetramethylbutyl)-phenyl ether, macrocyclon, glycine betaine, and combinations thereof.
Paragraph [0020] In some embodiments of the present invention, such as, but not limited to, those described in paragraphs [0001]-[0019], the microorganisms are frozen, freeze-dried, lyophilized, spray-dried, or a combination thereof.
Paragraph [0021] In some embodiments of the present invention, such as, but not limited to, those described in paragraphs [0001]-[0020], the composition is in the form of a powder, particles, granules, hard-shell capsule, tablet, or combination thereof.
Paragraph [0022] In some embodiments of the present invention, such as, but not limited to, those described in paragraphs [0001]-[0021], the composition is a capsule comprising the microorganisms, the microorganisms being frozen, freeze-dried, lyophilized, spray-dried, or a combination thereof.
Paragraph [0023] In some embodiments of the present invention, such as, but not limited to, those described in paragraph [0022], the capsules are coated.
Paragraph [0024] In some embodiments of the present invention, such as, but not limited to, those described in paragraph [0023], the capsule coating is an enteric coating.
Paragraph [0025] In some embodiments of the present invention, such as, but not limited to, those described in paragraph [0024], the enteric coating comprises food-grade shellac.
Paragraph [0026] Embodiments of the present invention encompass methods of preparing an oral composition for treating a gastrointestinal disease or condition in a non-human animal, the method comprising:

(a) Obtaining samples of fecal material from one or more individual non-human mammal donors, the donors being of the same species;

(b) Screening the fecal material samples for at least one pathogen;

(c) Eliminating the fecal material sample(s) with pathogens, if any;

(d) Screening the fecal material samples remaining after pathogen screening for bacterial diversity;

(e) Eliminating the fecal material sample(s) that do not meet the following criteria, if any: The presence of specific pathogens; poor fecal consistency.

If criteria are met, processing the fecal material sample to form a composition for oral administration to a non-human mammal.

Paragraph [0027] In some embodiments of the present invention, such as, but not limited to, those described in paragraph [0026], donors are screened for health, and only healthy donors are included in step (a).
Paragraph [0028] In some embodiments of the present invention, such as, but not limited to, those described in paragraphs [0026] and [0027], if all fecal samples are eliminated after step (e), repeating steps (a)-(e) on one or more occasions until at least one fecal material sample is not eliminated after step (e).
Paragraph [0029] In some embodiments of the present invention, such as, but not limited to, those described in paragraphs [0026]-[0028], the non-human mammal donors are cats; and the pathogens screened for include Clostridium difficile toxins A and B, Cryptosporidium spp, Salmonella spp, feline coronavirus, feline parvovirus (Panleukopenia), Giardia spp, canine parvovirus 2, and Tritrichomonas foetus.
Paragraph [0030] In some embodiments of the present invention, such as, but not limited to, those described in paragraphs [0026]-[0028], the non-human mammal donors are dogs; and the pathogens screened for include Clostridium difficile toxins A and B, Cryptosporidium spp, Salmonella spp, Giardia spp, Salmonella spp, Giardia spp, canine parvovirus 2, Clostridium perfringens antigen, alpha toxin, and beta toxin.
Paragraph [0031] In some embodiments of the present invention, such as, but not limited to, those described in paragraphs [0026]-[0028], the non-human mammal donors are ferrets; and the pathogens screened for include Cryptosporidium, Giardia spp., Canine distemper virus, Lawsonia, Campylobacter jejuni, Ferret coronavirus, Helicobacter spp, Salmonella spp.
Paragraph [0032] In some embodiments of the present invention, such as, but not limited to, those described in paragraphs [0026]-[0031], the composition is a liquid composition.
Paragraph [0033] In some embodiments of the present invention, such as, but not limited to, those described in paragraphs [0026]-[0031], the composition is a solid composition.
Paragraph [0034] In some embodiments of the present invention, such as, but not limited to, those described in paragraph [0033], the composition is in the form of a powder, particles, granules, capsule, tablet, or a combination thereof.
Paragraph [0035] In some embodiments of the present invention, such as, but not limited to, those described in paragraphs [0026]-[0034], the composition comprises an excipient.
Paragraph [0036] In some embodiments of the present invention, such as, but not limited to, those described in paragraph [0035], wherein processing the fecal samples comprises removing any outside contamination, optionally adding an excipient to the fecal sample, and subsequently freezing, freeze-drying, spray-drying, lyophilizing, or a combination thereof, the fecal sample and optional excipient to form a solid.
Paragraph [0037] In some embodiments of the present invention, such as, but not limited to, those described in paragraph [0036], wherein the processing further comprises reducing the size of at least a portion of the solid to form a powder, and subsequently at least partially filling one or more capsules with at least a portion of the powder.
Paragraph [0038] In some embodiments of the present invention, such as, but not limited to, those described in paragraph [0037], wherein the processing further comprises coating at least some of the capsules.
Paragraph [0039] In some embodiments of the present invention, such as, but not limited to, those described in paragraph [0038], wherein at least one coating on the capsules is an enteric coating.
Paragraph [0040] Embodiments of the present invention encompass methods of treating gastrointestinal disease or condition in a non-human mammal comprising: administering to a non-human mammal a composition comprising microorganisms, the microorganisms comprising a combination of bacterial taxa comprising members of the following groups: Blautia, a member of Clostridiales, a member of Ruminococcaceae, Eubacterium, Faecalibacterium, Oscillospira, Phascolarctobacterium, a member of Mogibacteriaceae, a member of Succinivibrionaceae, Coprococcus, Roseburia, and Catenibacterium.
Paragraph [0041] Embodiments of the present invention encompass methods of treating gastrointestinal disease or condition in a non-human mammal comprising: administering to a non-human mammal a composition comprising microorganisms, the microorganisms comprising a combination of bacterial strains comprising members of the following groups: Blautia, a member of Clostridiales, a member of Ruminococcaceae, Eubacterium, Faecalibacterium, Oscillospira, Phascolarctobacterium, a member of Mogibacteriaceae, a member of Succinivibrionaceae, Coprococcus, Roseburia, and Catenibacterium.
Paragraph [0042] In some embodiments of the present invention, such as, but not limited to, those described in paragraph [0040] and [0041], the composition is a composition for oral administration.
Paragraph [0043] In some embodiments of the present invention, such as, but not limited to, those described in paragraph [0042], the composition is a solid composition.
Paragraph [0044] In some embodiments of the present invention, such as, but not limited to, those described in paragraphs [0040]-[0043], the non-human mammal is a cat.
Paragraph [0045] In some embodiments of the present invention, such as, but not limited to, those described in paragraph [0044], the gastrointestinal disease is colitis, constipation, acute or chronic diarrhea, gastritis, gastroenteritis, inflammatory bowel disease, irritable bowel disease, irritable bowel syndrome, pancreatitis, small intestinal malabsorption, vomiting and regurgitation.
Paragraph [0046] In some embodiments of the present invention, such as, but not limited to, those described in paragraphs [0040]-[0043], the non-human mammal is a dog.
Paragraph [0047] In some embodiments of the present invention, such as, but not limited to, those described in paragraph [0046], the gastrointestinal disease is colitis, constipation, acute or chronic diarrhea, gastritis, gastroenteritis, inflammatory bowel disease, irritable bowel disease, irritable bowel syndrome, pancreatitis, small intestinal malabsorption, vomiting and regurgitation.
Paragraph [0048] In some embodiments of the present invention, such as, but not limited to, those described in paragraphs [0040]-[0043], the non-human mammal is a ferret.
Paragraph [0049] In some embodiments of the present invention, such as, but not limited to, those described in paragraph [0048], the gastrointestinal disease is colitis, constipation, acute or chronic diarrhea, gastritis, gastroenteritis, inflammatory bowel disease, irritable bowel disease, irritable bowel syndrome, pancreatitis, small intestinal malabsorption, vomiting and regurgitation.
Paragraph [0050] In some embodiments of the present invention, such as, but not limited to, those described in paragraphs [0040]-[0049], the administration comprises administration of a dose of one capsule one, two, or three times daily with food for a period of 4 days to 30 days.
Paragraph [0051] In some embodiments of the present invention, such as, but not limited to, those described in paragraph [0050], the time period of administration is 10 days to 30 days.
Paragraph [0052] In some embodiments of the present invention, such as, but not limited to, those described in paragraph [0051], the time period of administration is 22 days to 28 days.
Paragraph [0053] Embodiments of the present invention encompass solid compositions for oral administration comprising fecal material from a non-human animal.
Paragraph [0054] In some embodiments of the present invention, such as, but not limited to, those described in paragraph [0053], the fecal material is from only one individual non-human mammal.
Paragraph [0055] In some embodiments of the present invention, such as, but not limited to, those described in paragraphs [0053] and [0054], the composition comprises fiber.
Paragraph [0056] In some embodiments of the present invention, such as, but not limited to, those described in paragraphs [0053] and [0054], the fecal material is feline fecal material.
Paragraph [0057] In some embodiments of the present invention, such as, but not limited to, those described in paragraphs [0053] and [0054], the fecal material is canine fecal material.
Paragraph [0058] In some embodiments of the present invention, such as, but not limited to, those described in paragraphs [0053] and [0054], the fecal material is fecal material from a ferret.
Paragraph [0059] In some embodiments of the present invention, such as, but not limited to, those described in paragraphs [0053] and [0054], the fecal material is fecal material from a rabbit.
Paragraph [0060] In some embodiments of the present invention, such as, but not limited to, those described in paragraphs [0053] and [0054], the fecal material is fecal material from a horse, goat, donkey, cow, pig, or mule.
Paragraph [0061] In some embodiments of the present invention, such as, but not limited to, those described in paragraphs [0053]-[0060], the composition comprises one or more cultured microbial taxa.
Paragraph [0062] In some embodiments of the present invention, such as, but not limited to, those described in paragraphs [0053]-[0061], the composition comprises one or more isolated microbial taxa.
Paragraph [0063] In some embodiments of the present invention, such as, but not limited to, those described in paragraphs [0053]-[0062], the composition comprises one or more enhanced microbial taxa.
Paragraph [0064] In some embodiments of the present invention, such as, but not limited to, those described in paragraphs [0053]-[0063], the composition comprises one or more microbial taxa obtained by fermentation.
Paragraph [0065] In some embodiments of the present invention, such as, but not limited to, those described in paragraph [0064], the one or more microbial taxa obtained by fermentation are obtained by fermentation of freeze-dried fecal material of a non-human animal.
Paragraph [0066] In some embodiments of the present invention, such as, but not limited to, those described in paragraphs [0064] and [0065], the at least one of the microbial taxa obtained by fermentation is one of the following: Dorea, Ruminoccoccus, a member of the family Lachnospiraceae that is not Blautia and is not Dorea, Collinsella, Blautia, a member of the order Clostridiales, a member of the family Ruminococcaceae, Clostridium, a member of the order Clostridiales, or Sutterella.
Paragraph [0067] In some embodiments of the present invention, such as, but not limited to, those described in paragraphs [0064] and [0065], the at least one of the microbial taxa obtained by fermentation is one of the following: Ruminoccoccus, Blautia, Dorea, a member of the family Clostridiaceae, a member of the family Lachnospiraceae that is not Blautia and is not Dorea, a member of the order Clostridiales, Sutterella, Eubacterium, Collinsella, or a member of the family Erysipelotrichaceae.
Paragraph [0068] In some embodiments of the present invention, such as, but not limited to, those described in paragraphs [0053]-[0067], the composition comprises microorganisms, the microorganisms comprising a combination of a fecal microbiota of a non-human mammal or substantially a fecal microbiota of a non-human mammal, and one or more microbial taxa, the microbial taxa being cultured, isolated, enhanced, product of fermentation, or a combination thereof.
Paragraph [0069] In some embodiments of the present invention, such as, but not limited to, those described in paragraphs [0053]-[0068], the composition comprises an excipient.
Paragraph [0070] In some embodiments of the present invention, such as, but not limited to, those described in paragraphs [0053]-[0069], the fecal material is frozen, freeze-dried, lyophilized, spray-dried, or a combination thereof, and optional additional microorganisms, if present, are frozen, freeze-dried, lyophilized, spray-dried, or a combination thereof.
Paragraph [0071] In some embodiments of the present invention, such as, but not limited to, those described in paragraphs [0053]-[0070], the composition is in the form of a powder, particles, granules, hard-shell capsule, tablet, or combination thereof.
Paragraph [0072] In some embodiments of the present invention, such as, but not limited to, those described in paragraph [0071], the composition is a capsule or tablet, and each capsule or tablet comprises from about 10³ CFU/ml to about 10¹¹ CFU/ml.
Paragraph [0073] In some embodiments of the present invention, such as, but not limited to, those described in paragraph [0071], the composition is a capsule or tablet, and each capsule or tablet comprises from about 10⁵ CFU/ml to about 10¹¹ CFU/ml.
Paragraph [0074] In some embodiments of the present invention, such as, but not limited to, those described in paragraph [0071], the composition is a capsule or tablet, and each capsule or tablet comprises from about 10⁶ CFU/ml to about 10¹⁰ CFU/ml.
Paragraph [0075] In some embodiments of the present invention, such as, but not limited to, those described in paragraphs [0053]-[0074], the composition is a capsule comprising the fecal material, the fecal material being frozen, freeze-dried, lyophilized, spray-dried, or a combination thereof, and optionally comprising additional microorganisms, and the additional microorganisms, if present, being frozen, freeze-dried, lyophilized, spray-dried, or a combination thereof.
Paragraph [0076] In some embodiments of the present invention, such as, but not limited to, those described in paragraph [0075], one or more excipients are present and at least one of the one or more excipients is glycol, glycerol, vegetable glycerol, dimethylsulfoxide, 1,3-propanediol, ethanol, polyvinyl alcohol, propylene glycol, trimethylene glycol, diethylene glycol, polyethylene glycol, polypropylene glycol, polyethylene oxide, mannitol, D-Mannitol, D-Sorbitol, sorbitol, dulcitol, xylitol, glucose, D-Glucose, xylose, sucrose, lactose, maltose, trehalose, raffinose, dextran, mannan, dextrin, gum arabic (acacia), acetamide, methylacetamide, dimethylformamide, dimethyl-acetamide, succinimide, methylpyrrolidone, polyvinylpyrrolidone, proline, glycine, glutamic acid, aminobutyric acid, glutaric acid, ammonium acetate, one or more cysteines, EDTA, blood serum, fetal calf serum, one or more albumins, bovine serum albumin (BSA), gelatin, casein hydrolysate, starch hydolysate, hydroxypropyl cellulose, methylcellulose, ethylcellulose, hydroxypropyl methylcellulose, one or more peptones, shell extract, one or more glycoproteins, mucin, valinomycin, gramicidin, yeast extract, malt extract, skim milk, dairy milk, soy milk, fish oil, honey, polysorbate 80, polysorbate 20, polysorbate 60, polyethylene glycol p-(1,1,3,3-tetramethylbutyl)-phenyl ether, macrocyclon, glycine betaine, or a combination thereof.
Paragraph [0077] In some embodiments of the present invention, such as, but not limited to, those described in paragraphs [0075] and [0076], the capsules are coated.
Paragraph [0078] In some embodiments of the present invention, such as, but not limited to, those described in paragraph [0077], the capsule coating comprises an enteric coating.
Paragraph [0079] In some embodiments of the present invention, such as, but not limited to, those described in paragraph [0078], the enteric coating comprises food-grade shellac.
Paragraph [0080] Embodiments of the present invention encompass solid compositions for administration comprising microorganisms, the microorganisms comprising:
(a) microorganisms of at least one of the following taxa:
Ruminoccoccus, at least one member of the family Clostridiaceae, at least one member of the family Lachnospiraceae that is not Blautia and is not Dorea, Blautia, Fusobacterium, Dorea, at least one member of the order Clostridiales, Bacteroides, Sutterella, Eubacterium, Collinsella, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium; and
(b) microorganisms of at least one of the following taxa, which may be the same as or different from the microorganisms of the at least one taxa in (a): at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, Megamonas;
and/or
   microorganisms of at least one of the following taxa, which may be the same as or different from the microorganisms of the at least one taxa in (a): Dorea, at least one member of the order Clostridiales, Sutterella, Eubacterium, Collinsella, at least one member of the family Erysipelotrichaceae, Megamonas, at least one member of the family Ruminococcaceae, Clostridium, at least another member of the family Clostridiaceae, at least another member of the family Lachnospiraceae that is not Blautia and that is not Dorea, Faecalibacterium.
      Paragraph [0081] In some embodiments of the present invention, such as, but not limited to, those described in paragraph [0080], the compositions are for enteral administration.
      Paragraph [0082] In some embodiments of the present invention, such as, but not limited to, those described in paragraphs [0080] and [0081], the compositions are for oral administration.
      Paragraph [0083] In some embodiments of the present invention, such as, but not limited to, those described in paragraphs [0080]-[0082], wherein the composition comprises at least two of the taxa in (a).
      Paragraph [0084] In some embodiments of the present invention, such as, but not limited to, those described in paragraphs [0080]-[0083], wherein the composition comprises at least three of the taxa in (a).
      Paragraph [0085] In some embodiments of the present invention, such as, but not limited to, those described in paragraphs [0080]-[0084], wherein the composition comprises at least four of the taxa in (a).
      Paragraph [0086] In some embodiments of the present invention, such as, but not limited to, those described in paragraphs [0080]-[0085], wherein the composition comprises at least five of the taxa in (a).
      Paragraph [0087] In some embodiments of the present invention, such as, but not limited to, those described in paragraphs [0080]-[0086], wherein the composition comprises at least six of the taxa in (a).
      Paragraph [0088] In some embodiments of the present invention, such as, but not limited to, those described in paragraphs [0080]-[0087], the composition comprises fiber.
      Paragraph [0089] In some embodiments of the present invention, such as, but not limited to, those described in paragraph [0088], the fiber comprises one or more oligosaccharides, resistant starch, pectin, one or more beta-glucans, one or more xylooligosaccharides, or a combination thereof.
      Paragraph [0090] In some embodiments of the present invention, such as, but not limited to, those described in paragraph [0089], the fiber comprises one or more oligosaccharides, at least one being a fructan, at least one being a galactan, or at least one being a fructan and at least one being a galactan.
      Paragraph [0091] In some embodiments of the present invention, such as, but not limited to, those described in paragraphs [0080]-[0090], the composition comprises fecal material from a non-human mammal.
      Paragraph [0092] In some embodiments of the present invention, such as, but not limited to, those described in paragraph [0091], the fecal material is from only one individual non-human mammal.
      Paragraph [0093] In some embodiments of the present invention, such as, but not limited to, those described in paragraphs [0091] and [0092], the fecal material is feline fecal material.
      Paragraph [0094] In some embodiments of the present invention, such as, but not limited to, those described in paragraph [0093], the composition comprises microorganisms of at least two of the following taxa: Dorea, Ruminoccoccus, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella, Prevotella, Eubacterium, Oscillospira, at least another member of the family Clostridiaceae, at least another member of the family Lachnospiraceae that is not Blautia and that is not Dorea, Ruminococcus, Coprococcus, Parabacteroides, Fusobacterium, Faecalibacterium, Megamonas.
      Paragraph [0095] In some embodiments of the present invention, such as, but not limited to, those described in paragraph [0093], the composition comprises microorganisms of at least three of the following taxa: Dorea, Ruminoccoccus, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella, Prevotella, Eubacterium, Oscillospira, at least another member of the family Clostridiaceae, at least another member of the family Lachnospiraceae that is not Blautia and that is not Dorea, Ruminococcus, Coprococcus, Parabacteroides, Fusobacterium, Faecalibacterium, Megamonas.
      Paragraph [0096] In some embodiments of the present invention, such as, but not limited to, those described in paragraph [0093], the composition comprises microorganisms of at least five of the following taxa: Dorea, Ruminoccoccus, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella, Prevotella, Eubacterium, Oscillospira, at least another member of the family Clostridiaceae, at least another member of the family Lachnospiraceae that is not Blautia and that is not Dorea, Ruminococcus, Coprococcus, Parabacteroides, Fusobacterium, Faecalibacterium, Megamonas.
      Paragraph [0097] In some embodiments of the present invention, such as, but not limited to, those described in paragraph [0093], the composition comprises microorganisms of at least one of the following taxa: Dorea, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella.
      Paragraph [0098] In some embodiments of the present invention, such as, but not limited to, those described in paragraph [0093], the composition comprises microorganisms of at least two of the following taxa: at least one member of the order Clostridiales, at least one member of the family Ruminococcaceae, Clostridium, at least one member of the family Clostridiaceae, Sutterella.
      Paragraph [0099] In some embodiments of the present invention, such as, but not limited to, those described in paragraphs [0091] and [0092], the fecal material is canine fecal material.
      Paragraph [0100] In some embodiments of the present invention, such as, but not limited to, those described in paragraph [0099], the composition comprises microorganisms of at least two of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, at least one member of the family Erysipelotrichaceae, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium.
      Paragraph [0101] In some embodiments of the present invention, such as, but not limited to, those described in paragraph [0099], the composition comprises microorganisms of at least three of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, at least one member of the family Erysipelotrichaceae, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium.
      Paragraph [0102] In some embodiments of the present invention, such as, but not limited to, those described in paragraph [0099], the composition comprises microorganisms of at least five of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, at least one member of the family Erysipelotrichaceae, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium.
      Paragraph [0103] In some embodiments of the present invention, such as, but not limited to, those described in paragraph [0099], the composition comprises at least one of the following taxa: Ruminoccoccus, at least one member of the family Clostridiaceae, at least one member of the family Lachnospiraceae that is not Blautia and that is not Dorea, Blautia, Dorea, at least one member of the order Clostridiales, Sutterella, Eubacterium, Collinsella, at least one member of the family Erysipelotrichaceae.

Paragraph [0104] In some embodiments of the present invention, such as, but not limited to, those described in paragraph [0099], wherein the composition comprises at least two of the following taxa: Dorea, at least one member of the order Clostridiales, Sutterella, Eubacterium, Collinsella, at least one member of the family Erysipelotrichaceae.

Paragraph [0105] In some embodiments of the present invention, such as, but not limited to, those described in paragraphs [0091] and [0092], the fecal material is fecal material from a ferret.
Paragraph [0106] In some embodiments of the present invention, such as, but not limited to, those described in paragraphs [0091] and [0092], the fecal material is fecal material from a rabbit.
Paragraph [0107] In some embodiments of the present invention, such as, but not limited to, those described in paragraphs [0091] and [0092], the fecal material is fecal material from a horse.
Paragraph [0108] In some embodiments of the present invention, such as, but not limited to, those described in paragraphs [0080]-[0090], the microorganisms of the composition comprise a fecal microbiota of a non-human mammal.
Paragraph [0109] In some embodiments of the present invention, such as, but not limited to, those described in paragraphs [0080]-[0090], the microorganisms of the composition comprise substantially a fecal microbiota of a non-human mammal.
Paragraph [0110] In some embodiments of the present invention, such as, but not limited to, those described in paragraphs [0080]-[0109], the composition comprises one or more cultured microbial taxa and/or strains.
Paragraph [0111] In some embodiments of the present invention, such as, but not limited to, those described in paragraphs [0080]-[0110], the composition comprises one or more isolated microbial taxa and/or strains.
Paragraph [0112] In some embodiments of the present invention, such as, but not limited to, those described in paragraphs [0080]-[0111], the composition comprises one or more enhanced microbial taxa and/or strains.
Paragraph [0113] In some embodiments of the present invention, such as, but not limited to, those described in paragraphs [0080]-[0112], the composition comprises one or more microbial taxa and/or strains obtained by fermentation.

Paragraph [0014] In some embodiments of the present invention, such as, but not limited to, those described in paragraphs [0113], the microorganisms obtained by fermentation comprise microorganisms of: at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, and/or Megamonas.

Paragraph [0115] In some embodiments of the present invention, such as, but not limited to, those described in paragraphs [0113], the microorganisms obtained by fermentation comprise microorganisms of Dorea, at least one member of the order Clostridiales, Sutterella, Eubacterium, Collinsella, and/or at least one member of the family Erysipelotrichaceae.
Paragraph [0116] In some embodiments of the present invention, such as, but not limited to, those described in paragraphs [0080]-[0090], wherein the microorganisms of the composition are a combination of a fecal microbiota of a non-human mammal or substantially a fecal microbiota of a non-human mammal, and one or more microbial taxa, the microbial taxa being cultured, isolated, enhanced, obtained from fermentation, or a combination thereof.
Paragraph [011] In some embodiments of the present invention, such as, but not limited to, those described in paragraphs [0080]-[0116], the composition comprises an excipient.
Paragraph [0118] In some embodiments of the present invention, such as, but not limited to, those described in paragraphs [0080]-[0117], the microorganisms of the composition are frozen, freeze-dried, lyophilized, spray-dried, or a combination thereof.
Paragraph [0119] In some embodiments of the present invention, such as, but not limited to, those described in paragraphs [0080]-[0118], the composition is in the form of a powder, particles, granules, hard-shell capsule, tablet, or combination thereof.
Paragraph [0120] In some embodiments of the present invention, such as, but not limited to, those described in paragraph [0119], the composition is a capsule, and each capsule comprises from about 10³ CFU/ml to about 10¹¹CFU/ml.
Paragraph [0121] In some embodiments of the present invention, such as, but not limited to, those described in paragraph [0119], the composition is a capsule, and each capsule comprises from about 10⁵ CFU/ml to about 10¹¹CFU/ml.
Paragraph [0122] In some embodiments of the present invention, such as, but not limited to, those described in paragraph [0119], the composition is a capsule, and each capsule comprises from about 10⁶ CFU/ml to about 10¹⁰ CFU/ml.
Paragraph [0123] In some embodiments of the present invention, such as, but not limited to, those described in paragraph [0119], the composition is a tablet, and each tablet comprises from about 10³ CFU/ml to about 10¹¹ CFU/ml.
Paragraph [0124] In some embodiments of the present invention, such as, but not limited to, those described in paragraph [0119], the composition is a tablet, and each tablet comprises from about 10⁵ CFU/ml to about 10¹¹ CFU/ml.
Paragraph [0125] In some embodiments of the present invention, such as, but not limited to, those described in paragraph [0119], the composition is a tablet, and each tablet comprises from about 10⁶ CFU/ml to about 10¹⁰ CFU/ml.
Paragraph [0126] In some embodiments of the present invention, such as, but not limited to, those described in paragraphs [0119]-[0122], the composition comprises a capsule comprising the microorganisms, the microorganisms being frozen, freeze-dried, lyophilized, spray-dried, or a combination thereof.
Paragraph [0127] In some embodiments of the present invention, such as, but not limited to, those described in paragraph [0126], the composition comprises one or more excipients and at least one of the one or more excipients comprises glycol, glycerol, vegetable glycerol, dimethylsulfoxide, 1,3-propanediol, ethanol, polyvinyl alcohol, propylene glycol, trimethylene glycol, diethylene glycol, polyethylene glycol, polypropylene glycol, polyethylene oxide, mannitol, D-Mannitol, D-Sorbitol, sorbitol, dulcitol, xylitol, glucose, D-Glucose, xylose, sucrose, lactose, maltose, trehalose, raffinose, dextran, mannan, dextrin, gum arabic (acacia), acetamide, methylacetamide, dimethylformamide, dimethyl-acetamide, succinimide, methylpyrrolidone, polyvinylpyrrolidone, proline, glycine, glutamic acid, aminobutyric acid, glutaric acid, ammonium acetate, cysteines, EDTA, blood serum, fetal calf serum, albumins, bovine serum albumin (BSA), gelatin, casein hydrolysate, starch hydolysate, hydroxypropyl cellulose, methylcellulose, ethylcellulose, hydroxypropyl methylcellulose, peptones, shell extract, glycoproteins, mucin, valinomycin, gramicidin, yeast extract, malt extract, skim milk, dairy milk, soy milk, fish oil, honey, polysorbate 80, polysorbate 20, polysorbate 60, polyethylene glycol p-(1,1,3,3-tetramethylbutyl)-phenyl ether, macrocyclon, glycine betaine, or a combination thereof.
Paragraph [0128] In some embodiments of the present invention, such as, but not limited to, those described in paragraph [0127], the capsules are coated.
Paragraph [0129] In some embodiments of the present invention, such as, but not limited to, those described in paragraph [0128], the capsule coating comprises an enteric coating.
Paragraph [0130] In some embodiments of the present invention, such as, but not limited to, those described in paragraph [0129], the enteric coating comprises food-grade shellac.
Paragraph [0131] Embodiments of the present invention encompass methods of preparing a composition for treating a disease or condition in a non-human animal, the method comprising:

(a) Obtaining samples of fecal material from one or more individual non-human mammal donors, the donors being of the same species;

(b) Screening the fecal material samples for at least one pathogen;

(c) Eliminating the fecal material sample(s) with pathogens, if any;

(d) Screening the fecal material samples remaining after pathogen screening for bacterial diversity;

(e) Eliminating the fecal material sample(s) that do not meet the following criteria, if any:
The absence of specific pathogens; acceptable fecal consistency; the presence of microorganisms of at least one taxon of group (a), and/or at least one taxon of group (b):
   Group (a): the following taxa: at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, Megamonas;
   Group (b): the following taxa: Dorea, at least one member of the order Clostridiales, Sutterella, Eubacterium, Collinsella, at least one member of the family Erysipelotrichaceae, Megamonas, at least one member of the family Ruminococcaceae, Clostridium, at least another member of the family Clostridiaceae, at least another member of the family Lachnospiraceae that is not Blautia and that is not Dorea, Faecalibacterium.

(f) If criteria are met, processing at least a portion of at least one of the fecal material sample(s) to form a composition for oral administration to a non-human mammal.

Paragraph [0132] In some embodiments of the present invention, such as, but not limited to, those described in paragraph [0131], the compositions prepared by the method are for enteral administration.
Paragraph [0133] In some embodiments of the present invention, such as, but not limited to, those described in paragraph [0131] and [0132], the compositions prepared by the method are for oral administration.
Paragraph [0134] In some embodiments of the present invention, such as, but not limited to, those described in paragraph [0131]-[0133], donors are screened for health, and only healthy donors are included in step (a).
Paragraph [0135] In some embodiments of the present invention, such as, but not limited to, those described in paragraph [0131]-[0134], if all fecal samples are eliminated after step (e), repeating steps (a)-(e) on one or more occasions until at least one fecal material sample is not eliminated after step (e).
Paragraph [0136] In some embodiments of the present invention, such as, but not limited to, those described in paragraphs [0131]-[0135], the non-human mammal donors are cats;

and

wherein the pathogens screened for comprise Clostridium difficile toxins A and B, Cryptosporidium spp, Salmonella spp, feline coronavirus, feline parvovirus (Panleukopenia), Giardia spp, canine parvovirus 2, and Tritrichomonas foetus.

Paragraph [0137] In some embodiments of the present invention, such as, but not limited to, those described in paragraph [0136], in step (e) microorganisms of at least one of the taxa of group (a) are present.
Paragraph [0138] In some embodiments of the present invention, such as, but not limited to, those described in paragraph [0137], the microorganisms of the at least one of the taxa of group (a) are present at an abundance at least equal to the median abundance of healthy cats.
Paragraph [0139] In some embodiments of the present invention, such as, but not limited to, those described in paragraphs [0131]-[0135], the non-human mammal donors are dogs;

and

wherein the pathogens screened for include Clostridium difficile toxins A and B, Cryptosporidium spp, Salmonella spp, Giardia spp, canine parvovirus 2, Clostridium perfringens antigen, alpha toxin, and beta toxin.

Paragraph [0140] In some embodiments of the present invention, such as, but not limited to, those described in paragraph [0139], in step (e) microorganisms of at least one of the taxa of group (b) are present.
Paragraph [0141] In some embodiments of the present invention, such as, but not limited to, those described in paragraph [0140], the microorganisms of the at least one of the taxa of group (b) are present at an abundance at least equal to the median abundance of healthy dogs.
Paragraph [0142] In some embodiments of the present invention, such as, but not limited to, those described in paragraphs [0131]-[0135], the non-human mammal donors are ferrets;

and

wherein the pathogens screened for include Cryptosporidium, Giardia spp., Canine distemper virus, Lawsonia, Campylobacter jejuni, Ferret coronavirus, Helicobacter spp, Salmonella spp.

Paragraph [0143] In some embodiments of the present invention, such as, but not limited to, those described in paragraphs [0131]-[0142], the composition is a liquid composition.
Paragraph [0144] In some embodiments of the present invention, such as, but not limited to, those described in paragraphs [0131]-[0142], the composition is a solid composition.
Paragraph [0145] In some embodiments of the present invention, such as, but not limited to, those described in paragraph [0144], the composition is in the form of a powder, particles, granules, capsule, tablet, or a combination thereof.
Paragraph [0146] In some embodiments of the present invention, such as, but not limited to, those described in paragraphs [0144] and [0145], processing at least a portion of at least one of the fecal samples comprises removing any outside contamination, optionally adding one or more excipients to the fecal sample, and subsequently freezing, freeze-drying, spray-drying, lyophilizing, or a combination thereof, the fecal sample and optional excipient(s) to form a solid.
Paragraph [0147] In some embodiments of the present invention, such as, but not limited to, those described in paragraph [0146], the processing further comprises reducing the size of at least a portion of the solid to form a powder, and subsequently at least partially filling one or more capsules with at least a portion of the powder.
Paragraph [0148] In some embodiments of the present invention, such as, but not limited to, those described in paragraph [0147], the processing further comprises coating at least some of the capsules.
Paragraph [0149] In some embodiments of the present invention, such as, but not limited to, those described in paragraph [0148], at least one coating on the capsules is an enteric coating.
Paragraph [0150] In some embodiments of the present invention, such as, but not limited to, those described in paragraphs [0131]-[0149], the composition comprises an excipient. Paragraph [0151] In some embodiments of the present invention, such as, but not limited to, those described in paragraphs [0131]-[0150], the composition comprises fiber.
Paragraph [0152] In some embodiments of the present invention, such as, but not limited to, those described in paragraph [0151], the fiber comprises one or more oligosaccharides, resistant starch, pectin, one or more beta-glucans, one or more xylooligosaccharides, or a combination thereof.
Paragraph [0153] In some embodiments of the present invention, such as, but not limited to, those described in paragraph [0152], the fiber comprises one or more oligosaccharides, at least one being a fructan, at least one being a galactan, or at least one being a fructan and at least one being a galactan.
Paragraph [0154] Embodiments of the present invention encompass methods of treating a non-human mammal suffering from at least one disease or condition, the method comprising:

administering to a non-human mammal a composition comprising microorganisms, the microorganisms comprising:

(a) microorganisms of at least one of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium;

and

(b) microorganisms of at least one of the following taxa, which may be the same as or different from the microorganisms of the at least one taxa in (a): at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, Megamonas;

and/or

microorganisms of at least one of the following taxa, which may be the same as or different from the microorganisms of the at least one taxa in (a): Dorea, at least one member of the order Clostridiales, Sutterella, Eubacterium, Collinsella, at least one member of the family Erysipelotrichaceae, Megamonas, at least one member of the family Ruminococcaceae, Clostridium, at least another member of the family Clostridiaceae, at least another member of the family Lachnospiraceae that is not Blautia and that is not Dorea, Faecalibacterium.

Paragraph [0155] In some embodiments of the present invention, such as, but not limited to, those described in paragraph [0154], the composition is a composition for enteral administration.
Paragraph [0156] In some embodiments of the present invention, such as, but not limited to, those described in paragraphs [0154] and [0155], the composition is a composition for oral administration.
Paragraph [0157] In some embodiments of the present invention, such as, but not limited to, those described in paragraph [0154]-[0156], the composition is a solid composition.
Paragraph [0158] In some embodiments of the present invention, such as, but not limited to, those described in paragraphs [0154]-[0157], at least one disease or condition is a gastrointestinal disease or condition.
Paragraph [0159] In some embodiments of the present invention, such as, but not limited to, those described in paragraph [0158], the at least one gastrointestinal disease or condition comprises colitis, constipation, acute or chronic diarrhea, gastritis, gastroenteritis, irritable bowel syndrome, pancreatitis, small intestinal malabsorption, vomiting, regurgitation, hemorrhagic gastroenteritis, and/or inflammatory bowel disease.
Paragraph [0160] In some embodiments of the present invention, such as, but not limited to, those described in paragraphs [0154]-[0157], at least disease or condition comprises atopic dermatitis, dermatitis, one or more skin conditions, diabetes, kidney disease, or a combination thereof.
Paragraph [0161] In some embodiments of the present invention, such as, but not limited to, those described in paragraphs [0154]-[0157], at least disease or condition comprises atopic dermatitis, dermatitis, one or more skin conditions, diabetes, kidney disease, or a combination thereof.
Paragraph [0162] In some embodiments of the present invention, such as, but not limited to, those described in paragraphs [0154]-[0157], at least one disease or condition is infection with Tritrichomonas foetus, Campylobacter, Clostridium difficile, Clostridium perfringens, Parvovirus, or a combination thereof.
Paragraph [0163] In some embodiments of the present invention, such as, but not limited to, those described in paragraphs [0154]-[0157], at least disease or condition comprises a food allergy, food sensitivity, and/or a reaction to a food.
Paragraph [0164] In some embodiments of the present invention, such as, but not limited to, those described in paragraphs [0154]-[0163], the composition is a capsule and/or a tablet, and a unit dosage is one capsule or one tablet, and administration comprises administration of one, two, or three unit dosages one, two, or three times daily for a period of 4 days to 30 days.
Paragraph [0165] In some embodiments of the present invention, such as, but not limited to, those described in paragraph [0164], the time period of administration is 10 days to 30 days.
Paragraph [0166] In some embodiments of the present invention, such as, but not limited to, those described in paragraph [0165], the time period of administration is 22 days to 28 days.
Paragraph [0167] In some embodiments of the present invention, such as, but not limited to, those described in paragraphs [0164]-[0166], the administration of the unit dosage(s) is concurrent with consumption of food.
Paragraph [0168] In some embodiments of the present invention, such as, but not limited to, those described in paragraph [0167], the food is high fiber food.
Paragraph [0169] In some embodiments of the present invention, such as, but not limited to, those described in paragraphs [0167] and [0168], fiber is added to the food.
Paragraph [0170] In some embodiments of the present invention, such as, but not limited to, those described in paragraphs [0154]-[0169], the administration comprises concurrent administration of fiber, which may be in addition to or instead of the optional addition of fiber to food if the administration of the composition is concurrent with consumption of food.
Paragraph [0171] In some embodiments of the present invention, such as, but not limited to, those described in paragraph [0170], concurrent administration of fiber comprises administration of one or more capsules comprising fiber, one or more tablets comprising fiber, or both one or more capsules comprising fiber and one or more tablets comprising fiber.
Paragraph [0172] In some embodiments of the present invention, such as, but not limited to, those described in paragraphs [0170] and [0171], concurrent administration of fiber comprises administration 0.01 to 10 mg/kg of fiber.
Paragraph [0173] In some embodiments of the present invention, such as, but not limited to, those described in paragraphs [0170]-[0172], the fiber administered comprises one or more oligosaccharides, resistant starch, pectin, one or more beta-glucans, one or more xylooligosaccharides, or a combination thereof.
Paragraph [0174] In some embodiments of the present invention, such as, but not limited to, those described in paragraph [0173], the fiber administered comprises one or more oligosaccharides, at least one being a fructan, at least one being a galactan, or at least one being a fructan and at least one being a galactan.
Paragraph [0175] In some embodiments of the present invention, such as, but not limited to, those described in paragraphs [0154]-[0174], the non-human mammal is a ferret.
Paragraph [0176] In some embodiments of the present invention, such as, but not limited to, those described in paragraphs [0154]-[0174], the non-human mammal is a cat.
Paragraph [0177] In some embodiments of the present invention, such as, but not limited to, those described in paragraphs [0168]-[0174], the non-human mammal is a cat, and if administration is concurrent with consumption of high fiber food, the high fiber food has a fiber content of at least 3.5% by weight.
Paragraph [0178] In some embodiments of the present invention, such as, but not limited to, those described in paragraphs [0176] and [0177], the administration results in a significant change in at least one of the following microorganisms of the microbiota of the patient: Blautia, Oscillospira, Ruminococcus, Lachnospiraceae g1, Clostridiales f1.
Paragraph [0179] In some embodiments of the present invention, such as, but not limited to, those described in paragraph [0178], the administration results in a significant change in at least Blautia.
Paragraph [0180] In some embodiments of the present invention, such as, but not limited to, those described in paragraph [0178], the administration results in a significant change in at least Oscillospira.
Paragraph [0181] In some embodiments of the present invention, such as, but not limited to, those described in paragraph [0178], the administration results in a significant change in at least Ruminococcus.
Paragraph [0182] In some embodiments of the present invention, such as, but not limited to, those described in paragraph [0178], the administration results in a significant change in at least Lachnospiraceae g1.
Paragraph [0183] In some embodiments of the present invention, such as, but not limited to, those described in paragraph [0178], the administration results in a significant change in at least Clostridiales f1.
Paragraph [0184] In some embodiments of the present invention, such as, but not limited to, those described in paragraphs [0154]-[0174], the non-human mammal is a dog.
Paragraph [0185] In some embodiments of the present invention, such as, but not limited to, those described in paragraphs [0168]-[0174], the non-human mammal is a dog, and if administration is concurrent with consumption of high fiber food, the high fiber food has a fiber content of at least 5.0% by weight.
Paragraph [0186] In some embodiments of the present invention, such as, but not limited to, those described in paragraphs [0184] and [0185], the administration results in a significant change in at least one of the following microorganisms of the microbiota of the patient: Bacteroides, Enterococcus, Streptococcus, Ruminococcus, Blautia, Dorea, Prevotella, Clostridiaceae g1, Lachnospiraceae g1, Coprococcus, Oscillospira, Eubacterium, Clostridiales f1.
Paragraph [0187] In some embodiments of the present invention, such as, but not limited to, those described in paragraph [0186], the administration results in a significant change in at least Bacteroides.
Paragraph [0188] In some embodiments of the present invention, such as, but not limited to, those described in paragraph [0186], the administration results in a significant change in at least Enterococcus.
Paragraph [0189] In some embodiments of the present invention, such as, but not limited to, those described in paragraph [0186], the administration results in a significant change in at least Streptococcus.
Paragraph [0190] In some embodiments of the present invention, such as, but not limited to, those described in paragraph [0186], the administration results in a significant change in at least Ruminococcus.
Paragraph [0191] In some embodiments of the present invention, such as, but not limited to, those described in paragraph [0186], the administration results in a significant change in at least Blautia.
Paragraph [0192] In some embodiments of the present invention, such as, but not limited to, those described in paragraph [0186], the administration results in a significant change in at least Dorea.
Paragraph [0193] In some embodiments of the present invention, such as, but not limited to, those described in paragraph [0186], the administration results in a significant change in at least Prevotella.
Paragraph [0194] In some embodiments of the present invention, such as, but not limited to, those described in paragraph [0186], the administration results in a significant change in at least Clostridiaceae g1.
Paragraph [0195] In some embodiments of the present invention, such as, but not limited to, those described in paragraph [0186], the administration results in a significant change in at least, Lachnospiraceae g1.
Paragraph [0196] In some embodiments of the present invention, such as, but not limited to, those described in paragraph [0186], the administration results in a significant change in at least Coprococcus.
Paragraph [0197] In some embodiments of the present invention, such as, but not limited to, those described in paragraph [0186], the administration results in a significant change in at least Oscillospira.
Paragraph [0198] In some embodiments of the present invention, such as, but not limited to, those described in paragraph [0186], the administration results in a significant change in at least Eubacterium.
Paragraph [0199] In some embodiments of the present invention, such as, but not limited to, those described in paragraph [0186], the administration results in a significant change in at least Clostridiales f1.

### Examples

The following examples are given to aid in understanding the invention, but it is to be understood that the invention is not limited to the particular materials or procedures of the examples.

### Example 1A: Identification of Microorganisms in Microbiota of Potential Donors

Fecal material samples were obtained from three potential feline donors. The three donors were healthy, indoor, female cats, aged 3-9 years. The three donors' samples were screened for, and were free of, the following: Clostridium difficile toxins A and B, Cryptosporidium spp, Salmonella spp, Giardia spp, feline coronavirus, feline parvovirus (Panleukopenia), canine parvovirus 2, and Tritrichomonas foetus.

The three fecal material samples from the donors were screened by sequencing bacterial diversity based on the V4 hypervariable region of 16S rRNA. After performing PCR for this marker gene, libraries were sequenced using an Illumina MiSeq system, generating 250 bp paired-end amplicon reads. The amplicon data was multiplexed using dual barcode combinations for each sample. After sequencing, the samples were demultiplexed, filtered based on quality scores (FASTQ) and chimeras removed. Samples were then characterized for taxonomic composition (number and abundance) using Quantitative Insights in Microbial Ecology (QIIME v.1.9.1) by clustering at the >97 percent identity level and assigned taxonomic identification using QIIME's pick_otus_through_otu_table.py script. Specifically, a custom script was used to assign each pair of sequencing reads to their respective samples when parsing the raw data. This script allows for a 1-bp difference per barcode. The paired reads were then aligned and a consensus was computed using Fast Length Adjustment of SHort reads (FLASH) with a maximum overlap of 120 and a minimum overlap of 70 (other parameters were left as the default). The custom script automatically demultiplexes the data into fastq files, executes FLASH, and parses its results to reformat the sequences with appropriate naming conventions for software such as Quantitative Insights in Microbial Ecology (QIIME v.1.9.1; 46) in FASTA format. FASTA format is a text-based representation of nucleotide sequences in which base pairs are represented using single-letter codes.

Then each sample was binned using USEARCH and assigned taxonomic classification using RDP Classifier and the GreenGenes and/or Silva databases. Taxonomic assignments were confirmed using BLAST (Basic Local Alignment Search Tool).

The results of this screening analysis are presented below in Table 1:

TABLE 1
Bacterial diversity in three feline donors.

| Taxon | Fiona | Fabiola | Lola |
|---|---|---|---|
| Prevotellaceae. | 0.0004 | 0.000186608 | 0.373557677 |
| Prevotella | | | |
| Fusobacterium | 0.3332 | 0.000124405 | 0.153702609 |
| Paraprevotellaceae. | 0 | 0 | 0.090691382 |
| Prevotella | | | |
| Clostridiaceae. | 0.0884 | 0.027244736 | 0.087954468 |
| Clostridium | | | |
| Bacteroides | 0.1754 | 0.107641589 | 0.063384443 |
| Sutterella | 0.0012 | 0.003794358 | 0.051192735 |
| Catenibacterium | 0.0002 | 0.0000311 | 0.050104189 |
| Blautia | 0.0164 | 0.224520262 | 0.029888346 |
| Phascolarctobacterium | 0 | 0.0000311 | 0.025440861 |
| Faecalibacterium | 0 | 0.024601126 | 0.024010201 |
| Lachnospiraceae g2 | 0.0022 | 0.036855037 | 0.022703947 |
| Collinsella | 0.0014 | 0.210369172 | 0.009952415 |
| Oscillospira | 0 | 0.005909246 | 0.002954623 |
| Ruminococcus | 0 | 0.021708705 | 0.002145989 |
| Ruminococcus | 0 | 0.015301838 | 0.002145989 |
| Dorea | 0.096 | 0.003141231 | 0.002114888 |
| Ruminococcus | 0 | 0.021708705 | 0.001959382 |
| Ruminococcus | 0 | 0.015301838 | 0.001959382 |
| Clostridiaceae g1 | 0 | 0.00024881 | 0.001275153 |
| Parabacteroides | 0 | 0 | 0.001181849 |
| Erysipelotrichaceae g1 | 0 | 0.04699406 | 0.001057444 |
| Ruminococcaceae g1 | 0 | 0.039716356 | 0.00096414 |
| Clostridiales f2 | 0.0022 | 0.121170653 | 0.000870836 |
| Megasphaera | 0 | 0 | 0.00071533 |
| Lachnospiraceae g1 | 0.0464 | 0.006313563 | 0.000684229 |
| Enterococcus | 0.029 | 0.0000311 | 0.000373216 |
| Clostridiaceae g2 | 0.05 | 0.000155506 | 0.000311013 |
| Eubacterium | 0.0212 | 0.009392592 | 0.000186608 |
| Turicibacter | 0 | 0 | 0.000186608 |
| Slackia | 0.0002 | 0.00096414 | 0.0000622 |
| Streptococcus | 0.0054 | 0.000684229 | 0.0000622 |
| Lactobacillus | 0.0012 | 0.0000933 | 0.0000622 |
| Mogibacteriaceae g1 | 0 | 0 | 0.0000622 |
| Peptostreptococcaceae | 0 | 0 | 0.0000622 |
| g2 | | | |
| Coprococcus | 0 | 0.028115572 | 0.0000311 |
| Enterobacteriaceae g1 | 0 | 0.0000311 | 0.0000311 |
| Peptostreptococcus | 0.0418 | 0 | 0.0000311 |
| Clostridiaceae.SMB53 | 0 | 0 | 0.0000311 |
| Bifidobacterium | 0.0012 | 0.03166112 | 0 |
| Megamonas | 0.009 | 0.025627469 | 0 |
| Lachnospira | 0 | 0.003203434 | 0 |
| Anaerobiospirillum | 0 | 0.001306254 | 0 |
| Helicobacter | 0 | 0.000933039 | 0 |
| Succinivibrionaceae g1 | 0 | 0.000870836 | 0 |
| Clostridiales f1 | 0 | 0.000622026 | 0 |
| Roseburia | 0 | 0.000311013 | 0 |
| Holdemania | 0 | 0.0000622 | 0 |
| Bacillus | 0 | 0.0000311 | 0 |
| Lachnospiraceae. | 0.0496 | 0 | 0 |
| Ruminococcus | | | |
| Peptoniphilus | 0.0064 | 0 | 0 |
| Coriobacteriaceae g2 | 0.0048 | 0 | 0 |
| Enterobacteriaceae g2 | 0.0038 | 0 | 0 |
| Veillonella | 0.0028 | 0 | 0 |
| Coriobacteriaceae g1 | 0.0022 | 0 | 0 |
| Sharpea | 0.0022 | 0 | 0 |
| Peptococcus | 0.0012 | 0 | 0 |
| Erysipelotrichaceae g2 | 0.0012 | 0 | 0 |
| Halomonas | 0.0008 | 0 | 0 |
| Vibrionaceae g2 | 0.0006 | 0 | 0 |
| Lactococcus | 0.0004 | 0 | 0 |
| Peptostreptococcaceae g1 | 0.0002 | 0 | 0 |
| Leuconostoc | 0.0002 | 0 | 0 |
| Enterococcaceae g1 | 0.0002 | 0 | 0 |
| Lactobacillales f1 | 0.0002 | 0 | 0 |
| Macrococcus | 0.0002 | 0 | 0 |
| Stramenopiles f1 | 0.0002 | 0 | 0 |
| Bacteria OP9 JS1 SB-45 f1 | 0.0002 | 0 | 0 |
| Porphyromonas | 0.0002 | 0 | 0 |

### Example 1B

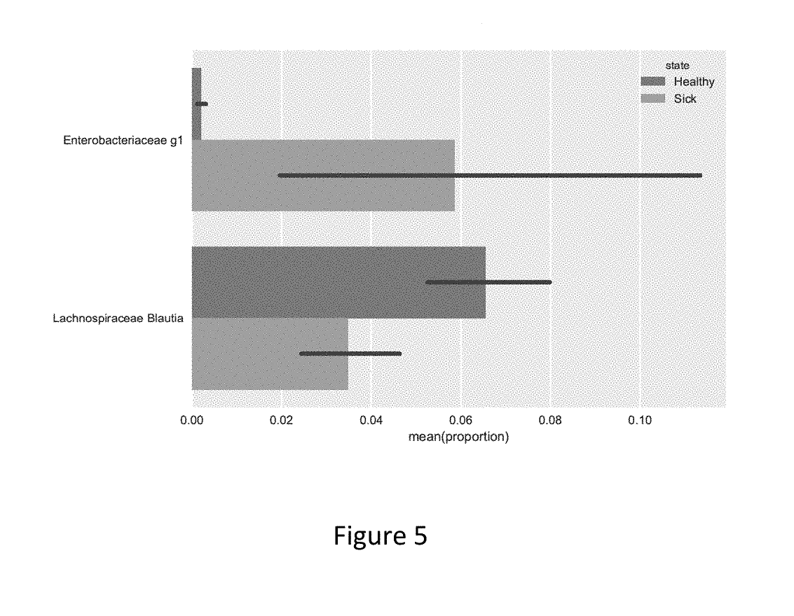

In a manner similar to that described in Example 1A, fecal samples of 81 healthy domestic cats and 48 unhealthy domestic cats (cats with a gastrointestinal disease or disorder) were screened. The results of this screening analysis indicated that healthy domestic cat donors (n=81) have a significantly higher proportion of Lachnospiraceae Blautia (P=0.00115, DF=117) while unhealthy potential donors (n=48) have a significantly higher proportion of Enterobacteriaceae g1 (P=0.0345, DF=23) (see FIG. 5).

### Example 2: Preparation of a Solid Oral Dosage Form

A sample of fecal material from a donor screened as described in Example 1A and free of the above listed pathogens was cleaned of any outside contamination, such as cat litter. After cleaning, the sample or a portion of the sample was weighed, and a cryoprotectant, glycerol (vegetable glycerol), was added at a minimum of 20% by weight (5 parts by weight fecal material to 1 part or more by weight cryoprotectant). The fecal material was mixed with the cryoprotectant, and then flattened on parchment paper before freeze drying. After drying, the freeze-dried material was subjected to size reduction by grinding with a coffee grinder. The resulting powder was filled into capsules, which were subsequently coated with an enteric coating, specifically, edible shellac.

### Example 3: Clinical Assessment

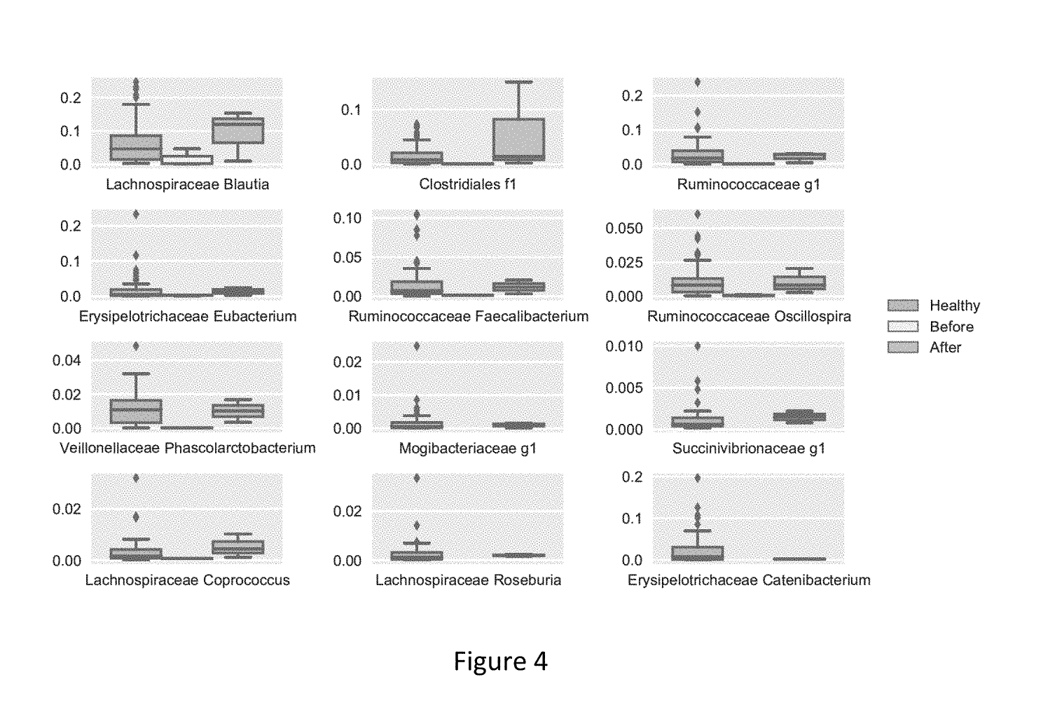

Capsules including microorganisms from feline donors were screened in a similar manner as that described in Example 1 and solid oral dosage forms were prepared in a manner similar to Example 2. A number of feline subjects/recipients with gastrointestinal disorders or diseases were treated with oral fecal microbiota transplant capsules (the study is on-going). The fecal microbiota of the recipients was screened and characterized as described in Example 1 both before and after treatment. Results of the identification of the microbiota for the animals before and after treatment for three of the animals who have completed treatment (and for whom data analysis has been completed) as compared to healthy animals (n=81; same as Example 1B) are illustrated in FIG. 4, where the left most bar is the data for the healthy animals, the middle bar is the data for the treatment animals/study subjects before treatment, and the rightmost bar represents the data for the treatment animals/study subjects after treatment.

### Example 4—Pilot Study in Cats and Dogs

Capsules including microorganisms from canine donors were screened in a manner similar to that described in Example 1 and solid oral dosage forms, referred to as Fecal Microbiota Transplant (FMT) capsules, were prepared in a manner similar to that described in Example 2. Various social media platforms were used to recruit people with dogs and cats exhibiting symptoms of a chronic digestive condition (diarrhea, vomiting, and/or constipation), who received a pilot study kit. Pilot study kits contained 50 FMT capsules, a health survey, and materials to collect two fecal samples. Participants gave one to two capsules to their dog or cat orally with food each day for ˜25 days, which is referred to as FMT treatment. Participants were also asked to collect fecal samples “before” and “after” the course of capsules, with the “after” samples collected two weeks after the course of treatment ended.

### Description of the Survey

The survey provided in the pilot study kits was designed to capture owners' observations while their pets were taking the FMT capsules. The owners recorded physical descriptions and lifestyle information about each animal including age, breed, gender, and diet. In addition, owners were asked to score their pet's body condition, on a scale ranging from 1 (severely underweight) to 10 (severely overweight), with 5 considered a healthy body condition. Owners were also asked to provide a general health description of their animal, including diagnoses they had received from veterinarians. The owners recorded the specific symptoms they were hoping to alleviate with FMT treatment. Specific symptoms listed included diarrhea, constipation, vomiting, and lack of appetite. Owners were asked to record the typical fecal consistency of their animal prior to beginning the capsules and after completing the course of treatment with the capsules, following the Bristol Stool Scale, which ranges from 1 for a hard and constipated stool, to 7 for a watery diarrhea, with 3 and 4 considered normal. (Lewis and Heaton 1997.) Owners were provided pictures to guide their ratings of the stool consistency, and were also asked to provide photo documentation. Following the course of capsules, owners were asked to evaluate whether they considered the oral FMT capsules successful overall by selecting one of the following options: clear success, some improvement, no change, or worsening clinical signs.

### Results—Dogs

Twenty-eight dog owners participated and provided surveys, but the overall data set was filtered to remove dogs that were later determined to have cancer, or whose original symptoms did not include gastrointestinal issues. After filtering, the final data set included information for 21 dogs.

All 21 dogs included in the analysis were having chronic gastrointestinal issues manifesting in chronic diarrhea and/or vomiting and/or constipation, although the underlying conditions were not always clear. Some dogs had Inflammatory Bowel Disease (diagnosed or suspected). Dog breeds included in this pilot study were Akita, Beagle Mix (Shepherd), Bichon Poodle, Border Collie, Boxer mix, Bull Terrier, Chihuahua Mix, Cocker spaniel mix—Papillor and Cavalier King Charles, German shepherd, German Short-haired Pointer, Golden Retriever mix, Golden Retriever, Goldendoodle, Great Dane, McNab Shepherd, Mini Schnauzer, Papillon, Pitbull mix, Whippet, Shepherd mix, Chow, Corgi, and Poodle.

At the beginning of the oral FMT treatment, the mean body condition for dogs was 4.95 (standard deviation: 1.21). The mean age of the dogs was 6.4 years (standard deviation: 4.43), with an age range of 1 to 15 years old. Of the dogs included in this study, 54% were female and 46% were male. Mean fecal consistency before FMT capsules for dogs with chronic diarrhea shifted from 5.5 (soft blobs) on the Bristol Stool Chart, to 3 (normal) after completing the full course of FMT capsules. Mean fecal consistency for dogs with chronic constipation was 1 on the Bristol Stool Chart (separate hard lumps), which shifted towards normal to 2.3 (sausage shaped) after the FMT capsules.

Of the 21 canine patients included, 89% of study participants reported some or clear improvement in symptoms associated with chronic gastrointestinal issues and 68% reported a clear improvement, while 5% reported that no change and 5% reported that clinical signs worsened. For the fifteen study participants owning a dog with chronic diarrhea, 86% of study participants reported some improvement with chronic diarrhea, 73% reported a clear improvement, 7% reported no change, and 7% reported that clinical signs worsened. All four (100%) of the study participants owning a dog with chronic vomiting reported some improvement with chronic vomiting, and three of the four (75%) reported a clear improvement. For the three study participants owning a dog with chronic constipation, two (67%) of reported a clear improvement, while one (33%) reported an increase in constipation.

Table 2 summarizes the data for the dogs in the pilot study described above.

TABLE 2
Change in clinical signs in Dogs who completed a full
course of 50 capsules.

| | Overall | Diarrhea | Vomiting | Constipation |
|---|---|---|---|---|
| | (21) | (15) | (4) | (3) |
| Clear | 0.68 | 0.73 | 0.75 | 0.67 |
| improvement | | | | |
| Some | 0.21 | 0.13 | 0.25 | |
| improvement | | | | |
| No change | 0.05 | 0.07 | | |
| Clinical signs | 0.05 | 0.07 | | 0.33 |
| worsened | | | | |

### Results—Cats

Sixty three cat owners participated and provided surveys, but the overall data set was filtered to remove cats that were later determined to have cancer, or whose original symptoms did not include gastrointestinal issues. After filtering, the final data set included information for 55 cats.

All 55 cats included in the analysis were having chronic gastrointestinal issues manifesting in chronic diarrhea and/or vomiting and/or constipation, though as with the dogs, the underlying conditions were not always clear. Some cats had Inflammatory Bowel Disease (diagnosed or suspected) and some had, in addition to chronic gastrointestinal issues, pancreatitis or kidney disease. Cat breeds included in the study were American shorthair, American medium hair, Bengal, Burmese, Domestic long hair, Domestic long hair/Maine Coon mix, Domestic medium hair, Domestic short hair, Norwegian Forest cat, Ragdoll, Siamese mix, and Siberia.

At the beginning of the oral FMT treatment, the mean body condition for cats was 4.81 (standard deviation: 1.78). The mean age of the cats was 9.8 years (standard deviation: 5.1). The ages ranged from 0.4 to 19 years old. Of the cats included in this study, 43% were female and 57% were male. Mean fecal consistency before FMT capsules for cats with chronic diarrhea shifted from 5.8 (soft blobs) on the Bristol Stool Chart, to 4 (normal) after completing the full course of FMT capsules. Mean fecal consistency for cats with chronic constipation was 1.6 on the Bristol Stool Chart (separate hard lumps), which shifted towards normal to 2.7 (sausage shaped) after the FMT capsules.

Of the 55 cat patients included, 89% of study participants reported some or clear improvement in overall clinical signs associated with chronic gastrointestinal issues and 68% reported a clear improvement, while 9% reported no change and 2% reported that clinical signs worsened. For the study participants with a cat with chronic diarrhea, 75% of study participants reported some improvement, 59% reported a clear improvement, 19% reported no change, and 5% reported a worsening in clinical signs. For the study participants with a cat with chronic vomiting, 72% of study participants reported some improvement and 48% reported a clear improvement, 24% reported no change, and 4% reported that clinical signs worsened. For the study participants with a cat with chronic constipation (n=12), 67% of study participants reported some improvement, 42% reported a clear improvement, and 33% reported no change.

Table 3 summarizes the data for the cats in the pilot study described above.

TABLE 3
Change in clinical signs in cats who completed a full
course of 50 capsules.

| | Overall | Diarrhea | Vomiting | Constipation |
|---|---|---|---|---|
| | (55) | (37) | (25) | (12) |
| Clear improvement | 0.68 | 0.59 | 0.48 | 0.42 |
| Some improvement | 0.21 | 0.16 | 0.24 | 0.25 |
| No change | 0.09 | 0.19 | 0.24 | 0.33 |
| Clinical signs | 0.02 | 0.05 | 0.04 | |
| worsened | | | | |

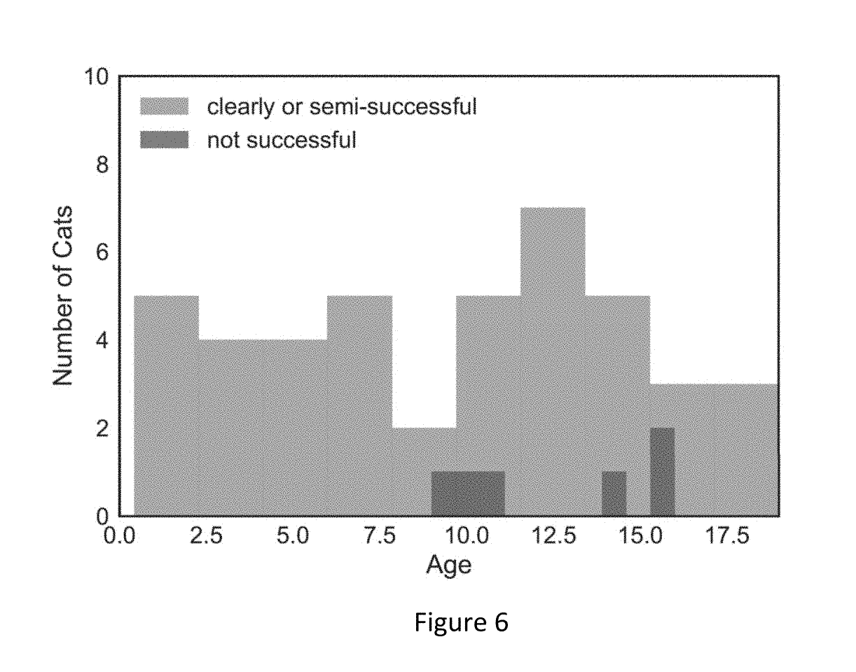

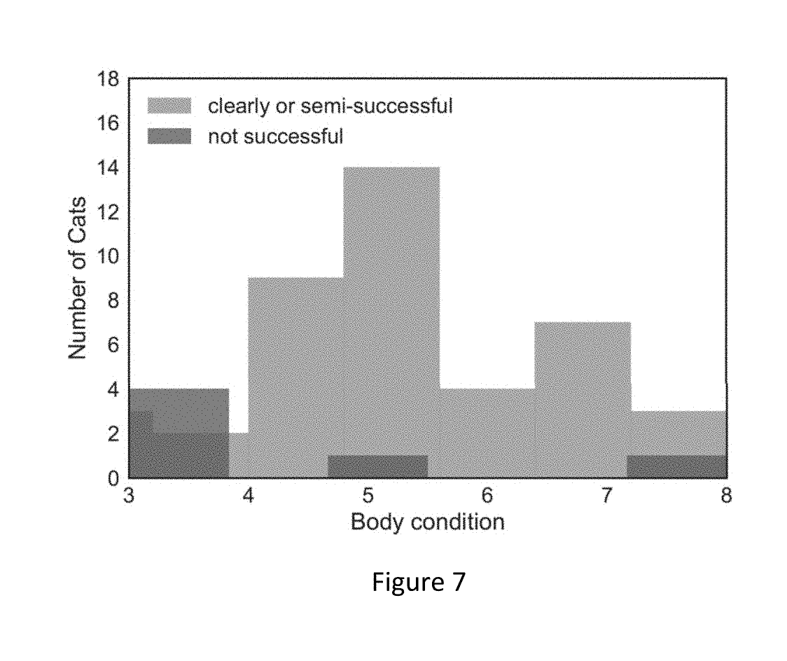

FIG. 6 is a bar chart comparing semi-successful or successful cases to the unsuccessful cases as a function of the feline study participant's age. As seen in FIG. 6, the unsuccessful cases are older cats. FIG. 7 is a bar chart comparing semi-successful or successful cases to the unsuccessful cases as a function of the feline study participant's body condition. As seen in FIG. 7, for the cats with a lower body weight, there were more unsuccessful cases than successful cases. Low body weight may be indicative of additional health problems.

### Example 5—Comparison of the Microbiome Before and after Treatment

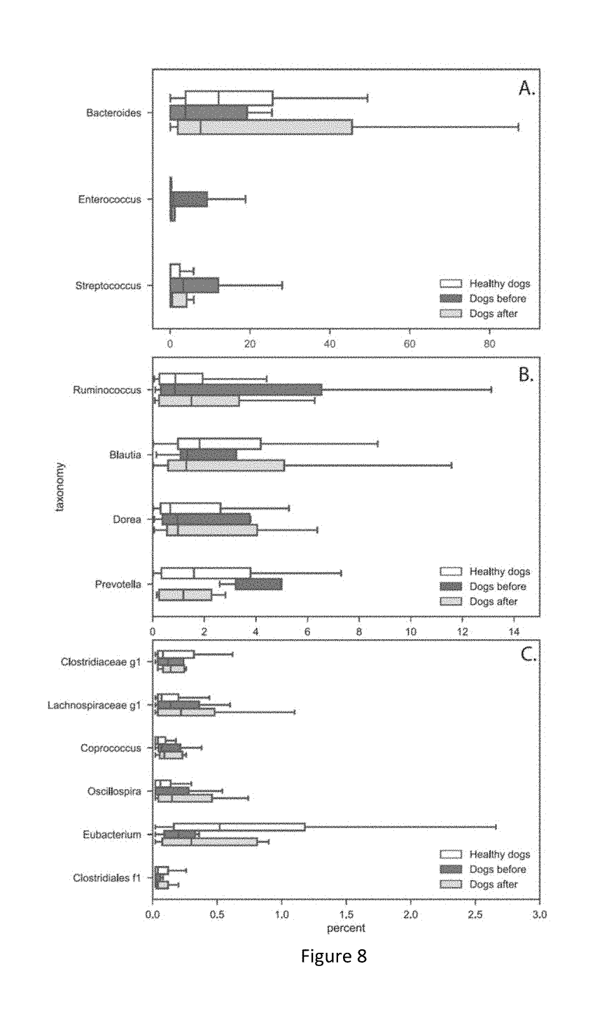

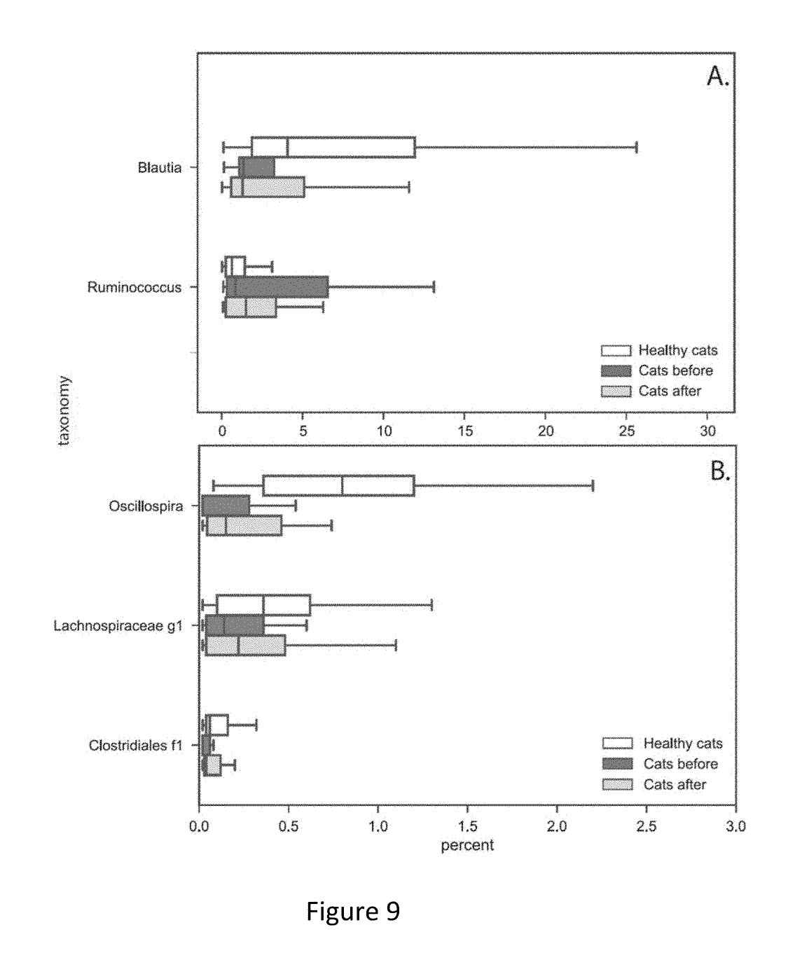

FIG. 8 illustrates those bacterial taxa that differed significantly (P<0.05), from before treatment to after treatment, in 16 domestic dogs receiving one or two capsules a day with food for about 25 days. Fecal samples were collected before and after each dog received the course of treatment. The bacteria are divided into three groups labeled “A,” “B,” and “C,” having different abundancies, and thus each group has a different scale. Significance testing was performed using the group significance test in QIIME 1.9 (QIIME: J Gregory Caporaso et al.; QIIME allows analysis of high-throughput community sequencing data; Nature Methods, 2010; doi:10.1038/nmeth.f.303). The 16 dogs are a subset of the 21 dogs in the study for whom we received fecal samples both before and after the dog completed the full course of 50 FMT capsules. FIG. 9 illustrates those bacterial taxa that differed significantly (P<0.05), from before treatment to after treatment, in 40 domestic cats for whom we received fecal samples both before and after the cat completed the full course of 50 FMT capsules. A full course of the oral capsules is one or two capsules a day with food for about 25 days. Fecal samples were collected before and after each cat received the oral fecal transplant. The bacteria are divided into two groups labeled “A,” and “B,” having different abundancies. Significance testing was performed using the group significance test in QIIME 1.9.

### Example 6—Compilation of Identified Microorganisms of Potential Donors

Tables 4 and 5 provide a summary of the identified microorganisms from the fecal matter of 93 canine and 85 feline donors.

TABLE 4
Identified Microorganisms in Dogs

| | % ind'ls | Mean % | Highest % |
|---|---|---|---|
| Phylum: Class: Order: Family: Genus | in popn | w/in ind'l | w/in ind'l |
| Firmicutes: Clostridia: Clostridiales: Lachnospiraceae: | 100 | 0.0415 | 0.1041 |
| Blautia | | | |
| Firmicutes: Clostridia: Clostridiales: Lachnospiraceae: | 100 | 0.0334 | 0.0756 |
| none | | | |
| Firmicutes: Clostridia: Clostridiales: Clostridiaceae: none | 100 | 0.0245 | 0.0518 |
| Firmicutes: Clostridia: Clostridiales: Lachnospiraceae: | 100 | 0.0226 | 0.0598 |
| [Ruminococcus] | | | |
| Fusobacteria: Fusobacteriia: Fusobacteriales: | 98.9 | 0.2607 | 0.4925 |
| Fusobacteriaceae: Fusobacterium | | | |
| Firmicutes: Clostridia: Clostridiales: Lachnospiraceae: | 98.9 | 0.0217 | 0.0541 |
| Dorea | | | |
| Firmicutes: Clostridia: Clostridiales: none: none | 97.8 | 0.0082 | 0.0258 |
| Bacteroidetes: Bacteroidia: Bacteroidales: | 95.7 | 0.1699 | 0.3377 |
| Bacteroidaceae: Bacteroides | | | |
| Firmicutes: Erysipelotrichi: Erysipelotrichales: | 89.1 | 0.0092 | 0.0235 |
| Erysipelotrichaceae: [Eubacterium] | | | |
| Proteobacteria: Betaproteobacteria: Burkholderiales: | 89.1 | 0.008 | 0.0223 |
| Alcaligenaceae: Sutterella | | | |
| Actinobacteria: Coriobacteriia: Coriobacteriales: | 88 | 0.0072 | 0.0314 |
| Coriobacteriaceae: Collinsella | | | |
| Firmicutes: Erysipelotrichi: Erysipelotrichales: | 87 | 0.0066 | 0.0232 |
| Erysipelotrichaceae: none | | | |
| Firmicutes: Clostridia: Clostridiales: Veillonellaceae: | 81.5 | 0.0721 | 0.1803 |
| Megamonas | | | |
| Firmicutes: Clostridia: Clostridiales: Ruminococcaceae: | 81.5 | 0.0039 | 0.0137 |
| none | | | |
| Bacteroidetes: Bacteroidia: Bacteroidales: | 79.3 | 0.1814 | 0.4437 |
| Prevotellaceae: Prevotella | | | |
| Firmicutes: Clostridia: Clostridiales: Clostridiaceae: | 79.3 | 0.0025 | 0.0111 |
| Clostridium | | | |
| Firmicutes: Clostridia: Clostridiales: Clostridiaceae: | 77.2 | 0.004 | 0.0158 |
| Other | | | |
| Firmicutes: Clostridia: Clostridiales: Lachnospiraceae: | 73.9 | 0.001 | 0.0025 |
| Other | | | |
| Firmicutes: Clostridia: Clostridiales: Ruminococcaceae: | 72.8 | 0.014 | 0.0464 |
| Faecalibacterium | | | |
| Firmicutes: Bacilli: Lactobacillales: Streptococcaceae: | 60.9 | 0.0256 | 0.1044 |
| Streptococcus | | | |
| Firmicutes: Erysipelotrichi: Erysipelotrichales: | 60.9 | 0.0061 | 0.0225 |
| Erysipelotrichaceae: Allobaculum | | | |
| Other: Other: Other: Other: Other | 57.6 | 0.0005 | 0.0013 |
| Firmicutes: Clostridia: Clostridiales: Lachnospiraceae: | 56.5 | 0.0005 | 0.0013 |
| Coprococcus | | | |
| Bacteroidetes: Bacteroidia: Bacteroidales: | 55.4 | 0.0176 | 0.0526 |
| [Paraprevotellaceae]: [Prevotella] | | | |
| Proteobacteria: Gammaproteobacteria: Enterobacteriales: | 55.4 | 0.0037 | 0.0146 |
| Enterobacteriaceae: none | | | |
| Firmicutes: Clostridia: Clostridiales: Veillonellaceae: | 44.6 | 0.0036 | 0.0103 |
| Phascolarctobacterium | | | |
| Firmicutes: Erysipelotrichi: Erysipelotrichales: | 43.5 | 0.0173 | 0.0698 |
| Erysipelotrichaceae: Catenibacterium | | | |
| Firmicutes: Clostridia: Clostridiales: Ruminococcaceae: | 43.5 | 0.0003 | 0.0009 |
| Ruminococcus | | | |
| Firmicutes: Erysipelotrichi: Erysipelotrichales: | 39.1 | 0.001 | 0.0033 |
| Erysipelotrichaceae: Coprobacillus | | | |
| Firmicutes: Bacilli: Lactobacillales: Lactobacillaceae: | 37 | 0.0039 | 0.0376 |
| Lactobacillus | | | |
| Firmicutes: Clostridia: Clostridiales: | 37 | 0.0023 | 0.012 |
| Peptostreptococcaceae: none | | | |
| Actinobacteria: Coriobacteriia: Coriobacteriales: | 37 | 0.0002 | 0.0007 |
| Coriobacteriaceae: Slackia | | | |
| Firmicutes: Clostridia: Clostridiales: Other: Other | 35.9 | 0.0006 | 0.003 |
| Firmicutes: Clostridia: Clostridiales: | 34.8 | 0.0027 | 0.0156 |
| Peptostreptococcaceae: Other | | | |
| Firmicutes: Bacilli: Turicibacterales: Turicibacteraceae: | 34.8 | 0.0024 | 0.0098 |
| Turicibacter | | | |
| Firmicutes: Clostridia: Clostridiales: Ruminococcaceae: | 33.7 | 0.0004 | 0.0017 |
| Oscillospira | | | |
| Firmicutes: Bacilli: Lactobacillales: Enterococcaceae: | 26.1 | 0.0007 | 0.0037 |
| Enterococcus | | | |
| Proteobacteria: Gammaproteobacteria: Aeromonadales: | 25 | 0.0005 | 0.003 |
| Succinivibrionaceae: none | | | |
| Bacteroidetes: Bacteroidia: Bacteroidales: | 22.8 | 0.0005 | 0.0024 |
| Porphyromonadaceae: Parabacteroides | | | |
| Firmicutes: Clostridia: Clostridiales: Lachnospiraceae: | 22.8 | 0.0001 | 0.0007 |
| Roseburia | | | |
| Firmicutes: Clostridia: Clostridiales: Peptococcaceae: | 21.7 | 0.0006 | 0.0021 |
| Peptococcus | | | |
| Proteobacteria: Gammaproteobacteria: Aeromonadales: | 20.7 | 0.0012 | 0.0055 |
| Succinivibrionaceae: Anaerobiospirillum | | | |
| Bacteroidetes: Bacteroidia: Bacteroidales: S24-7: none | 20.7 | 0.0003 | 0.0013 |
| Actinobacteria: Actinobacteria: Bifidobacteriales: | 16.3 | 0.0005 | 0.0027 |
| Bifidobacteriaceae: Bifidobacterium | | | |
| Proteobacteria: Epsilonproteobacteria: | 15.2 | 0.0003 | 0.0015 |
| Campylobacterales: Campylobacteraceae: | | | |
| Campylobacter | | | |
| Firmicutes: Clostridia: Clostridiales: Veillonellaceae: | 14.1 | 0.0011 | 0.0068 |
| Megasphaera | | | |
| Firmicutes: Clostridia: Clostridiales: Clostridiaceae: | 12 | 0.0011 | 0.0068 |
| Candidatus Arthromitus | | | |
| Firmicutes: Clostridia: Clostridiales: Veillonellaceae: | 12 | 0.0003 | 0.0018 |
| Dialister | | | |
| Tenericutes: Mollicutes: Anaeroplasmatales: | 12 | 0.0003 | 0.0017 |
| Anaeroplasmataceae: Anaeroplasma | | | |
| Bacteroidetes: Bacteroidia: Bacteroidales: Other: Other | 12 | 0.0001 | 0.0003 |
| Firmicutes: Bacilli: Lactobacillales: Streptococcaceae: | 12 | 0.0001 | 0.0004 |
| Lactococcus | | | |
| Firmicutes: Bacilli: Lactobacillales: Enterococcaceae: | 10.9 | 0.0001 | 0.0008 |
| Other | | | |
| Proteobacteria: Epsilonproteobacteria: | 10.9 | 0.0001 | 0.0002 |
| Campylobacterales: Helicobacteraceae Helicobacter | | | |
| Firmicutes: Bacilli: Bacillales: Bacillaceae: Bacillus | 10.9 | 0 | 0.0001 |
| Firmicutes: Clostridia: Clostridiales: Clostridiaceae: | 9.8 | 0.0061 | 0.0626 |
| Sarcina | | | |
| Bacteroidetes: Bacteroidia: Bacteroidales: | 9.8 | 0 | 0.0001 |
| Bacteroidaceae: 5-7N15 | | | |
| Bacteroidetes: Bacteroidia: Bacteroidales: | 8.7 | 0.0033 | 0.0255 |
| [Paraprevotellaceae]: none | | | |
| Proteobacteria: Gammaproteobacteria: Oceanospirillales: | 8.7 | 0.0001 | 0.0011 |
| Halomonadaceae: Halomonas | | | |
| Acidobacteria: Acidobacteriia: Acidobacteriales: | 8.7 | 0 | 0.0001 |
| Acidobacteriaceae: none | | | |
| Firmicutes: Bacilli: Bacillales: Staphylococcaceae: | 8.7 | 0 | 0.0001 |
| Staphylococcus | | | |
| Proteobacteria: Alphaproteobacteria: Rhodospirillales: | 8.7 | 0 | 0.0001 |
| Acetobacteraceae: none | | | |
| Firmicutes: Clostridia: Clostridiales: Lachnospiraceae: | 7.6 | 0.0001 | 0.0013 |
| Lachnospira | | | |
| Firmicutes: Bacilli: Lactobacillales: Other: Other | 7.6 | 0 | 0.0003 |
| Firmicutes: Bacilli: Lactobacillales: Leuconostocaceae: | 6.5 | 0 | 0.0001 |
| Leuconostoc | | | |
| Bacteroidetes: Bacteroidia: Bacteroidales: | 5.4 | 0.0001 | 0.0004 |
| [Paraprevotellaceae]: Paraprevotella | | | |
| Actinobacteria: Actinobacteria: Actinomycetales: | 5.4 | 0 | 0.0001 |
| Actinomycetaceae: Actinomyces | | | |
| Proteobacteria: Betaproteobacteria: Burkholderiales: | 5.4 | 0 | 0.0001 |
| Other: Other | | | |
| Firmicutes: Clostridia: Clostridiales: Lachnospiraceae: | 4.3 | 0.0002 | 0.0017 |
| Epulopiscium | | | |
| Actinobacteria: Coriobacteriia: Coriobacteriales: | 4.3 | 0 | 0.0001 |
| Coriobacteriaceae: Adlercreutzia | | | |
| Firmicutes: Bacilli: Gemellales: Gemellaceae: Gemella | 4.3 | 0 | 0 |
| Firmicutes: Clostridia: Clostridiales: [Mogibacteriaceae]: | 4.3 | 0 | 0.0003 |
| none | | | |
| Firmicutes: Clostridia: Clostridiales: Lachnospiraceae: | 4.3 | 0 | 0.0001 |
| Clostridium | | | |
| Firmicutes: Clostridia: Clostridiales: Veillonellaceae: | 4.3 | 0 | 0.0002 |
| Veillonella | | | |
| Firmicutes: Erysipelotrichi: Erysipelotrichales: | 4.3 | 0 | 0.0001 |
| Erysipelotrichaceae: Holdemania | | | |
| Proteobacteria: Gammaproteobacteria: Alteromonadales: | 4.3 | 0 | 0.0003 |
| Shewanellaceae: Shewanella | | | |
| Proteobacteria: Gammaproteobacteria: Enterobacteriales: | 4.3 | 0 | 0.0001 |
| Enterobacteriaceae: Other | | | |
| Proteobacteria: Gammaproteobacteria: | 4.3 | 0 | 0.0001 |
| Pseudomonadales: Pseudomonadaceae: Pseudomonas | | | |
| Actinobacteria: Actinobacteria: Actinomycetales: none: | 3.3 | 0 | 0.0001 |
| none | | | |
| Bacteroidetes: Bacteroidia: Bacteroidales: | 3.3 | 0 | 0.0001 |
| Bacteroidaceae: Other | | | |
| Bacteroidetes: Bacteroidia: Bacteroidales: none: none | 3.3 | 0 | 0.0001 |
| Firmicutes: Bacilli: Bacillales: Listeriaceae: Brochothrix | 3.3 | 0 | 0.0001 |
| Firmicutes: Bacilli: Bacillales: Listeriaceae: Other | 3.3 | 0 | 0 |
| Firmicutes: Bacilli: Lactobacillales: Enterococcaceae: | 3.3 | 0 | 0.0002 |
| Vagococcus | | | |
| Firmicutes: Bacilli: Lactobacillales: none: none | 3.3 | 0 | 0.0001 |
| Firmicutes: Clostridia: Clostridiales: Clostridiaceae: | 3.3 | 0 | 0 |
| SMB53 | | | |
| Fusobacteria: Fusobacteriia: Fusobacteriales: | 3.3 | 0 | 0.0001 |
| Fusobacteriaceae: Cetobacterium | | | |
| Fusobacteria: Fusobacteriia: Fusobacteriales: | 3.3 | 0 | 0.0001 |
| Fusobacteriaceae: Other | | | |
| Planctomycetes: Planctomycetia: Pirellulales: | 3.3 | 0 | 0 |
| Pirellulaceae: none | | | |
| Proteobacteria: Alphaproteobacteria: Rhodobacterales: | 3.3 | 0 | 0 |
| Rhodobacteraceae: none | | | |
| Proteobacteria: Gammaproteobacteria: Enterobacteriales: | 3.3 | 0 | 0.0001 |
| Enterobacteriaceae: Citrobacter | | | |
| Proteobacteria: Gammaproteobacteria: Oceanospirillales: | 3.3 | 0 | 0 |
| Endozoicimonaceae: none | | | |
| Proteobacteria: Deltaproteobacteria: Desulfovibrionales: | 2.2 | 0.0001 | 0.0008 |
| Desulfovibrionaceae: none | | | |
| Crenarchaeota: Thaumarchaeota: Nitrososphaerales: | 2.2 | 0 | 0 |
| Nitrososphaeraceae: Candidatus Nitrososphaera | | | |
| [Thermi]: Deinococci: Thermales: Thermaceae: | 2.2 | 0 | 0 |
| Meiothermus | | | |
| Actinobacteria: Actinobacteria: Actinomycetales: | 2.2 | 0 | 0 |
| Corynebacteriaceae: Corynebacterium | | | |
| Actinobacteria: Actinobacteria: Actinomycetales: | 2.2 | 0 | 0 |
| Micromonosporaceae: Couchioplanes | | | |
| Bacteroidetes: [Saprospirae]: [Saprospirales]: | 2.2 | 0 | 0.0001 |
| Chitinophagaceae: none | | | |
| Bacteroidetes: Bacteroidia: Bacteroidales: | 2.2 | 0 | 0.0001 |
| [Odoribacteraceae]: Odoribacter | | | |
| Bacteroidetes: Bacteroidia: Bacteroidales: Rikenellaceae: | 2.2 | 0 | 0 |
| none | | | |
| Chloroflexi: Dehalococcoidetes: Dehalococcoidales: | 2.2 | 0 | 0.0001 |
| Dehalococcoidaceae: none | | | |
| Chloroflexi: Dehalococcoidetes: GIF9: none: none | 2.2 | 0 | 0 |
| Cyanobacteria: 4C0d-2: YS2: none: none | 2.2 | 0 | 0.0002 |
| Firmicutes: Bacilli: Bacillales: Alicyclobacillaceae: | 2.2 | 0 | 0 |
| Alicyclobacillus | | | |
| Firmicutes: Bacilli: Bacillales: Planococcaceae: none | 2.2 | 0 | 0.0001 |
| Firmicutes: Bacilli: Lactobacillales: Lactobacillaceae: | 2.2 | 0 | 0 |
| Other | | | |
| Firmicutes: Bacilli: Lactobacillales: Leuconostocaceae: | 2.2 | 0 | 0.0001 |
| Weissella | | | |
| Firmicutes: Clostridia: Clostridiales: Eubacteriaceae: | 2.2 | 0 | 0 |
| Pseudoramibacter_Eubacterium | | | |
| Firmicutes: Clostridia: Clostridiales: | 2.2 | 0 | 0 |
| Peptostreptococcaceae: Peptostreptococcus | | | |
| Firmicutes: Clostridia: Clostridiales: Ruminococcaceae: | 2.2 | 0 | 0.0001 |
| Other | | | |
| Fusobacteria: Fusobacteriia: Fusobacteriales: | 2.2 | 0 | 0.0001 |
| Leptotrichiaceae: Leptotrichia | | | |
| Proteobacteria: Alphaproteobacteria: Rhizobiales: | 2.2 | 0 | 0 |
| Methylocystaceae: none | | | |
| Proteobacteria: Alphaproteobacteria: Rhodobacterales: | 2.2 | 0 | 0.0001 |
| Rhodobacteraceae: Paracoccus | | | |
| Proteobacteria: Alphaproteobacteria: Sphingomonadales: | 2.2 | 0 | 0 |
| Sphingomonadaceae: Sphingomonas | | | |
| Proteobacteria: Deltaproteobacteria: BPC076: none: none | 2.2 | 0 | 0 |
| Spirochaetes: Spirochaetes: Spirochaetales: | 2.2 | 0 | 0.0001 |
| Spirochaetaceae: none | | | |
| Tenericutes: Mollicutes: Anaeroplasmatales: | 2.2 | 0 | 0.0001 |
| Anaeroplasmataceae: gut | | | |
| Crenarchaeota: MCG: none: none: none | 1.1 | 0 | 0 |
| Crenarchaeota: MCG: pGrfC26: none: none | 1.1 | 0 | 0.0001 |
| Acidobacteria: Acidobacteriia: Acidobacteriales: | 1.1 | 0 | 0 |
| Acidobacteriaceae: Terriglobus | | | |
| Acidobacteria: Acidobacteriia: Acidobacteriales: | 1.1 | 0 | 0 |
| Koribacteraceae: Candidatus Koribacter | | | |
| Acidobacteria: Acidobacteriia: Acidobacteriales: | 1.1 | 0 | 0 |
| Koribacteraceae: none | | | |
| Acidobacteria: PAUC37f: none: none: none | 1.1 | 0 | 0 |
| Acidobacteria: Solibacteres: Solibacterales: AKIW659: | 1.1 | 0 | 0 |
| none | | | |
| Acidobacteria: Solibacteres: Solibacterales: none: none | 1.1 | 0 | 0 |
| Acidobacteria: Solibacteres: Solibacterales: | 1.1 | 0 | 0 |
| Solibacteraceae: none | | | |
| Acidobacteria: TM1: none: none: none | 1.1 | 0 | 0 |
| Actinobacteria: Actinobacteria: Actinomycetales: ACK- | 1.1 | 0 | 0 |
| M1: none | | | |
| Actinobacteria: Actinobacteria: Actinomycetales: | 1.1 | 0 | 0 |
| Geodermatophilaceae: Modestobacter | | | |
| Actinobacteria: Actinobacteria: Actinomycetales: | 1.1 | 0 | 0 |
| Gordoniaceae: Gordonia | | | |
| Actinobacteria: Actinobacteria: Actinomycetales: | 1.1 | 0 | 0 |
| Microbacteriaceae: Leucobacter | | | |
| Actinobacteria: Actinobacteria: Actinomycetales: | 1.1 | 0 | 0 |
| Micrococcaceae: Other | | | |
| Actinobacteria: Actinobacteria: Actinomycetales: Other: | 1.1 | 0 | 0 |
| Other | | | |
| Actinobacteria: Coriobacteriia: Coriobacteriales: | 1.1 | 0 | 0 |
| Coriobacteriaceae: none | | | |
| Bacteroidetes: [Saprospirae]: [Saprospirales]: | 1.1 | 0 | 0 |
| Saprospiraceae: none | | | |
| Bacteroidetes: Bacteroidia: Bacteroidales: | 1.1 | 0 | 0 |
| Porphyromonadaceae: Porphyromonas | | | |
| Bacteroidetes: Bacteroidia: Bacteroidales: | 1.1 | 0 | 0 |
| Porphyromonadaceae: Tannerella | | | |
| Bacteroidetes: Cytophagia: Cytophagales: | 1.1 | 0 | 0 |
| Cytophagaceae: Pontibacter | | | |
| Bacteroidetes: Cytophagia: Cytophagales: | 1.1 | 0 | 0 |
| Cytophagaceae: Spirosoma | | | |
| Bacteroidetes: Cytophagia: Cytophagales: | 1.1 | 0 | 0 |
| Flammeovirgaceae: Persicobacter | | | |
| Bacteroidetes: Flavobacteriia: Flavobacteriales: | 1.1 | 0 | 0 |
| Cryomorphaceae: Fluviicola | | | |
| Bacteroidetes: Flavobacteriia: Flavobacteriales: | 1.1 | 0 | 0 |
| Flavobacteriaceae: Aquimarina | | | |
| Bacteroidetes: Sphingobacteriia: Sphingobacteriales: | 1.1 | 0 | 0 |
| Sphingobacteriaceae: Pedobacter | | | |
| Chlorobi: BSV26: A89: none: none | 1.1 | 0 | 0 |
| Chloroflexi: Anaerolineae: envOPS12: none: none | 1.1 | 0 | 0 |
| Chloroflexi: Dehalococcoidetes: Dehalococcoidales: | 1.1 | 0 | 0 |
| none: none | | | |
| Chloroflexi: Dehalococcoidetes: none: none: none | 1.1 | 0 | 0 |
| Chloroflexi: Thermomicrobia: JG30-KF-CM45: none: | 1.1 | 0 | 0 |
| none | | | |
| Cyanobacteria: Nostocophycideae: Nostocales: | 1.1 | 0 | 0 |
| Nostocaceae: Nostoc | | | |
| Firmicutes: Bacilli: Bacillales: Bacillaceae: Other | 1.1 | 0 | 0 |
| Firmicutes: Bacilli: Bacillales: Paenibacillaceae: | 1.1 | 0 | 0 |
| Aneurinibacillus | | | |
| Firmicutes: Bacilli: Bacillales: Paenibacillaceae: | 1.1 | 0 | 0 |
| Brevibacillus | | | |
| Firmicutes: Bacilli: Lactobacillales: Carnobacteriaceae: | 1.1 | 0 | 0 |
| Carnobacterium | | | |
| Firmicutes: Bacilli: Lactobacillales: Streptococcaceae: | 1.1 | 0 | 0 |
| Other | | | |
| Firmicutes: Clostridia: Clostridiales: [Tissierellaceae]: | 1.1 | 0 | 0 |
| Parvimonas | | | |
| Firmicutes: Clostridia: Clostridiales: Eubacteriaceae: | 1.1 | 0 | 0 |
| Anaerofustis | | | |
| Firmicutes: Clostridia: Clostridiales: Lachnospiraceae: | 1.1 | 0 | 0.0001 |
| Butyrivibrio | | | |
| Firmicutes: Clostridia: Clostridiales: | 1.1 | 0 | 0 |
| Symbiobacteriaceae: Symbiobacterium | | | |
| Firmicutes: Clostridia: Clostridiales: Veillonellaceae: | 1.1 | 0 | 0 |
| none | | | |
| Firmicutes: Clostridia: Clostridiales: Veillonellaceae: | 1.1 | 0 | 0 |
| Selenomonas | | | |
| Firmicutes: Clostridia: Clostridiales: Veillonellaceae: | 1.1 | 0 | 0.0001 |
| Sporomusa | | | |
| Firmicutes: Other: Other: Other: Other | 1.1 | 0 | 0 |
| Fusobacteria: Fusobacteriia: Fusobacteriales: | 1.1 | 0 | 0 |
| Fusobacteriaceae: Propionigenium | | | |
| Nitrospirae: Nitrospira: Nitrospirales: | 1.1 | 0 | 0 |
| [Thermodesulfovibrionaceae]: LCP-6 | | | |
| OP9: JS1: SB-45: none: none | 1.1 | 0 | 0 |
| Planctomycetes: ODP123: T8-B82: none: none | 1.1 | 0 | 0 |
| Planctomycetes: Planctomycetia: Gemmatales: | 1.1 | 0 | 0 |
| Isosphaeraceae: none | | | |
| Planctomycetes: Planctomycetia: Planctomycetales: | 1.1 | 0 | 0.0001 |
| Planctomycetaceae: Planctomyces | | | |
| Proteobacteria: Alphaproteobacteria: Caulobacterales: | 1.1 | 0 | 0 |
| Caulobacteraceae: Brevundimonas | | | |
| Proteobacteria: Alphaproteobacteria: none: none: none | 1.1 | 0 | 0 |
| Proteobacteria: Alphaproteobacteria: RF32: none: none | 1.1 | 0 | 0 |
| Proteobacteria: Alphaproteobacteria: Rhizobiales: | 1.1 | 0 | 0 |
| Beijerinckiaceae: none | | | |
| Proteobacteria: Alphaproteobacteria: Rhizobiales: | 1.1 | 0 | 0 |
| Hyphomicrobiaceae: Hyphomicrobium | | | |
| Proteobacteria: Alphaproteobacteria: Rhizobiales: | 1.1 | 0 | 0 |
| Hyphomicrobiaceae: Rhodoplanes | | | |
| Proteobacteria: Alphaproteobacteria: Rhizobiales: | 1.1 | 0 | 0 |
| Methylobacteriaceae: Methylobacterium | | | |
| Proteobacteria: Alphaproteobacteria: Rhizobiales: none: | 1.1 | 0 | 0 |
| none | | | |
| Proteobacteria: Alphaproteobacteria: Rhodobacterales: | 1.1 | 0 | 0 |
| Rhodobacteraceae: Amaricoccus | | | |
| Proteobacteria: Alphaproteobacteria: Rhodobacterales: | 1.1 | 0 | 0 |
| Rhodobacteraceae: Octadecabacter | | | |
| Proteobacteria: Alphaproteobacteria: Rhodobacterales: | 1.1 | 0 | 0 |
| Rhodobacteraceae: Rhodobacter | | | |
| Proteobacteria: Betaproteobacteria: Burkholderiales: | 1.1 | 0 | 0 |
| Alcaligenaceae: none | | | |
| Proteobacteria: Betaproteobacteria: Burkholderiales: | 1.1 | 0 | 0 |
| Burkholderiaceae: Burkholderia | | | |
| Proteobacteria: Betaproteobacteria: Burkholderiales: | 1.1 | 0 | 0 |
| Burkholderiaceae: none | | | |
| Proteobacteria: Betaproteobacteria: Burkholderiales: | 1.1 | 0 | 0 |
| Comamonadaceae: Comamonas | | | |
| Proteobacteria: Betaproteobacteria: Burkholderiales: | 1.1 | 0 | 0 |
| Comamonadaceae: none | | | |
| Proteobacteria: Betaproteobacteria: Burkholderiales: | 1.1 | 0 | 0 |
| Comamonadaceae: Other | | | |
| Proteobacteria: Betaproteobacteria: Burkholderiales: | 1.1 | 0 | 0 |
| Oxalobacteraceae: Janthinobacterium | | | |
| Proteobacteria: Betaproteobacteria: Neisseriales: | 1.1 | 0 | 0.0001 |
| Neisseriaceae: Neisseria | | | |
| Proteobacteria: Betaproteobacteria: none: none: none | 1.1 | 0 | 0 |
| Proteobacteria: Betaproteobacteria: Rhodocyclales | 1.1 | 0 | 0 |
| Rhodocyclaceae: C39 | | | |
| Proteobacteria: Deltaproteobacteria: Desulfobacterales: | 1.1 | 0 | 0 |
| Desulfobacteraceae: none | | | |
| Proteobacteria: Deltaproteobacteria: Desulfovibrionales: | 1.1 | 0 | 0 |
| Desulfovibrionaceae: Desulfovibrio | | | |
| Proteobacteria: Deltaproteobacteria: | 1.1 | 0 | 0 |
| Desulfuromonadales: Geobacteraceae: Geobacter | | | |
| Proteobacteria: Deltaproteobacteria: GW-28: none: none | 1.1 | 0 | 0 |
| Proteobacteria: Deltaproteobacteria: Myxococcales: | 1.1 | 0 | 0 |
| none: none | | | |
| Proteobacteria: Deltaproteobacteria: none: none: none | 1.1 | 0 | 0 |
| Proteobacteria: Deltaproteobacteria: | 1.1 | 0 | 0 |
| Syntrophobacterales: Syntrophaceae: Desulfobacca | | | |
| Proteobacteria: Epsilonproteobacteria: | 1.1 | 0 | 0 |
| Campylobacterales: Helicobacteraceae: none | | | |
| Proteobacteria: Gammaproteobacteria: Aeromonadales: | 1.1 | 0 | 0.0003 |
| Succinivibrionaceae: Succinivibrio | | | |
| Proteobacteria: Gammaproteobacteria: Alteromonadales: | 1.1 | 0 | 0 |
| Colwelliaceae: none | | | |
| Proteobacteria: Gammaproteobacteria: Alteromonadales: | 1.1 | 0 | 0 |
| HTCC2188: HTCC | | | |
| Proteobacteria: Gammaproteobacteria: Alteromonadales: | 1.1 | 0 | 0 |
| none: none | | | |
| Proteobacteria: Gammaproteobacteria: Chromatiales: | 1.1 | 0 | 0 |
| none: none | | | |
| Proteobacteria: Gammaproteobacteria: Enterobacteriales: | 1.1 | 0 | 0 |
| Enterobacteriaceae: Erwinia | | | |
| Proteobacteria: Gammaproteobacteria: Enterobacteriales: | 1.1 | 0 | 0.0001 |
| Enterobacteriaceae: Proteus | | | |
| Proteobacteria: Gammaproteobacteria: Enterobacteriales: | 1.1 | 0 | 0 |
| Enterobacteriaceae: Providencia | | | |
| Proteobacteria: Gammaproteobacteria: Enterobacteriales: | 1.1 | 0 | 0 |
| Enterobacteriaceae: Serratia | | | |
| Proteobacteria: Gammaproteobacteria: Oceanospirillales: | 1.1 | 0 | 0 |
| Oleiphilaceae: none | | | |
| Proteobacteria: Gammaproteobacteria: Pasteurellales: | 1.1 | 0 | 0 |
| Pasteurellaceae: Haemophilus | | | |
| Proteobacteria: Gammaproteobacteria: Pasteurellales: | 1.1 | 0 | 0.0001 |
| Pasteurellaceae: Mannheimia | | | |
| Proteobacteria: Gammaproteobacteria: Pasteurellales: | 1.1 | 0 | 0 |
| Pasteurellaceae: none | | | |
| Proteobacteria: Gammaproteobacteria: | 1.1 | 0 | 0 |
| Pseudomonadales: Moraxellaceae: Acinetobacter | | | |
| Proteobacteria: Gammaproteobacteria: Vibrionales: | 1.1 | 0 | 0 |
| Pseudoalteromonadaceae: Pseudoalteromonas | | | |
| Proteobacteria: Gammaproteobacteria: Vibrionales: | 1.1 | 0 | 0 |
| Vibrionaceae: Other | | | |
| Proteobacteria: none: none: none: none | 1.1 | 0 | 0 |
| SC4: none: none: none: none | 1.1 | 0 | 0 |
| Spirochaetes: [Brachyspirae]: [Brachyspirales]: | 1.1 | 0 | 0 |
| Brachyspiraceae: Brachyspira | | | |
| Tenericutes: Mollicutes: RF39: none: none | 1.1 | 0 | 0 |
| Verrucomicrobia: Verrucomicrobiae: | 1.1 | 0 | 0 |
| Verrucomicrobiales: Verrucomicrobiaceae: Akkermansia | | | |

TABLE 5
Identified Microorganisms in Cats

| | % | | Highest |
|---|---|---|---|
| | ind'ls | Mean % | % in |
| Phylum: Class: Order: Family: Genus | in popn | in ind'l | ind'l |
| Bacteroidetes: Bacteroidia: Bacteroidales: Bacteroidaceae: | 100 | 0.1722 | 0.3291 |
| Bacteroides | | | |
| Firmicutes: Clostridia: Clostridiales: Lachnospiraceae: | 100 | 0.0734 | 0.1451 |
| Blautia | | | |
| Firmicutes: Clostridia: Clostridiales: Lachnospiraceae: none | 100 | 0.0531 | 0.1067 |
| Actinobacteria: Coriobacteriia: Coriobacteriales: | 100 | 0.0211 | 0.0595 |
| Coriobacteriaceae: Collinsella | | | |
| Firmicutes: Clostridia: Clostridiales: Lachnospiraceae: Dorea | 100 | 0.0152 | 0.0377 |
| Firmicutes: Clostridia: Clostridiales: Lachnospiraceae: | 100 | 0.0123 | 0.028 |
| [Ruminococcus] | | | |
| Firmicutes: Clostridia: Clostridiales: none: none | 98.8 | 0.0255 | 0.0524 |
| Firmicutes: Clostridia: Clostridiales: Clostridiaceae: none | 96.4 | 0.0406 | 0.096 |
| Proteobacteria: Betaproteobacteria: Burkholderiales: | 96.4 | 0.0243 | 0.0517 |
| Alcaligenaceae: Sutterella | | | |
| Firmicutes: Clostridia: Clostridiales: Ruminococcaceae: | 96.4 | 0.019 | 0.0385 |
| none | | | |
| Firmicutes: Clostridia: Clostridiales: Clostridiaceae: | 96.4 | 0.0033 | 0.0085 |
| Clostridium | | | |
| Bacteroidetes: Bacteroidia: Bacteroidales: Prevotellaceae: | 92.9 | 0.2615 | 0.538 |
| Prevotella | | | |
| Firmicutes: Erysipelotrichi: Erysipelotrichales: | 92.9 | 0.0187 | 0.0633 |
| Erysipelotrichaceae: [Eubacterium] | | | |
| Firmicutes: Clostridia: Clostridiales: Ruminococcaceae: | 91.7 | 0.0097 | 0.021 |
| Oscillospira | | | |
| Firmicutes: Clostridia: Clostridiales: Clostridiaceae: Other | 91.7 | 0.0059 | 0.021 |
| Firmicutes: Clostridia: Clostridiales: Lachnospiraceae: Other | 91.7 | 0.0048 | 0.0115 |
| Firmicutes: Clostridia: Clostridiales: Ruminococcaceae: | 88.1 | 0.01 | 0.0288 |
| Ruminococcus | | | |
| Firmicutes: Clostridia: Clostridiales: Lachnospiraceae: | 88.1 | 0.003 | 0.0079 |
| Coprococcus | | | |
| Bacteroidetes: Bacteroidia: Bacteroidales: | 86.9 | 0.0076 | 0.0216 |
| Porphyromonadaceae: Parabacteroides | | | |
| Fusobacteria: Fusobacteriia: Fusobacteriales: | 85.7 | 0.0647 | 0.1535 |
| Fusobacteriaceae: Fusobacterium | | | |
| Firmicutes: Clostridia: Clostridiales: Ruminococcaceae: | 82.1 | 0.0122 | 0.032 |
| Faecalibacterium | | | |
| Firmicutes: Clostridia: Clostridiales: Veillonellaceae: | 81 | 0.0254 | 0.072 |
| Megamonas | | | |
| Firmicutes: Clostridia: Clostridiales: Lachnospiraceae: | 70.2 | 0.0016 | 0.0058 |
| Roseburia | | | |
| Other: Other: Other: Other: Other | 66.7 | 0.0006 | 0.0016 |
| Firmicutes: Erysipelotrichi: Erysipelotrichales: | 63.1 | 0.0043 | 0.0145 |
| Erysipelotrichaceae: none | | | |
| Firmicutes: Clostridia: Clostridiales: Other: Other | 60.7 | 0.0008 | 0.0023 |
| Firmicutes: Clostridia: Clostridiales: [Mogibacteriaceae]: | 56 | 0.0009 | 0.0027 |
| none | | | |
| Firmicutes: Erysipelotrichi: Erysipelotrichales: | 54.8 | 0.0164 | 0.0529 |
| Erysipelotrichaceae: Catenibacterium | | | |
| Firmicutes: Clostridia: Clostridiales: Veillonellaceae: | 54.8 | 0.006 | 0.0145 |
| Phascolarctobacterium | | | |
| Firmicutes: Clostridia: Clostridiales: Peptococcaceae: | 52.4 | 0.0017 | 0.0044 |
| Peptococcus | | | |
| Firmicutes: Clostridia: Clostridiales: Veillonellaceae: | 51.2 | 0.0048 | 0.0231 |
| Megasphaera | | | |
| Firmicutes: Bacilli: Lactobacillales: Enterococcaceae: | 50 | 0.0021 | 0.0096 |
| Enterococcus | | | |
| Proteobacteria: Gammaproteobacteria: Enterobacteriales: | 48.8 | 0.0018 | 0.0071 |
| Enterobacteriaceae: none | | | |
| Actinobacteria: Coriobacteriia: Coriobacteriales: | 47.6 | 0.0015 | 0.0059 |
| Coriobacteriaceae: none | | | |
| Firmicutes: Clostridia: Clostridiales: Ruminococcaceae: | 47.6 | 0.0004 | 0.0013 |
| Other | | | |
| Actinobacteria: Actinobacteria: Bifidobacteriales: | 46.4 | 0.005 | 0.022 |
| Bifidobacteriaceae: Bifidobacterium | | | |
| Actinobacteria: Coriobacteriia: Coriobacteriales: | 40.5 | 0.0002 | 0.0005 |
| Coriobacteriaceae: Slackia | | | |
| Firmicutes: Clostridia: Clostridiales: Peptostreptococcaceae: | 39.3 | 0.0027 | 0.0097 |
| none | | | |
| Bacteroidetes: Bacteroidia: Bacteroidales: | 38.1 | 0.0115 | 0.0412 |
| [Paraprevotellaceae]: [Prevotella] | | | |
| Bacteroidetes: Bacteroidia: Bacteroidales: | 36.9 | 0.0062 | 0.03 |
| [Odoribacteraceae]: Odoribacter | | | |
| Firmicutes: Clostridia: Clostridiales: Veillonellaceae: | 36.9 | 0.0039 | 0.0111 |
| Dialister | | | |
| Firmicutes: Bacilli: Lactobacillales: Lactobacillaceae: | 35.7 | 0.0171 | 0.1081 |
| Lactobacillus | | | |
| Proteobacteria: Gammaproteobacteria: Aeromonadales: | 33.3 | 0.0019 | 0.0069 |
| Succinivibrionaceae: Anaerobiospirillum | | | |
| Firmicutes: Bacilli: Lactobacillales: Streptococcaceae: | 29.8 | 0.0005 | 0.0022 |
| Streptococcus | | | |
| Firmicutes: Clostridia: Clostridiales: Lachnospiraceae: | 28.6 | 0.0008 | 0.0042 |
| Lachnospira | | | |
| Proteobacteria: Gammaproteobacteria: Aeromonadales: | 28.6 | 0.0004 | 0.0016 |
| Succinivibrionaceae: none | | | |
| Proteobacteria: Deltaproteobacteria: Desulfovibrionales: | 27.4 | 0.0007 | 0.0023 |
| Desulfovibrionaceae: Desulfovibrio | | | |
| Proteobacteria: Epsilonproteobacteria: Campylobacterales: | 26.2 | 0.0001 | 0.0004 |
| Campylobacteraceae: Campylobacter | | | |
| Actinobacteria: Coriobacteriia: Coriobacteriales: | 23.8 | 0.0001 | 0.0003 |
| Coriobacteriaceae: Adlercreutzia | | | |
| Firmicutes: Clostridia: Clostridiales: Peptostreptococcaceae: | 22.6 | 0.0006 | 0.0048 |
| Peptostreptococcus | | | |
| Bacteroidetes: Bacteroidia: Bacteroidales: Rikenellaceae: | 21.4 | 0.0004 | 0.0024 |
| Other | | | |
| Proteobacteria: Deltaproteobacteria: Desulfovibrionales: | 21.4 | 0.0003 | 0.0018 |
| Desulfovibrionaceae: none | | | |
| Firmicutes: Bacilli: Turicibacterales: Turicibacteraceae: | 20.2 | 0.0022 | 0.0174 |
| Turicibacter | | | |
| Bacteroidetes: Bacteroidia: Bacteroidales: [Barnesiellaceae]: | 20.2 | 0.0006 | 0.003 |
| none | | | |
| Firmicutes: Clostridia: Clostridiales: Peptostreptococcaceae: | 20.2 | 0.0006 | 0.0054 |
| Other | | | |
| Proteobacteria: Epsilonproteobacteria: Campylobacterales: | 17.9 | 0.0001 | 0.0004 |
| Helicobacteraceae: Helicobacter | | | |
| Bacteroidetes: Bacteroidia: Bacteroidales: | 16.7 | 0.0057 | 0.0567 |
| [Paraprevotellaceae]: Paraprevotella | | | |
| Firmicutes: Clostridia: Clostridiales: Veillonellaceae: | 16.7 | 0.0011 | 0.0049 |
| Acidaminococcus | | | |
| Actinobacteria: Actinobacteria: Actinomycetales: | 16.7 | 0.0001 | 0.0005 |
| Actinomycetaceae: Actinomyces | | | |
| Bacteroidetes: Bacteroidia: Bacteroidales: S24-7: none | 15.5 | 0.0011 | 0.0081 |
| Firmicutes: Erysipelotrichi: Erysipelotrichales: | 15.5 | 0.0001 | 0.0003 |
| Erysipelotrichaceae: Holdemania | | | |
| Bacteroidetes: Bacteroidia: Bacteroidales: Rikenellaceae: | 14.3 | 0.0003 | 0.0018 |
| none | | | |
| Proteobacteria: Gammaproteobacteria: Aeromonadales: | 13.1 | 0.0038 | 0.021 |
| Succinivibrionaceae: Succinivibrio | | | |
| Firmicutes: Bacilli: Lactobacillales: Streptococcaceae: | 13.1 | 0.0008 | 0.0042 |
| Lactococcus | | | |
| Firmicutes: Erysipelotrichi: Erysipelotrichales: | 11.9 | 0.0003 | 0.002 |
| Erysipelotrichaceae: Coprobacillus | | | |
| Tenericutes: Mollicutes: RF39: none: none | 10.7 | 0.0007 | 0.0047 |
| Proteobacteria: Betaproteobacteria: Burkholderiales: Other: | 10.7 | 0.0002 | 0.001 |
| Other | | | |
| Firmicutes: Erysipelotrichi: Erysipelotrichales: | 9.5 | 0.0002 | 0.0015 |
| Erysipelotrichaceae: Allobaculum | | | |
| Firmicutes: Clostridia: Clostridiales: [Mogibacteriaceae]: | 9.5 | 0.0001 | 0.0004 |
| Other | | | |
| Firmicutes: Clostridia: Clostridiales: Christensenellaceae: | 9.5 | 0.0001 | 0.0003 |
| none | | | |
| Firmicutes: Clostridia: Clostridiales: Clostridiaceae: | 9.5 | 0.0001 | 0.0006 |
| Candidatus Arthromitus | | | |
| Firmicutes: Clostridia: Clostridiales: Lachnospiraceae: | 9.5 | 0.0001 | 0.001 |
| Epulopiscium | | | |
| Firmicutes: Clostridia: Clostridiales: Veillonellaceae: | 9.5 | 0.0001 | 0.0003 |
| Veillonella | | | |
| Firmicutes: Bacilli: Lactobacillales: Enterococcaceae: Other | 8.3 | 0.0005 | 0.0027 |
| Bacteroidetes: Bacteroidia: Bacteroidales: | 7.1 | 0.0007 | 0.0054 |
| [Odoribacteraceae]: Butyricimonas | | | |
| Proteobacteria: Deltaproteobacteria: Desulfovibrionales: | 7.1 | 0.0002 | 0.0013 |
| Desulfovibrionaceae: Bilophila | | | |
| Firmicutes: Clostridia: Clostridiales: Clostridiaceae: Sarcina | 7.1 | 0.0001 | 0.0004 |
| Firmicutes: Clostridia: Clostridiales: Lachnospiraceae: | 7.1 | 0.0001 | 0.0004 |
| Anaerostipes | | | |
| Proteobacteria: Gammaproteobacteria: Alteromonadales: | 7.1 | 0.0001 | 0.0003 |
| Shewanellaceae: Shewanella | | | |
| Bacteroidetes: Bacteroidia: Bacteroidales: Bacteroidaceae: | 7.1 | 0 | 0.0001 |
| 5-7N15 | | | |
| Firmicutes: Bacilli: Lactobacillales: none: none | 6 | 0.0001 | 0.0008 |
| Proteobacteria: Gammaproteobacteria: Oceanospirillales: | 6 | 0.0001 | 0.0006 |
| Halomonadaceae: Halomonas | | | |
| Firmicutes: Clostridia: Clostridiales: Clostridiaceae: SMB53 | 6 | 0 | 0.0001 |
| Firmicutes: Clostridia: Clostridiales: Eubacteriaceae: | 6 | 0 | 0.0001 |
| Anaerofustis | | | |
| Fusobacteria: Fusobacteriia: Fusobacteriales: | 6 | 0 | 0.0002 |
| Fusobacteriaceae: Cetobacterium | | | |
| Firmicutes: Clostridia: Clostridiales: Veillonellaceae: none | 4.8 | 0.0004 | 0.0034 |
| Bacteroidetes: Bacteroidia: Bacteroidales: Other: Other | 4.8 | 0 | 0.0002 |
| Firmicutes: Bacilli: Bacillales: Staphylococcaceae: | 4.8 | 0 | 0.0001 |
| Staphylococcus | | | |
| Firmicutes: Bacilli: Lactobacillales: Other: Other | 4.8 | 0 | 0.0001 |
| Proteobacteria: Betaproteobacteria: Burkholderiales: | 4.8 | 0 | 0.0001 |
| Comamonadaceae: none | | | |
| Bacteroidetes: Bacteroidia: Bacteroidales: | 3.6 | 0 | 0 |
| Porphyromonadaceae: Porphyromonas | | | |
| Firmicutes: Clostridia: Clostridiales: [Tissierellaceae]: | 3.6 | 0 | 0 |
| Parvimonas | | | |
| Firmicutes: Clostridia: Clostridiales: Lachnospiraceae: | 3.6 | 0 | 0.0002 |
| Clostridium | | | |
| Firmicutes: Clostridia: Clostridiales: Veillonellaceae: Other | 3.6 | 0 | 0 |
| OP9: JS1: SB-45: none: none | 3.6 | 0 | 0.0001 |
| Proteobacteria: Betaproteobacteria: Neisseriales: | 3.6 | 0 | 0.0003 |
| Neisseriaceae: none | | | |
| Proteobacteria: Gammaproteobacteria: Pasteurellales: | 3.6 | 0 | 0.0001 |
| Pasteurellaceae: none | | | |
| Proteobacteria: Alphaproteobacteria: RF32: none: none | 2.4 | 0.0002 | 0.002 |
| Firmicutes: Bacilli: Lactobacillales: Leuconostocaceae: | 2.4 | 0.0001 | 0.0011 |
| Leuconostoc | | | |
| Acidobacteria: Acidobacteria-6: iii1-15: mb2424: none | 2.4 | 0 | 0 |
| Actinobacteria: Actinobacteria: Actinomycetales: | 2.4 | 0 | 0 |
| Micrococcaceae: none | | | |
| Actinobacteria: Actinobacteria: Actinomycetales: | 2.4 | 0 | 0.0001 |
| Yaniellaceae: Yaniella | | | |
| Actinobacteria: Coriobacteriia: Coriobacteriales: | 2.4 | 0 | 0.0001 |
| Coriobacteriaceae: Eggerthella | | | |
| Bacteroidetes: [Saprospirae]: [Saprospirales]: | 2.4 | 0 | 0 |
| Chitinophagaceae: Flavisolibacter | | | |
| Firmicutes: Bacilli: Lactobacillales: Lactobacillaceae: Other | 2.4 | 0 | 0.0001 |
| Firmicutes: Clostridia: Clostridiales: [Mogibacteriaceae]: | 2.4 | 0 | 0.0001 |
| Mogibacterium | | | |
| Proteobacteria: Alphaproteobacteria: Rhodobacterales: | 2.4 | 0 | 0 |
| Rhodobacteraceae: Octadecabacter | | | |
| Proteobacteria: Betaproteobacteria: Burkholderiales: | 2.4 | 0 | 0 |
| Burkholderiaceae: Lautropia | | | |
| Proteobacteria: Betaproteobacteria: Burkholderiales: | 2.4 | 0 | 0 |
| Oxalobacteraceae: none | | | |
| Proteobacteria: Gammaproteobacteria: Oceanospirillales: | 2.4 | 0 | 0 |
| Oceanospirillaceae: none | | | |
| Verrucomicrobia: [Spartobacteria]: [Chthoniobacterales]: | 2.4 | 0 | 0 |
| [Chthoniobacteraceae]: Chthoniobacter | | | |
| Firmicutes: Clostridia: Clostridiales: [Tissierellaceae]: | 1.2 | 0.0001 | 0.0006 |
| Peptoniphilus | | | |
| Actinobacteria: Actinobacteria: Actinomycetales: | 1.2 | 0 | 0 |
| Brevibacteriaceae: Brevibacterium | | | |
| Actinobacteria: Actinobacteria: Actinomycetales: | 1.2 | 0 | 0 |
| Corynebacteriaceae: Corynebacterium | | | |
| Actinobacteria: Actinobacteria: Actinomycetales: | 1.2 | 0 | 0 |
| Micrococcaceae: Micrococcus | | | |
| Bacteroidetes: [Rhodothermi]: [Rhodothermales]: | 1.2 | 0 | 0 |
| [Balneolaceae]: Balneola | | | |
| Bacteroidetes: Bacteroidia: Bacteroidales: none: none | 1.2 | 0 | 0 |
| Bacteroidetes: Cytophagia: Cytophagales: Cytophagaceae: | 1.2 | 0 | 0 |
| Hymenobacter | | | |
| Bacteroidetes: Flavobacteriia: Flavobacteriales: | 1.2 | 0 | 0 |
| [Weeksellaceae]: Chryseobacterium | | | |
| Bacteroidetes: Flavobacteriia: Flavobacteriales: | 1.2 | 0 | 0 |
| Flavobacteriaceae: none | | | |
| Bacteroidetes: Flavobacteriia: Flavobacteriales: | 1.2 | 0 | 0 |
| Flavobacteriaceae: Polaribacter | | | |
| Chlamydiae: Chlamydiia: Chlamydiales: Chlamydiaceae: | 1.2 | 0 | 0 |
| Other | | | |
| Chloroflexi: Anaerolineae: GCA004: none: none | 1.2 | 0 | 0 |
| Cyanobacteria: 4C0d-2: YS2: none: none | 1.2 | 0 | 0.0001 |
| Cyanobacteria: Nostocophycideae: Nostocales: Nostocaceae: | 1.2 | 0 | 0 |
| none | | | |
| Cyanobacteria: Oscillatoriophycideae: Chroococcales: | 1.2 | 0 | 0 |
| Xenococcaceae: Chroococcidiopsis | | | |
| Euryarchaeota: Thermoplasmata: E2: DHVEG-1: none | 1.2 | 0 | 0 |
| Euryarchaeota: Thermoplasmata: E2: Marine group II: none | 1.2 | 0 | 0 |
| Firmicutes: Bacilli: Bacillales: Paenibacillaceae: | 1.2 | 0 | 0 |
| Paenibacillus | | | |
| Firmicutes: Bacilli: Bacillales: Planococcaceae: none | 1.2 | 0 | 0 |
| Firmicutes: Bacilli: Bacillales: Planococcaceae: Sporosarcina | 1.2 | 0 | 0 |
| Firmicutes: Bacilli: Lactobacillales: Aerococcaceae: | 1.2 | 0 | 0 |
| Facklamia | | | |
| Firmicutes: Bacilli: Lactobacillales: Lactobacillaceae: | 1.2 | 0 | 0 |
| Pediococcus | | | |
| Firmicutes: Bacilli: Lactobacillales: Leuconostocaceae: none | 1.2 | 0 | 0 |
| Firmicutes: Clostridia: Clostridiales: [Tissierellaceae]: ph2 | 1.2 | 0 | 0 |
| Firmicutes: Clostridia: Clostridiales: Christensenellaceae: | 1.2 | 0 | 0 |
| Christensenella | | | |
| Firmicutes: Clostridia: Clostridiales: Clostridiaceae: 02d06 | 1.2 | 0 | 0 |
| Firmicutes: Clostridia: Clostridiales: EtOH8: none | 1.2 | 0 | 0.0001 |
| Firmicutes: Clostridia: Clostridiales: Eubacteriaceae: | 1.2 | 0 | 0 |
| Pseudoramibacter_Eubacterium | | | |
| Firmicutes: Clostridia: Clostridiales: Peptococcaceae: none | 1.2 | 0 | 0 |
| Firmicutes: Clostridia: Clostridiales: Peptococcaceae: Other | 1.2 | 0 | 0 |
| Firmicutes: Clostridia: Clostridiales: Peptococcaceae: rc4-4 | 1.2 | 0 | 0 |
| Firmicutes: Clostridia: Clostridiales: Veillonellaceae: | 1.2 | 0 | 0.0001 |
| Succiniclasticum | | | |
| Firmicutes: Erysipelotrichi: Erysipelotrichales: | 1.2 | 0 | 0 |
| Erysipelotrichaceae: Bulleidia | | | |
| Firmicutes: Erysipelotrichi: Erysipelotrichales: | 1.2 | 0 | 0 |
| Erysipelotrichaceae: Clostridium | | | |
| Firmicutes: Erysipelotrichi: Erysipelotrichales: | 1.2 | 0 | 0.0001 |
| Erysipelotrichaceae: Sharpea | | | |
| Fusobacteria: Fusobacteriia: Fusobacteriales: | 1.2 | 0 | 0 |
| Fusobacteriaceae: Other | | | |
| Lentisphaerae: [Lentisphaeria]: Victivallales: Victivallaceae: | 1.2 | 0 | 0 |
| none | | | |
| Other: Other: Other: Other: Other | 1.2 | 0 | 0 |
| Planctomycetes: Phycisphaerae: WD2101: none: none | 1.2 | 0 | 0 |
| Planctomycetes: Planctomycetia: Gemmatales: | 1.2 | 0 | 0 |
| Isosphaeraceae: none | | | |
| Proteobacteria: Betaproteobacteria: Burkholderiales: | 1.2 | 0 | 0 |
| Oxalobacteraceae: Janthinobacterium | | | |
| Proteobacteria: Betaproteobacteria: Ellin6067: none: none | 1.2 | 0 | 0 |
| Proteobacteria: Gammaproteobacteria: Enterobacteriales: | 1.2 | 0 | 0 |
| Enterobacteriaceae: Citrobacter | | | |
| Proteobacteria: Gammaproteobacteria: Enterobacteriales: | 1.2 | 0 | 0 |
| Enterobacteriaceae: Erwinia | | | |
| Proteobacteria: Gammaproteobacteria: Enterobacteriales: | 1.2 | 0 | 0.0001 |
| Enterobacteriaceae: Other | | | |
| Proteobacteria: Gammaproteobacteria: Enterobacteriales: | 1.2 | 0 | 0 |
| Enterobacteriaceae: Proteus | | | |
| Proteobacteria: Gammaproteobacteria: Oceanospirillales: | 1.2 | 0 | 0 |
| Other: Other | | | |
| Proteobacteria: Gammaproteobacteria: Pasteurellales: | 1.2 | 0 | 0 |
| Pasteurellaceae: Actinobacillus | | | |
| Proteobacteria: Gammaproteobacteria: Pasteurellales: | 1.2 | 0 | 0.0001 |
| Pasteurellaceae: Aggregatibacter | | | |
| Proteobacteria: Gammaproteobacteria: Pasteurellales: | 1.2 | 0 | 0 |
| Pasteurellaceae: Pasteurella | | | |
| Proteobacteria: Gammaproteobacteria: Pseudomonadales: | 1.2 | 0 | 0 |
| Pseudomonadaceae: Pseudomonas | | | |
| Proteobacteria: Gammaproteobacteria: Vibrionales: | 1.2 | 0 | 0 |
| Pseudoalteromonadaceae: none | | | |
| Proteobacteria: Gammaproteobacteria: Vibrionales: | 1.2 | 0 | 0 |
| Vibrionaceae: none | | | |
| Proteobacteria: Gammaproteobacteria: Vibrionales: | 1.2 | 0 | 0 |
| Vibrionaceae: Vibrio | | | |
| VHS-B3-43: none: none: none: none | 1.2 | 0 | 0 |

### Example 7—Case Study for Hemorrhagic Gastroenteritis

A 16 year old McNab Shepherd (Canis lupus familiaris) began exhibiting clinical signs associated with hemorrhagic gastroenteritis (HGE), including bloody stools and vomiting when she was 14 years old. The first time this happened, she was hospitalized and received IV fluids and antibiotics overnight. Her HGE was responsive to metronidazole in combination with a bland diet of boiled chicken and white rice. But after this incident, she developed many food sensitivities and had bouts of bloody diarrhea every few weeks. When she didn't have diarrhea, she was often constipated and her body condition declined. After nearly two years of HGE flare-ups, she took a course of 50 oral FMT capsules PO×QD for four days and BD for 23 days, given with food, starting just after completing another course of metronidazole. During the course of the oral FMT treatment her fecal consistency improved, fecal color shifted from yellow to brown, and she was eventually able to tolerate many more protein sources. It has now been nearly 12 months since she received the oral fecal transplant and she hasn't exhibited any further signs of HGE.

### Example 8—Case Study for Inflammatory Bowel Disease

A Boxer mix (Canis lupus familiaris) was healthy until she turned 5 years old. Over the course of just a few months, she developed severe diarrhea and vomiting. Various treatments tried included antibiotics, antacids, probiotics, and prescription diets, but none of these treatments seemed to alleviate her symptoms and she continued to worsen. After an official Inflammatory Bowel Disease (IBD) diagnosis, she began a high daily dose of Prednisone in addition to her other medications. Her diarrhea temporarily resolved, but after lowering the Prednisone dosage to minimize side effects, her IBD relapsed. She once again developed watery, diarrhea, and it was not alleviated even after significantly increasing the Prednisone dosage, instead of resolving her digestive issues, the prednisone increase prompted an onset of medication-induced Cushing's disease, turning this once energetic, muscular dog into a frail, low-energy one. Despite the steroid, her digestive issues persisted. Finally, she was treated with the oral FMT capsules, which she took three times daily over the course of several months PO TID×three months, given with food. Slowly her fecal consistency changed from a yellowish liquid to a healthy brown solid, and she has been tapered off all prescription medications.

### Example 9—Case Study for Tritrichomonas foetus

Two Domestic Shorthair cats (Felis catus), one female and one male from the same litter, presented with a history of chronic bloody diarrhea beginning at eight weeks of age. Symptoms were unresponsive to prescription diets and probiotic supplements, and repeated fecal PCR tests were negative for all pathogens. Polymerase Chain Reactions (PCRs) use DNA primers to identify and amplify specific segments of DNA that are associated with certain pathogens. Kittens were consistently bright, alert, and responsive and had no evidence of other health issues. Repeated fecal PCRs testing for Giardia, a protozoan that also causes diarrhea were also negative. At 8 months of age, both cats tested positive for Tritrichomonas foetus, a parasite commonly found in cats that causes diarrhea, and began a 14-day course of Ronidazole shortly thereafter. Symptoms were not resolved with the course of Ronidazole, and two weeks after its conclusion, both cats began fecal microbiota transplant (FMT) capsules PO BID×25 days, administered with food. By the conclusion of the FMT capsules, one kitten's diarrhea had ceased completely, and the other had only intermittent diarrhea that was considerably less severe than previously.

### Example 10—Case Study for Atopic Dermatitis

A 6-year-old female Shiba Inu (Canis lupus familiaris) who was surrendered to a city shelter presented with severe atopic dermatitis that was unresponsive to prescription diets, antibiotics, steroids, and oclacitinib. Her skin was severely inflamed and was bright red over the vast majority of her body. She retained only a few patches of fur, and they were brittle and coarse. The dog was constantly uncomfortable, agitated, and itchy; she was unpredictable and would snap frequently at people who approached her. After completing a course of 50 FMT capsules PO×BID for 25 days, administered with food, the dog has fur covering most of her body, and it is noticeably healthier and softer. Her skin is no longer red and inflamed. Her temperament has improved considerably, and rescue staff reported that she began to show signs of affection and make attempts at play after just one week on the FMT capsules. She rarely scratches and appears to be much more comfortable overall.

### Example 11—Case Study for Campylobacter

A 4-year-old female Sheltie (Canis lupus familiaris) suffered from severe intermittent diarrhea, with flare-ups every few weeks, for approximately 2 years. Fecal PCRs consistently came back positive for Campylobacter. By 4 years old, she had been on a minimum of five rounds of Tylosin, which reduced the diarrhea in the short term, but were not effective in the long term, as the diarrhea would return and another PCR would reveal infection with campylobacter. Bouts of diarrhea caused the normally energetic young dog to be quiet and moderately depressed, with occasional vomiting and fecal incontinence. The dog was put on a round of 50 FMT capsules, PO×BID for 25 days, administered with food, which resolved the current episode of diarrhea within one week. In addition, the dog has been diarrhea-free for two months thus far, which is the longest diarrhea-free time period she has ever experienced.

### Example 12—Case Study for Clostridium perfringens

A 1-year-old female Miniature Schnauzer (Canis lupus familiaris) presented with chronic diarrhea and intermittent bouts of hemorrhagic gastroenteritis that required emergency hospital stays. A fecal PCR came back positive for Clostridium perfringens, which was treated with a 30-day course of Tylosin. Diarrhea persisted throughout the course of antibiotics and after its conclusion. Shortly after finishing the round of Tylosin, the dog began a 25-day course of FMT capsules, PO×BID for 25 days, given with food. Within 4 weeks, the dog's diarrhea had resolved completely, and it has not returned to date (6 months thus far).

### Example 13—Case Study for Parvovirus

A 14-week-old female German Shepherd/Husky mixed breed (Canis lupus familiaris) puppy received a DAPP vaccine at 8 weeks, but was not given a booster vaccine at 12 weeks. At 14 weeks, she developed classic signs of parvovirus infection (parvo): severe lethargy, dehydration, and uncontrollable bloody diarrhea. The puppy was admitted to the hospital and was started on supportive care, including IV fluids and antibiotics. Despite early treatment and close monitoring, the puppy continued to become more lethargic, and her white blood cell count continued to drop. By the fifth day of supportive care, she had lost 20% of her body weight. On the sixth day of hospitalization, the dog began treatment with 50 FMT capsules, PO TID, administered with food, for 17 days. No other aspects of treatment were modified at this time. Eighteen (18) hours after the first dose of the FMT capsule, the owner and the hospital staff noticed that the puppy's energy levels were increasing, and she began eating on her own. Within 36 hours, she was considered safe to be discharged from the hospital. Ultimately the puppy made a full recovery, and completed a full course of treatment with the FMT capsules.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A solid composition for oral administration comprising:
(a) microorganisms of at least one of the following taxa:
Ruminoccoccus, at least one member of the family Clostridiaceae, at least one member of the family Lachnospiraceae that is not Dorea, Blautia, or Fusobacterium, at least one member of the order Clostridiales, Bacteroides, Sutterella, Eubacterium, Collinsella, Megamonas, at least one member of the family Ruminococcaceae, Clostridium, Prevotella, at least another member of the family Clostridiaceae, at least another member of the family Lachnospiraceae that is not Blautia and is not Dorea, Faecalibacterium;
and
(b) microorganisms of at least one of the following taxa, which may be the same as or different from the microorganisms of the at least one taxa in (a): at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, Megamonas;
and/or
microorganisms of at least one of the following taxa, which may be the same as or different from the microorganisms of the at least one taxa in (a): Dorea, at least one member of the order Clostridiales, Sutterella, Eubacterium, Collinsella, at least one member of the family Erysipelotrichaceae, Megamonas, at least one member of the family Ruminococcaceae, Clostridium, at least another member of the family Clostridiaceae, at least another member of the family Lachnospiraceae that is not Blautia and that is not Dorea, Faecalibacterium.

2. The composition of claim 1, wherein the composition comprises at least four of the taxa in (a).

3. The composition of claim 1, wherein the composition comprises fiber.

4. (canceled)

5. The composition of claim 1, wherein the composition comprises fecal material from a non-human mammal.

6. (canceled)

7. The composition of claim 5, wherein the fecal material is feline fecal material, and wherein the composition comprises microorganisms of at least two of the following taxa: Dorea, Ruminoccoccus, Bacteroides, at least one member of the family Lachnospiraceae that is not Blautia and that is not Dorea, Collinsella, Blautia, at least one member of the order Clostridiales, at least one member of the family Ruminococcaceae, Clostridium, at least one member of the family Clostridiaceae, Sutterella, Prevotella, Eubacterium, Oscillospira, at least another member of the family Clostridiaceae, at least another member of the family Lachnospiraceae that is not Blautia and that is not Dorea, Ruminococcus, Coprococcus, Parabacteroides, Fusobacterium, Faecalibacterium, Megamonas.

8. (canceled)

9. (canceled)

10. The composition of claim 5, wherein the fecal material is canine fecal material, and wherein the composition comprises microorganisms of at least two of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, at least one member of the family Erysipelotrichaceae, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium.

11. (canceled)

12. The composition of claim 10, wherein the composition comprises microorganisms of at least three of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, at least one member of the family Erysipelotrichaceae, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium.

13. (canceled)

14. (canceled)

15. The composition of claim 1, wherein the microorganisms of the composition comprise a fecal microbiota of a non-human mammal.

16. The composition of claim 1, wherein the microorganisms of the composition comprise substantially a fecal microbiota of a non-human mammal.

17. (canceled)

18. (canceled)

19. (canceled)

20. The composition of claim 1, wherein the composition comprises one or more microbial taxa and/or strains obtained by fermentation.

21. The composition of claim 20, wherein the microorganisms obtained by fermentation comprise microorganisms of: at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, and/or Megamonas.

22. (canceled)

23. The composition of claim 1, wherein the microorganisms of the composition are a combination of a fecal microbiota of a non-human mammal or substantially a fecal microbiota of a non-human mammal, and one or more microbial taxa, the microbial taxa being cultured, isolated, enhanced, obtained from fermentation, or a combination thereof.

24. (canceled)

25. (canceled)

26. (canceled)

27. (canceled)

28. The composition of claim 1, wherein the composition comprises a capsule comprising the microorganisms, the microorganisms being frozen, freeze-dried, lyophilized, spray-dried, or a combination thereof.

29. The composition of claim 28, wherein one or more excipients are present and at least one of the one or more excipients is selected from the group consisting of glycol, glycerol, vegetable glycerol, dimethylsulfoxide, 1,3-propanediol, ethanol, polyvinyl alcohol, propylene glycol, trimethylene glycol, diethylene glycol, polyethylene glycol, polypropylene glycol, polyethylene oxide, mannitol, D-Mannitol, D-Sorbitol, sorbitol, dulcitol, xylitol, glucose, D-Glucose, xylose, sucrose, lactose, maltose, trehalose, raffinose, dextran, mannan, dextrin, gum arabic (acacia), acetamide, methylacetamide, dimethylformamide, dimethyl-acetamide, succinimide, methylpyrrolidone, polyvinylpyrrolidone, proline, glycine, glutamic acid, aminobutyric acid, glutaric acid, ammonium acetate, cysteines, EDTA, blood serum, fetal calf serum, albumins, bovine serum albumin (BSA), gelatin, casein hydrolysate, starch hydolysate, hydroxypropyl cellulose, methylcellulose, ethylcellulose, hydroxypropyl methylcellulose, peptones, shell extract, glycoproteins, mucin, valinomycin, gramicidin, yeast extract, malt extract, skim milk, dairy milk, soy milk, fish oil, honey, polysorbate 80, polysorbate 20, polysorbate 60, polyethylene glycol p-(1,1,3,3-tetramethylbutyl)-phenyl ether, macrocyclon, glycine betaine, and combinations thereof.

30. A method of preparing an oral composition for treating a disease or condition in a non-human animal, the method comprising:
(a) Obtaining samples of fecal material from one or more individual non-human mammal donors, the donors being of the same species;
(b) Screening the fecal material samples for at least one pathogen;
(c) Eliminating the fecal material sample(s) with pathogens, if any;
(d) Screening the fecal material samples remaining after pathogen screening for bacterial diversity;
(e) Eliminating the fecal material sample(s) that do not meet the following criteria, if any:
The absence of specific pathogens; acceptable fecal consistency; the presence of microorganisms of at least one taxon in group (a) and/or at least one taxon in group (b):
Group (a): the following taxa: at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, Megamonas;
Group (b): the following taxa: Dorea, at least one member of the order Clostridiales, Sutterella, Eubacterium, Collinsella, at least one member of the family Erysipelotrichaceae, Megamonas, at least one member of the family Ruminococcaceae, Clostridium, at least another member of the family Clostridiaceae, at least another member of the family Lachnospiraceae that is not Blautia and that is not Dorea, Faecalibacterium,
(f) If criteria are met, processing at least a portion of at least one of the fecal material sample(s) to form a composition for oral administration to a non-human mammal.

31. The method of claim 30, wherein donors are screened for health, and only healthy donors are included in step (a).

32. The method of claim 30, wherein if all fecal samples are eliminated after step (e), repeating steps (a)-(e) on one or more occasions until at least one fecal material sample is not eliminated after step (e).

33. The method of claim 30, wherein the non-human mammal donors are cats; and
wherein the pathogens screened for comprise Clostridium difficile toxins A and B, Cryptosporidium spp, Salmonella spp, feline coronavirus, feline parvovirus (Panleukopenia), Giardia spp, canine parvovirus 2, and Tritrichomonas foetus.

34. The method of claim 33, wherein in step (e) microorganisms of at least one of the taxa of group (a) are present.

35. The method of claim 34, wherein the microorganisms of the at least one of the taxa of group (a) are present at an abundance at least equal to the median abundance of healthy cats.

36. The method of claim 30, wherein the non-human mammal donors are dogs; and
wherein the pathogens screened for include Clostridium difficile toxins A and B, Cryptosporidium spp, Salmonella spp, Giardia spp, canine parvovirus 2, Clostridium perfringens antigen, alpha toxin, and beta toxin.

37. The method of claim 36, wherein in step (e) microorganisms of at least one of the taxa of group (b) are present.

38. The method of claim 37, wherein the microorganisms of the at least one of the taxa of group (b) are present at an abundance at least equal to the median abundance of healthy dogs.

39. (canceled)

40. (canceled)

41. (canceled)

42. (canceled)

43. The method of claim 30, wherein processing at least a portion of at least one of the fecal samples comprises removing any outside contamination, optionally adding one or more excipients to the fecal sample, and subsequently freezing, freeze-drying, spray-drying, lyophilizing, or a combination thereof, the fecal sample and optional excipient(s) to form a solid.

44. The method of claim 43, wherein the processing further comprises reducing the size of at least a portion of the solid to form a powder, and subsequently at least partially filling one or more capsules with at least a portion of the powder.

45. A method of treating a non-human mammal suffering from at least one disease or condition, the method comprising:
administering to anon-human mammal a composition comprising microorganisms, the microorganisms comprising:
(a) microorganisms of at least one of the following taxa: Ruminoccoccus, at least one member of the family Clostridiaceae, at least one member of the family Lachnospiraceae that is not Blautia and that is not Dorea, Blautia, Fusobacterium, Dorea, at least one member of the order Clostridiales, Bacteroides, Sutterella, Eubacterium, Collinsella, Megamonas, at least one member of the family Ruminococcaceae, Clostridium, Prevotella, at least another member of the family Clostridiaceae, at least another member of the family Lachnospiraceae that is not Blautia and that is not Dorea, Faecalibacterium; and
(b) microorganisms of at least one of the following taxa, which may be the same as or different from the microorganisms of the at least one taxa in (a): at least one member of the order Clostridiales, at least one member of the family Ruminococcaceae, Clostridium, at least one member of the family Clostridiaceae, Sutterella, Eubacterium, Oscillospira, Ruminococcus, Coprococcus, Parabacteroides, Fusobacterium, Faecalibacterium, Megamonas;
and/or
microorganisms of at least one of the following taxa, which may be the same as or different from the microorganisms of the at least one taxa in (a): Dorea, at least one member of the order Clostridiales, Sutterella, Eubacterium, Collinsella, at least one member of the family Erysipelotrichaceae, Megamonas, at least one member of the family Ruminococcaceae, Clostridium, at least another member of the family Clostridiaceae, at least another member of the family Lachnospiraceae that is not Blautia and that is not Dorea, Faecalibacterium.

46. The method of claim 45, where the composition is a solid composition for oral administration.

47. (canceled)

48. The method of claim 47, wherein at least one disease or condition is a gastrointestinal disease or condition.

49. The method of claim 48, wherein the at least one gastrointestinal disease or condition comprises colitis, constipation, acute or chronic diarrhea, gastritis, gastroenteritis, inflammatory bowel disease, irritable bowel syndrome, pancreatitis, small intestinal malabsorption, vomiting, regurgitation, hemorrhagic gastroenteritis, and/or inflammatory bowel disease.

50. The method of claim 47, wherein the disease or condition comprises atopic dermatitis, dermatitis, one or more skin conditions, diabetes, kidney disease, or a combination thereof.

51. The method of claim 47, wherein at least one disease or condition is infection with Tritrichomonas foetus, Campylobacter, Clostridium difficile, Clostridium perfringens, Parvovirus, or a combination thereof.

52. The method of claim 47, wherein at least one disease or condition is a food allergy, food sensitivity, and/or a reaction to a food.

53. The method of claim 47, wherein the solid oral composition is a capsule and/or a tablet, and a unit dosage is one capsule or one tablet, and administration comprises administration of one, two, or three unit dosages one, two, or three times daily for a period of 4 days to 30 days.

54. (canceled)

55. The method of claim 53, wherein the administration of the unit dosage(s) is concurrent with the consumption of food.

56. The method of claim 55, wherein fiber is added to the food, and/or the food is a high fiber food.

57. (canceled)

58. The method of claim 53, wherein the administration comprises concurrent administration of fiber in the amount of 0.01 to 10 mg/kg.

59. The method of claim 58, wherein concurrent administration of fiber comprises administration of one or more capsules comprising fiber, one or more tablets comprising fiber, or both one or more capsules comprising fiber and one or more tablets comprising fiber.

60. The method of claim 45, wherein the non-human mammal is a cat.

61. The method of claim 45, wherein the non-human mammal is a dog.

62. (canceled)

63. (canceled)

64. The method of claim 45, wherein the non-human mammal is a cat and the administration results in a significant change in at least one of the following microorganisms of the microbiota of the non-human mammal: Blautia, Oscillospira, Ruminococcus, Lachnospiraceae g1, Clostridiales f1.

65. The method of claim 45, wherein the non-human mammal is a dog and the administration results in at least one of the following microorganisms of the microbiota of the patient: Bacteroides, Enterococcus, Streptococcus, Ruminococcus, Blautia, Dorea, Prevotella, Clostridiaceae g1, Lachnospiraceae g1, Coprococcus, Oscillospira, Eubacterium, Clostridiales f1.
